(19) United States
(12) Patent Application Publication
(10) Pub. No.: US 2026/0009787 A1
(43) Pub. Date: Jan. 8, 2026

(54) MMP-14 POTENCY ASSAY FOR MESENCHYMAL STEM CELLS

(71) Applicant: LONGEVERON INC., Miami, FL (US)

(72) Inventors: Joshua M. HARE, Miami Beach, FL (US); Brian G. RASH, Miami, FL (US)

(73) Assignee: LONGEVERON INC., Miami, FL (US)

(21) Appl. No.: 19/328,972

(22) Filed: Sep. 15, 2025

Related U.S. Application Data
(63) Continuation-in-part of application No. PCT/US2025/020930, filed on Mar. 21, 2025.
(60) Provisional application No. 63/568,286, filed on Mar. 21, 2024.
(60) Provisional application No. 63/702,937, filed on Oct. 3, 2024.
(60) Provisional application No. 63/703,501, filed on Oct. 4, 2024.

(51) Int. Cl. G01N 33/50

(52) U.S. Cl. CPC G01N 33/5073; G01N 33/5023

(57) ABSTRACT

Compositions and methods are disclosed herein for the treatment of Alzheimer's disease and hypoplastic left heart syndrome (HLHS) with allogeneic mesenchymal stem cells (MSCs). The methods of treatment involve an administration of a composition of allogeneic mesenchymal stem cells to a subject in need thereof, wherein the effectiveness of the treatment methods can be determined through the measurement of specific biomarkers.

### CROSS-REFERENCE TO RELATED APPLICATIONS

This application is a continuation-in-part of PCT Application No. PCT/US2025/020930, filed on Mar. 21, 2025, entitled “MMP-14 POTENCY ASSAY FOR MESENCHYMAL STEM CELLS”, which claims priority to U.S. Provisional Patent Application No. 63/568,286, filed on Mar. 21, 2024, entitled “MMP-14 POTENCY ASSAY FOR MESENCHYMAL STEM CELLS”, U.S. Provisional Patent Application No. 63/702,937, filed on Oct. 3, 2024, entitled “MMP-14 Potency Assay”, and U.S. Provisional Patent Application No. 63/703,501, filed on Oct. 4, 2024, and entitled “MMP-14 POTENCY ASSAY FOR MESENCHYMAL STEM CELLS”. The entirety of each of PCT Application No. PCT/US2025/020930, U.S. Provisional Patent Application No. 63/568,286, U.S. Provisional Patent Application No. 63/702,937, and U.S. Provisional Patent Application No. 63/703,501 is hereby incorporated by reference.

### FIELD OF THE INVENTION

The present application relates to methods and compositions for the treatment of Alzheimer's disease and/or hypoplastic left heart syndrome (HLHS) in subjects in need thereof. Some embodiments are drawn to compositions comprising a therapeutically effective amount of allogeneic mesenchymal stem cells (MSCs), which are used to alleviate the symptoms of Alzheimer's disease (AD), such as without limitation increased systemic inflammation, or alleviate symptoms of hypoplastic left heart syndrome (HLHS). Other embodiments are drawn to methods of treatment wherein subjects suffering from symptoms of Alzheimer's disease (AD) or hypoplastic left heart syndrome (HLHS) are administered compositions including a therapeutically effective amount of MSCs. The effectiveness of these treatments (e.g., for AD or HLHS) can be evaluated through measuring the concentrations of specific biomarkers in subjects after administration of compositions comprising MSCs. The effectiveness of these treatments (e.g., for AD) can also be evaluated by examining changes in their brain activity or morphology, and/or determining if their cognitive functioning has improved after treatment. Some embodiments for measuring the effectiveness of these treatments pertain to the ability of the MSCs to inhibit matrix metalloproteinase-14 (MMP-14). Other embodiments of the invention pertain to methods for assessing the potency of MSCs and their ability to inhibit MMP-14.

### BACKGROUND

Alzheimer's disease (AD) involves complex pathology and encompass diverse mechanisms in addition to β-amyloid deposition and neurofibrillary tangles. There is growing recognition that a pro-inflammatory state contributes to the ensuing dementia. In this regard, proinflammatory cytokines are abundant in the vicinity of amyloid deposits and neurofibrillary tangles, and an association exists between systemic inflammation and β-amyloid accumulation. AD is further characterized by impaired neurovasculature that contributes to adverse outcomes. Resulting compromise of the blood-brain barrier (BBB) can impair exchange across the endothelium, leading to inefficient clearance and accumulation of amyloid-β peptide (AβP) in the brain.

Due to the complex nature of AD progression, the use of biomarkers to predict AD onset and progression remains challenging. Although the concentration of β-amyloid deposits and neurofibrillary tangles can be used to diagnose or predict the onset of AD, there are individuals that have been shown to possess a significant quantity of amyloid deposits and neurofibrillary tangles at autopsy, which would qualify them for a diagnosis of AD despite the records of these individuals never showing a history of dementia.

Hypoplastic left heart syndrome (HLHS) is a rare cardiac birth defect in which the components of the left ventricle (LV) are variably underdeveloped to the extent that the LV is unable to support systemic circulation (Ohye, R. G. et al., “Comparison of shunt types in the Norwood procedure for single-ventricle lesions”, New England Journal of Medicine, 2010; 362 (21): 1980-1992). The only reason HLHS patients are alive is due to the presence of patent ductus arteriosus (PDA) between the pulmonary artery (PA) and aorta in neonates, which allows the right ventricle (RV) to support systemic circulation. However, the duct naturally closes in the first few days after birth, and in the absence of this duct-dependent systemic circulation, HLHS babies do not survive without early surgical intervention (Barron et al., “Hypoplastic left heart syndrome”, The Lancet, 2009; 374 (9689): 551-564). In addition to an underdeveloped LV, HLHS manifests with variable anatomical defects, including a hypoplastic aorta and aortic arch, and mitral valve atresia or stenosis. Depending on the degree of these abnormalities, HLHS can present with a spectrum of various severities.

In an HLHS heart, deoxygenated blood returns to the right atrium (RA), similar to blood flow seen in a normal heart. But oxygenated blood coming from pulmonary veins into the left atrium (LA), instead of being ejected in LV, traverses into the RA via a defective atrial septum (a patent foramen ovale) and mixes with deoxygenated blood, creating a cyanotic condition. This mixed blood in the RV then proceeds into the PA and splits into two directions. A fraction of this mixed blood flows into the lungs for oxygenation, similar to blood flow seen in a normal heart. The remaining blood flow proceeds into the aorta through a PDA, which enables systemic circulation. However, without intervention, the duct closes and the right side of the heart is no longer be able to support circulation, revealing the insufficiency of the left heart in supporting systemic circulation which has inescapable fatal consequences (Barron et al., 2009; Ohye et al., 2010).

Currently, diagnosis of HLHS is made prenatally in most cases by simply observing the absence of the normal ‘four-chamber’ heart using echocardiography imaging. Although there have been chromosomal and genetic abnormalities associated with HLHS, the genetic factors are variable and heterogeneous (Rychik, J., “Hypoplastic left heart syndrome: from in-utero diagnosis to school age”. Paper presented at the Seminars in Fetal and Neonatal Medicine (2005)).

Although HLHS babies are born with normal body weight and height, growth challenges become apparent with the manifestations of the syndrome after birth and the significant metabolic stress from the necessary open-heart reconstructive surgeries (Kelleher, Laussen, Teixeira-Pinto, & Duggan. “Growth and correlates of nutritional status among infants with hypoplastic left heart syndrome (HLHS) after stage 1 Norwood procedure”, Nutrition, 2006; 22 (3): 237-244). Somatic growth is measured in terms of age- and gender-adjusted Z-scores which is the standard deviation above or below the mean of the general population. A Z-score of 0 is equivalent of 50th percentile, with positive addition going to higher percentiles and vice versa. Kelleher et al. showed that at the time of hospital admission for Stage II operation-60% of infants with HLHS were below the fifth weight-for-age percentile (weight-for-age Z score of <−1.65), while-40% were below the fifth length-for-age percentile (height-for-age Z score <−1.65). Longer length of hospital stay, longer ICU stay, and frequency of readmissions were independently correlated with poor somatic growth (Kellcher et al., 2006).

As described above, the variably underdeveloped components of LV pose a life-threatening condition in HLHS patients. HLHS is fatal shortly after birth in the absence of surgical intervention, and it accounts for 25% to 40% of all neonatal cardiac mortality (Barron et al., 2009). The inherent cyanotic nature of HLHS, along with the underdeveloped aorta, also lead to coronary insufficiency, which is a major cause of adverse cardiac events. Additionally, the univentricular status of HLHS even after reconstructive surgery causes abnormal loading conditions in the RV, because the RV serves as the sole systemic pumping chamber. This in turn can trigger detrimental remodeling, despite available cardiac management. Potential manifestations are dilatation (enlargement of the cardiac chamber), myocardial hypertrophy (thickening of the heart walls), and fibrosis (death of cardiac cells which are replaced by scar tissue), which can ultimately lead to heart failure (Wehman et al., “Mesenchymal stem cells preserve neonatal right ventricular function in a porcine model of pressure overload”. Am. J. Physiol. Heart. Circ. Physiol., 2016; 310 (11): HI 816-1826). Heart failure can lead to need for heart transplant and/or death.

Management options for HLHS include reconstructive surgery, heart transplantation, and comfort care (also known as compassionate care). These options are time sensitive and parents of HLHS babies undergo a great deal of stress at the time of decision-making (Toebbe, et al., “Hypoplastic left heart syndrome: parent support for early decision making”, J. Pediatr. Nurs., 2013; 28 (4): 383-392).

The 1-year survival for HLHS babies undergoing reconstructive surgery ranges from 20% to 60% (Siffel et al., “Survival of Children With Hypoplastic Left Heart Syndrome”. Pediatrics, 2015; 136 (4): e864-870), and these procedures require several follow-up admissions and additional surgical interventions. Survivors will have limited physical capacity, increased risk of cognitive impairment, and other long-term complications (Kon et al., “How pediatricians counsel parents when no best-choice management exists: lessons to be learned from hypoplastic left heart syndrome”, Arch. Pediatr. Adolesc. Med., 2004; 158 (5): 436-441). In those cases that opt for reconstructive surgeries, if the clinical outcomes are not favorable post-surgery, enlisting for cardiac transplant is the final end of life option.

Regardless, the overall 1-year survival for those undergoing surgery or transplant is-40% (Kon et al., 2004), a significant and devastating mortality rate, which calls for novel therapeutic strategies to improve outcomes. With technical advances in reconstructive surgeries, the survival following each staged procedure has improved over the past decades. However, there is still significant operative mortality, especially with Stage I (Norwood) and the period between Stage I and II (Siffel et al., 2015). Morris et al. reported 26% neonatal mortality (by day 28 of life) in 463 infants with HLHS from a 1999-2007 Texas Birth Defects Registry (Morris et al., “Prenatal diagnosis, birth location, surgical center, and neonatal mortality in infants with hypoplastic left heart syndrome”. Circulation, (2014) 129 (3), 285-292). In-hospital mortality following Norwood surgery was shown to reduce from 40.4% in 1984-1988 era to 15.7% in 2009-2014 (Mascio et al., “Thirty years and 1663 consecutive Norwood procedures: has survival plateaued?” J. Thorac. Cardiovasc. Surg., (2019) 158 (1), 220-229). The one-year survival estimates for HLHS range from 20% up to 74% (Ohye et al., 2010; Siffel et al., 2015). A 2018 study showed that regardless of prenatal vs postnatal diagnosis of HLHS, the 1-year survival is approximately 60% (Alabdulgader, “Survival analysis: prenatal vs. postnatal diagnosis of HLHS”. J. Invasive Noninvasive Cardiol., 2018; 1:8-12). Consistently, Son et al. also demonstrated freedom from death or transplant to be just under 60% at 1-year post-Norwood operation (Son et al., “Prognostic value of serial echocardiography in hypoplastic left heart syndrome”. Circulation: Cardiovascular Imaging, 2018; 11 (7): e006983). In the SVR trial, 6-year transplant-free survival was reported as 60%. So, while we have seen improvements in outcomes, the mortality rate for HLHS patients remains dismal.

Taken together, neonates, infant and children shoulder the heavy burden of morbidity and mortality from HLHS. Even with the most advanced standard of care options, there is significant mortality in the young ages that reaches 60% by 15 years of age (Mahle et al., “Survival after reconstructive surgery for hypoplastic left heart syndrome: a 15-year experience from a single institution”, Circulation, 2000; 102 (19): III-141).

### SUMMARY OF THE INVENTION

Addressing neuropathological features of AD simultaneously could offer therapeutic advantages and lead to novel treatment strategies. Medicinal signaling cells (MSCs, also known as mesenchymal stem cells) are multipotent cells when administered in vitro, with pleiotropic mechanisms of action (MOAs), including without limitation anti-inflammatory properties, ability to improve vascular function, and/or promotion of intrinsic tissue repair and regeneration, among others. MSCs may traffic to sites of inflammation and damage and thus could target sites of neuroinflammation in AD. MSCs can also secrete numerous bioactive molecules that stimulate endogenous stem cell recruitment, proliferation, and differentiation, inhibit apoptosis and fibrosis, and stimulate neovascularization.

MSCs can also regulate host stem cell niches through paracrine activity and heterocellular coupling to promote intrinsic repair and regeneration. Finally, MSCs are immunoevasive and/or immunoprivileged, permitting allogeneic use, and have an acceptable safety profile in clinical trials. These immunoprivileged and/or immunoevasive properties allow MSCs to have the potential to be an “off-the-shelf” therapy that is readily available and accessible to broad patient populations due to their undetectable levels of major histocompatibility complex class II (MHC-II) molecules and low levels of major histocompatibility complex class I (MHC-I) molecules.

There are some preclinical data supporting efficacy of MSCs in AD. In animal models, MSCs may cross the BBB, promote neurogenesis, inhibit β-amyloid deposition and promote clearance thereof, reduce apoptosis, promote hippocampal neurogenesis, improve dendritic morphology, and/or improve behavioral and spatial memory performance. These beneficial effects may be associated with decreased inflammation, increased Aβ-degrading factors and Aβ clearance, decreased hyperphosphorylated tau, and/or elevated alternatively activated microglial markers. These benefits may appear, at least in part, due to Aβ-induced MSC release of chemoattractants, which may recruit alternative microglia into the brain to reduce Aβ deposition. MSCs have been reported to be effective in young AD-model mice prior to Aβ accumulations, leading to significant decreases in cerebral Aβ deposition and a significant increase in expression of pre-synaptic proteins. Impressively, these effects were sustained for at least 2 months, which suggests that MSCs could potentially be effective as an interventional therapeutic in prodromal AD.

Accordingly, the application seeks to provide methods of treatment for AD, wherein the methods include the use of compositions containing MSCs. In addition, the application also seeks to provide methods that can accurately measure the potential safety of MSCs and evaluate their efficacy in the treatment of AD and/or in the alleviation of AD symptoms in subjects in need thereof. An objective of the present application is to provide methods of treatment of AD, alleviation of symptoms of AD, and/or inhibiting AD disease progression (simply referred to as “treatment of AD” hereinafter). The method of treatment of AD includes administering a composition, wherein the composition includes a therapeutic amount of allogeneic MSCs, to a subject in need thereof.

Further, novel therapeutic options to increase transplant-free survival and quality of life are desperately needed to improve the current outlook and long-term outcomes of HLHS. Therefore, the application further seeks to provide methods of treatment for HLHS, wherein the methods include the use of compositions containing MSCs. In addition, the application also seeks to provide methods that can accurately measure the potential safety of MSCs and evaluate their efficacy in the treatment of HLHS in subjects in need thereof. Accordingly, another objective of the present application is to provide methods of treatment of HLHS, in a subject in need thereof. The method of treatment of HLHS includes administering a composition, wherein the composition includes a therapeutic amount of allogeneic MSCs, to a subject in need thereof.

In some embodiments, the allogenic MSCs may include a Longeveron formulation of allogenic human MSCs, which may also be referred to as LOMECEL-B™ cells. Further uses and preparation of useful stem cells, including without limitation LOMECEL-B™ brand mesenchymal cells, may be found in the following United States Patent Application Publications, the entirety of each of which is incorporated by reference herein: US20190038742A1; US20190290698A1; and US20200129558A1. LOMECEL-B™ cell formulations are also known as “Laromestrocel.”

In some embodiments, the composition for treatment of AD or HLHS is configured to perform one or more therapeutic functions by preventing tyrosine kinase with immunoglobulin and epidermal growth factor homology domains (TIE2) from degradation.

Additionally, and/or alternatively, in some embodiments, the composition for treatment of AD or HLHS may include allogeneic human microglial cells (HMCs).

In some embodiments, the composition for treatment of AD or HLHS may include a dosage of approximately 20×10⁶ MSCs. In some embodiments, the composition may include a dosage of approximately 100×10⁶ MSCs. In some embodiments, the composition may include a dosage of any amount between 20×10⁴ and 100×10⁸ MSCs. As nonlimiting examples, the composition may include a dosage of approximately 25×10⁴, approximately 30×10⁴, approximately 35×10⁴, approximately 40×10⁴, approximately 45×10⁴, approximately 50×10⁴, approximately 55×10⁴, approximately 60×10⁴, approximately 65×10⁴, approximately 70×10⁴, approximately 75×10⁴, approximately 80×10⁴, approximately 85×10⁴, approximately 90×10⁴, approximately 95×10⁴, approximately 5×10⁶, approximately 10×10⁶, approximately 15×10⁶, approximately 20×10⁶, approximately 25×10⁶, approximately 30×10⁶, approximately 35×10⁶, approximately 40×10⁶, approximately 45×10⁶, approximately 50×10⁶, approximately 55×10⁶, approximately 60×10⁶, approximately 65×10⁶, approximately 70×10⁶, approximately 75×10⁶, approximately 80×10⁶, approximately 85×10⁶, approximately 90×10⁶, approximately 95×10⁶, approximately 5×10⁸, approximately 10×10⁸, approximately 15×10⁸, approximately 20×10⁸, approximately 25×10⁸, approximately 50×10⁸, approximately 100×10⁸, and/or the like. As further nonlimiting examples, the composition may include a dosage of between 20×10⁴ and 20×10⁵ MSCs, between 20×10⁵ and 20×10⁶ MSCs, between 20×10⁶ and 25×10⁶ MSCs, between 25×10⁶ and 30×10⁶ MSCs, between 30×10⁶ and 35×10⁶ MSCs, between 35×10⁶ and 40×10⁶ MSCs, between 40×10⁶ and 45×10⁶ MSCs, between 45×10⁶ and 50×10⁶ MSCs, between 50×10⁶ and 55×10⁶ MSCs, between 55×10⁶ and 60×10⁶ MSCs, between 60×10⁶ and 65×10⁶ MSCs, between 65×10⁶ and 70×10⁶ MSCs, between 70×10⁶ and 75×10⁶ MSCs, between 75×10⁶ and 80×10⁶ MSCs, between 80×10⁶ and 85×10⁶ MSCs, between 85×10⁶ and 90×10⁶ MSCs, between 90×10⁶ and 95×10⁶ MSCs, between 95×10⁶ and 100×10⁶ MSCs, between 100×10⁶ and 100×10⁷ MSCs, or between 100×10⁷ and 100×10⁸ MSCs. Additionally, and/or alternatively, in some embodiments, the composition for treatment of AD or HLHS may include allogeneic human microglial cells (HMCs).

In some embodiments, the method for treatment of AD, as described above, may further include examining a cerebral spinal fluid of the subject before and after administration of the composition including the therapeutically effective amount of the allogenic MSCs.

In some embodiments, the method for treatment of AD, as described above, may further include determining if a change in the cortical amygdaloid transition area of the subject has occurred after administration of the composition including the therapeutically effective amount of the allogenic MSCs.

In some embodiments, the method for treatment of AD, as described above, may further include measuring a cognitive or quality-of-life function of the subject suffering from symptoms of AD before and after administration of the composition including the therapeutically effective amount of the allogenic MSCs. As nonlimiting examples, the cognitive or quality-of-life function may be measured using Composite Alzheimer's Disease Score (CADS), Montreal Cognitive Assessment (MoCA), Alzheimer's Disease cooperative study-Activities of Daily Living Scale (ADCS-ADL), Mini-Mental State Examination (MMSE), and/or the like.

In some embodiments, the method for treatment of AD or HLHS, as described above, may further include evaluating one or more biomarkers in the subject suffering from symptoms of AD or HLHS before and after administration of the composition including the therapeutically effective amount of the allogenic MSCs.

Another objective of the present application is to provide novel biomarkers for diagnosing and evaluating the progression of AD or HLHS and the effectiveness of the treatment methods associated thereto.

In some embodiments, a biomarker may include one or more MRI biomarkers. In some cases, the one or more MRI biomarkers may be evaluated using a volume measurement, such as without limitation a volume measurement of a whole brain, or one or more subregions of a whole brain, including without limitation a lateral ventricle, grey matter, a medial temporal cortex, a hippocampus, a thalamus, white matter, and/or a cingulate cortex, among others. In some cases, the volume measurement may indicate a suppressed decrease in volume. In some other cases, the volume measurement may indicate a suppressed increase in volume.

In some embodiments, evaluating the one or more MRI biomarkers may include evaluating the one or more MRI biomarkers using diffusion tensor imaging (DTI), arterial spin labeling (ASL), and/or other techniques similar thereto.

In some embodiments, the one or more biomarkers for diagnosing and evaluating the progression of AD may include a change in whole brain volume. In preferred embodiments, the decrease in whole brain volume is reduced in the subject in need thereof suffering from AD symptoms after administration of allogeneic MSCs to said subject. As nonlimiting examples, the reduction in whole brain volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in volume for one or more subregions of a whole brain, including but not limited to: a lateral ventricle, grey matter, a medial temporal cortex, a hippocampus, a thalamus, white matter, and a cingulate cortex, consistent with details described elsewhere in this disclosure. In preferred embodiments, the change in volume is reduced in the subject in need thereof suffering from AD symptoms after administration of allogeneic MSCs to said subject. As nonlimiting examples, the reduction in change in volume can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%, compared with placebo.

In some embodiments, the one or more biomarkers for diagnosing and evaluating the progression of AD may include a change in lateral ventricle volume. In preferred embodiments, the increase in lateral ventricle volume may be reduced in the subject in need thereof suffering from AD symptoms after administration of allogeneic MSCs to said subject. As nonlimiting examples, the reduction in lateral ventricle volume increase can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in grey matter volume. In preferred embodiments, the decrease in grey matter volume may be reduced in the subject in need thereof suffering from AD symptoms after administration of allogeneic MSCs to said subject. As nonlimiting examples, the reduction in grey matter volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in medial temporal cortex volume. In preferred embodiments, the decrease in medial temporal cortex volume may be reduced in the subject in need thereof suffering from AD symptoms after administration of allogeneic MSCs to said subject. As nonlimiting examples, the reduction in medial temporal cortex volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in hippocampus volume. In preferred embodiments, the decrease in hippocampus volume may be reduced in the subject in need thereof suffering from AD symptoms after administration of allogeneic MSCs to said subject. As nonlimiting examples, the reduction in hippocampus volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in thalamus volume. In preferred embodiments, the decrease in thalamus volume may be reduced in the subject in need thereof suffering from AD symptoms after administration of allogeneic MSCs to said subject. As nonlimiting examples, the reduction in thalamus volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in white matter volume. In preferred embodiments, the decrease in white matter volume may be reduced in the subject in need thereof suffering from AD symptoms after administration of allogeneic MSCs to said subject. As nonlimiting examples, the reduction in white matter volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the one or more biomarkers for diagnosing and evaluating the progression of AD may include a change in cingulate cortex volume. In preferred embodiments, the decrease in cingulate cortex volume may be reduced in the subject in need thereof suffering from AD symptoms after administration of allogeneic MSCs to said subject. As nonlimiting examples, the reduction in cingulate cortex volume decrease can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, evaluating the one or more biomarkers for AD or HLHS may include measuring a concentration of one or more whole-blood biomarkers, one or more blood-plasma biomarkers, one or more blood-serum biomarkers, and/or the like.

In some embodiments, evaluating the one or more biomarkers may include measuring a level or concentration of MMP-14, TIE2 including soluble TIE2 (STIE2), an angiopoietin-1 receptor, cotaxin 1, cotaxin 2, cotaxin 3, tissue-inhibitor-of-metalloprotease-2 (TIMP2), active glucose-dependent insulinotropic polypeptide (GIP), intact GIP, placental growth factor (plGF), amyloid beta peptide with 40 amino acids (Aβ40), interleukin 1β (IL-1β), interleukin 2 (IL-2), interleukin 4 (IL-4), interleukin 6 (IL-6), interleukin 10 (IL-10), and/or interleukin 13 (IL-13), among others.

Another objective of the present application is to provide methods for evaluating potency of allogeneic MSCs using a potency assay. The potency assay can include an MMP-14 inhibition assay, any form of molecular assay for TIMP1, TIMP2, or TIMP3, or VEGF-A, including enzyme-linked immunosorbent assay (ELISA), an electro-chemiluminescence assay such as a meso scale discovery (MSD) assay, an assay based on mass spectrometry, or any other assay method deemed suitable for measuring the concentration of one or more of the foregoing analytes by a person of ordinary skill in the art, upon reviewing the entirety of the disclosure.

In some embodiments, the potency assay may include an MMP-14 inhibition assay. Accordingly, in some embodiments, one or more biomarkers for determining the potency of MSCs to treat AD in a subject in need thereof may include MMP-14. In preferred embodiments, the inhibition of MMP-14 may be directly related to the ability of MSCs to treat AD. The inhibition of MMP-14 may be at any inhibition level deemed relevant and/or reasonable by a person of ordinary skill in the art, upon reviewing the entirety of this disclosure. As nonlimiting examples, the inhibition of MMP-14 can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, the potency assay may include a TIMP2 enzyme-linked immunosorbent assay (ELISA). Accordingly, in some embodiments, one or more biomarkers for determining the potency of MSCs to treat AD in a subject in need thereof may include TIMP2. In preferred embodiments, the expression level of TIMP2 may be directly related to the ability of MSCs to treat AD. The expression level of TIMP2 may be any expression level deemed relevant and/or reasonable by a person of ordinary skill in the art, upon reviewing the entirety of this disclosure. As nonlimiting examples, the expression level of TIMP2 can be from 0 to 1×10⁻⁷ ng/cell/L, from 1×10⁻⁷ ng/cell/L to 2×10⁻⁷ ng/cell/L, from 2×10⁻⁷ ng/cell/L to 3×10⁻⁷ ng/cell/L, from 3×10⁻⁷ ng/cell/L to 4×10⁻⁷ ng/cell/L, from 4×10⁻⁷ ng/cell/L to 5×10⁻⁷ ng/cell/L, from 5×10⁻⁷ ng/cell/L to 6×10⁻⁷ ng/cell/L, from 6×10⁻⁷ ng/cell/L to 7×10⁻⁷ ng/cell/L, from 7×10⁻⁷ ng/cell/L to 8×10⁻⁷ ng/cell/L, from 8×10⁻⁷ ng/cell/L to 9×10⁻⁷ ng/cell/L, from 9×10⁻⁷ ng/cell/L to 1×10⁻⁶ ng/cell/L, and/or the like.

In some embodiments, the potency assay may include a vascular endothelial growth factor A (VEGF-A) assay or a meso scale discovery (MSD) assay. Accordingly, in some embodiments, one or more biomarkers for determining the potency of MSCs to treat AD or HLHS in a subject in need thereof may include VEGF-A. In preferred embodiments, the expression level of VEGF-A may be directly related to the ability of MSCs to treat AD or HLHS. The expression level of VEGF-A may be any expression level deemed relevant and/or reasonable by a person of ordinary skill in the art, upon reviewing the entirety of this disclosure. As nonlimiting examples, the expression level of VEGF-A can be from 0 to 1×10⁻⁵ ng/cell/L, from 1×10⁻⁵ ng/cell/L to 2×10⁻⁵ ng/cell/L, from 2×10⁻⁵ ng/cell/L to 3×10⁻⁵ ng/cell/L, from 3×10⁵ ng/cell/L to 4×10⁻⁵ ng/cell/L, from 4×10⁻⁵ ng/cell/L to 5×10⁻⁵ ng/cell/L, from 5×10⁻⁵ ng/cell/L to 6×10⁻⁵ ng/cell/L, from 6×10⁻⁵ ng/cell/L to 7×10⁻⁵ ng/cell/L, from 7×10⁻⁵ ng/cell/L to 8×10⁻⁵ ng/cell/L, from 8×10⁻⁵ ng/cell/L to 9×10⁻⁵ ng/cell/L, from 9×10⁻⁵ ng/cell/L to 1×10⁴ ng/cell/L, and/or the like.

In some embodiments, the potency assay may include a matrix potency assay. In some embodiments, the potency assay may include or otherwise implement two or more members selected from a group consisting of an MMP-14 inhibition assay, a molecular assay for TIMP1, TIMP2, TIMP3, or VEGF-A, an enzyme-linked immunosorbent assay (ELISA), an electro-chemiluminescence assay, a meso scale discovery (MSD) assay, and an assay based on mass spectrometry. In some embodiments, the method for evaluating potency of allogeneic MSCs may further include measuring a concentration or expression level of IL-6, interleukin 8 (IL-8), and/or the like.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings unless it is so claimed.

FIG. 1 depicts mechanisms of actions (MOAs) of Lomecel-B™ cells for treatment of Alzheimer's disease and/or alleviation of symptoms thereof.

FIG. 2A depicts an exemplary magnetic resonance (MR) image of the cingulate cortex of a subject from Group 1 without treatment (placebo); the image was captured using diffusion tensor imaging (DTI).

FIG. 2B depicts an exemplary MR image of cerebral blood flow (CBF) in the medial temporal cortex of a subject from Group 1 without treatment (placebo); the image was captured using arterial spin labeling (ASL).

FIG. 2C depicts an exemplary MR image of the cingulate cortex of a subject from Group 4 treated with mesenchymal stem cells (MSCs, 4 doses×100 million cells per dose); the image was captured using diffusion tensor imaging (DTI).

FIG. 2D depicts an exemplary MR image of cerebral blood flow (CBF) in the medial temporal cortex of a subject from Group 4 treated with MSCs (4 doses×100 million cells per dose); the image was captured using arterial spin labeling (ASL).

FIG. 3A depicts exemplary volumetry data of whole brain (bilateral), measured as a change from baseline, collected from Groups 1-4 over a course of 39 weeks from the first infusion; while Group 1 (placebo) showed a linear decrease in whole-brain shrinkage, all treatment arms from Groups 2-4 showed numerically superior results, reaching statistical significance for the multi-dosing arms (Groups 3 and 4).

FIG. 3B depicts exemplary volumetry data of whole brain (bilateral), measured as a change in whole brain ratio, collected from Groups 1-4 over a course of 39 weeks from the first infusion; while Group 1 (placebo) showed a linear decrease in whole-brain shrinkage, Groups 2-4 showed numerical improvement at Week 26 and statistically significant improvement at Week 39.

FIG. 4A depicts exemplary volumetry data of lateral ventricles (left and right), measured as a change from baseline, collected from Groups 1-4 over a course of 39 weeks from the first infusion; while Group 1 (placebo) showed a linear increase in the volume of lateral ventricles, all treatment arms in Groups 2-4 showed numerically superior results at Week 39.

FIG. 4B depicts exemplary volumetry data of lateral ventricles (left), measured as a change in lateral ventricles (left) ratio, collected from Groups 1-4 over a course of 39 weeks from the first infusion; while Group 1 (placebo) showed a linear increase in the volume of lateral ventricles, all treatment arms in Groups 2-4 showed numerically superior results at Week 39.

FIG. 4C depicts exemplary volumetry data of lateral ventricles (right), measured as a change in lateral ventricles (right) ratio, collected from Groups 1-4 over a course of 39 weeks from the first infusion; while Group 1 (placebo) showed a linear increase in the volume of lateral ventricles, all treatment arms in Groups 2-4 showed numerically superior results at Week 39.

FIG. 5A depicts exemplary volumetry data of grey matter (left and right), measured as a change from baseline, collected from Groups 1-4 over a course of 39 weeks from the first infusion; while Group 1 (placebo) showed a fairly linear decrease, consistent with a loss of neurons and synaptic connections, all treatment arms in Groups 2-4 showed numerically superior results at Week 39.

FIG. 5B depicts exemplary volumetry data of grey matter (left), measured as a change in grey matter (left) ratio, collected from Groups 1-4 over a course of 39 weeks from the first infusion; while Group 1 (placebo) showed a fairly linear decrease, consistent with a loss of neurons and synaptic connections, all treatment arms in Groups 2-4 showed numerically superior results at Week 39; in particular, a statistically significant improvement was achieved in pooled Lomecel-B™ group at Week 39.

FIG. 5C depicts exemplary volumetry data of grey matter (right), measured as a change in grey matter (right) ratio, collected from Groups 1-4 over a course of 39 weeks from the first infusion; while Group 1 (placebo) showed a fairly linear decrease, consistent with a loss of neurons and synaptic connections, all treatment arms in Groups 2-4 showed numerically superior results at Week 39.

FIG. 6A depicts exemplary volumetry data of medial temporal cortex (left and right), measured as a change from baseline, collected from Groups 1-4 over a course of 39 weeks from the first infusion; while Group 1 (placebo) showed a linear decrease, consistent with a loss of major memory-forming structures (e.g., hippocampus and amygdala), all treatment arms in Groups 2-4 showed numerically superior results at Weeks 26 and 39; multi-dosing arms reached statistical significance.

FIG. 6B depicts exemplary volumetry data of medial temporal cortex (left), measured as the least squares (LS) mean change in medial temporal cortex ratio, collected from Groups 1-4 over a course of 39 weeks from the first infusion; while Group 1 (placebo) showed a linear decrease, consistent with a loss of major memory-forming structures (e.g., hippocampus and amygdala), all treatment arms in Groups 2-4 showed numerically superior results at Weeks 26 and 39; multi-dosing arms reached statistical significance.

FIG. 6C depicts exemplary volumetry data of medial temporal cortex (right), measured as least squares (LS) mean change in medial temporal cortex ratio, collected from Groups 1-4 over a course of 39 weeks from the first infusion; while Group 1 (placebo) showed a linear decrease, consistent with a loss of major memory-forming structures (e.g., hippocampus and amygdala), all treatment arms in Groups 2-4 showed numerically superior results at Weeks 26 and 39; multi-dosing arms reached statistical significance.

FIG. 7A depicts exemplary volumetry data of hippocampus (left and right), measured as a change from baseline, collected from Groups 1-4 over a course of 39 weeks from the first infusion; while Group 1 (placebo) showed a linear decrease, all treatment arms in Groups 2-4 showed numerically superior results at Week 39.

FIG. 7B depicts exemplary volumetry data of hippocampus (left), measured as the LS mean change in left hippocampus volume, collected from Groups 1-4 over a course of 39 weeks from the first infusion; all treatment arms in Groups 2-4 showed numerically superior results at Week 39; statistically significant improvements for specific Lomecel-B™ treatment arms were achieved at Weeks 26 and 39.

FIG. 7C depicts exemplary volumetry data of hippocampus (right), measured as the LS mean change in right hippocampus volume, collected from Groups 1-4 over a course of 39 weeks from the first infusion; all treatment arms in Groups 2-4 showed numerically superior results at Week 39; statistically significant improvements for specific Lomecel-B™ treatment arms were achieved at Weeks 26 and 39.

FIG. 8A depicts exemplary volumetry data of thalamus (left and right), measured as a change from baseline, collected from Groups 1-4 over a course of 39 weeks from the first infusion; Group 1 (placebo) showed a linear decrease.

FIG. 8B depicts exemplary volumetry data of thalamus (left), measured as the LS mean change in thalamus ratio, collected from Groups 1-4 over a course of 39 weeks from the first infusion; Group 1 (placebo) showed a linear decrease.

FIG. 8C depicts exemplary volumetry data of thalamus (right), measured as the LS mean change in thalamus ratio, collected from Groups 1-4 over a course of 39 weeks from the first infusion; while Group 1 (placebo) showed a linear decrease, a statistically significant improvement was achieved in Lomecel-B™ treatment arms at Week 39.

FIG. 9A depicts exemplary diffusion tensor imaging (DTI) data of white matter (bilateral), measured as a change from baseline in mean diffusivity (MD, left panel) and axial diffusivity (AD, right panel), collected from Groups 1-4 over a course of 39 weeks from the first infusion.

FIG. 9B depicts exemplary DTI data of white matter (bilateral), measured as a change from baseline in fractional anisotropy (FA, left panel) and radial diffusivity (RD, right panel), collected from Groups 1-4 over a course of 39 weeks from the first infusion.

FIG. 9C depicts exemplary DTI data of cingulate cortex (bilateral), measured as a change from baseline in MD (left panel) and AD (right panel), collected from Groups 1˜4 over a course of 39 weeks from the first infusion; while Group 1 (placebo) showed a linear decrease, which is indicative of pathological damage, numerically superior results were achieved in Lomecel-B™ arms for both DTI-MD and DTI-AD.

FIG. 9D depicts exemplary DTI data of cingulate cortex (bilateral), measured as a change from baseline in FA (left panel) and RD (right panel), collected from Groups 1-4 over a course of 39 weeks from the first infusion.

FIG. 9E depicts exemplary DTI data of whole corpus callosum, measured as a change from baseline in MD (left panel) and AD (right panel), collected from Groups 1-4 over a course of 39 weeks from the first infusion.

FIG. 9F depicts exemplary DTI data of whole corpus callosum, measured as a change from baseline in FA (left panel) and RD (right panel), collected from Groups 1-4 over a course of 39 weeks from the first infusion.

FIG. 10A depicts exemplary arterial spin labeling (ASL) data of whole cortex (left panel) and hypometabolic signature (right panel), measured as a change from baseline, collected from Groups 1-4 over a course of 39 weeks from the first infusion.

FIG. 10B depicts exemplary ASL data of frontal cortex (left panel) and parietal cortex (right panel), measured as a change from baseline, collected from Groups 1-4 over a course of 39 weeks from the first infusion.

FIG. 10C depicts exemplary ASL data of temporal cortex (left panel) and occipital cortex (right panel), measured as a change from baseline, collected from Groups 1-4 over a course of 39 weeks from the first infusion.

FIG. 10D depicts exemplary ASL data of cingulate cortex (left) and medial temporal cortex (right), measured as a change from baseline, collected from Groups 1-4 over a course of 39 weeks from the first infusion.

FIG. 10E depicts exemplary ASL data of thalamus (left panel) and striatum (right panel), measured as a change from baseline, collected from Groups 1-4 over a course of 39 weeks from the first infusion.

FIG. 10F depicts exemplary ASL data of whole cortex ASL (left panel) and medial temporal cortex ASL (right panel), measured as a change from baseline, collected from Groups 1-4 over a course of 39 weeks from the first infusion.

FIG. 11A depicts exemplary data describing change in Eotaxin-1 concentration from baseline, collected from Groups 1-4 over a course of 39 weeks from the first infusion.

FIG. 11B depicts exemplary data describing raw mean change in Eotaxin-1 concentration, collected from Groups 1-4 over a course of 39 weeks from the first infusion.

FIG. 11C depicts exemplary data describing change in Eotaxin-2 concentration from baseline, collected from Groups 1-4 over a course of 39 weeks from the first infusion.

FIG. 11D depicts exemplary data describing raw mean change in Eotaxin-2 concentration, collected from Groups 1-4 over a course of 39 weeks from the first infusion.

FIG. 11E depicts exemplary data describing change in Eotaxin-3 concentration from baseline, collected from Groups 1-4 over a course of 39 weeks from the first infusion.

FIGS. 11F-G depict exemplary data describing raw mean change in Eotaxin-3 concentration, collected from Groups 1-4 over a course of 39 weeks from the first infusion.

FIG. 12A depicts exemplary data describing change in active glucose-dependent insulinotropic polypeptide (GIP) concentration from baseline, collected from Groups 1-4 over a course of 39 weeks from the first infusion.

FIG. 12B depicts exemplary data describing raw mean change in active GIP concentration, collected from Groups 1-4 over a course of 39 weeks from the first infusion.

FIG. 12C depicts exemplary data describing change in intact GIP concentration from baseline, collected from Groups 1-4 over a course of 39 weeks from the first infusion.

FIG. 12D depicts exemplary data describing raw mean change in intact GIP concentration, collected from Groups 1-4 over a course of 39 weeks from the first infusion.

FIG. 12E depicts additional exemplary data describing raw mean change in intact GIP concentration, collected from Groups 1-4 over a course of 39 weeks from the first infusion.

FIG. 12F depicts exemplary data describing change in placental growth factor (PIGF) concentration from baseline, collected from Groups 1-4 over a course of 39 weeks from the first infusion.

FIG. 12G depicts exemplary data describing raw mean change in PIGF concentration, collected from Groups 1-4 over a course of 39 weeks from the first infusion.

FIG. 13A depicts exemplary data describing change in TIE2 concentration from baseline, collected from Groups 1-4 over a course of 39 weeks from the first infusion.

FIG. 13B depicts exemplary data describing raw mean change in TIE2 receptor concentration, collected from Groups 1-4 over a course of 39 weeks from the first infusion.

FIG. 13C depicts exemplary data of LS mean change from baseline in TIE2, at 0 (baseline), 4, 8, 12, 16, 26, and 39 weeks from the first infusion, for four patient groups: ● Placebo (Dose ×4); ▴ MSC (25M×1); ▪ MSC (25M×4); ♦ MSC (100M×4); a rise in soluble TIE2 (sTIE2) levels was observed among subjects of Group 1 with mild AD, and such increase is offset by treatment using Lomecel-B™.

FIG. 13D depicts exemplary data of LS mean change from baseline in TIE2, among placebo Group 1 and pooled Lomecel-B™ Groups 2, 3, and 4, at 0 (baseline), 4, 8, 12, 16, 26, and 39 weeks from the first infusion; no difference was observed between above- or below-median matrix metalloproteinase-14 inhibition (MMP-14i) on TIE2 protection; statistical significance was achieved at week 8 for both above- and below-median MMP-14i potency groups.

FIG. 13E depicts exemplary data of LS mean change from baseline in TIE2, among placebo Group 1 and pooled Lomecel-B™ Groups 3 and 4, at 0 (baseline), 4, 8, 12, 16, 26, and 39 weeks from the first infusion; subjects receiving above-median MMP-14i potency Lomecel-B™ showed a more durable TIE2 protection than those who received below-median MMP-14i potency Lomecel-B™.

FIG. 14A depicts exemplary data describing changes from baseline (CFB) in Composite Alzheimer's Disease Score (CADS) of three aggregated groups of subjects, i.e., Group 1 (placebo), Group 3 and 4, and Group 2, 3, and 4, according to MMP-14i potency at 39 weeks from the first infusion.

FIG. 14B depicts exemplary data showing a linear correlation between MMP-14i and TIMP2 levels.

FIG. 15A depicts exemplary data from Montreal Cognitive Assessment (MoCA) responder analysis, outlining MoCA least squares mean changes from baseline to Week 39 among various groups of subjects; subjects who received above-median potency lots of Lomecel-B™ showed superior performance on the MoCA than those who received below-median potency lots of Lomecel-B™.

FIG. 15B depicts exemplary data from Alzheimer's Disease cooperative study-Activities of Daily Living Scale (ADCS-ADL), outlining ADCS-ADL least squares mean changes from baseline to Week 39 among various groups of subjects; subjects who received above-median potency lots of Lomecel-B™ showed superior performance on the ADCS-ADL than those who received below-median potency lots of Lomecel-B™.

FIG. 16 depicts exemplary data from left hippocampal volume responder analysis, outlining left hippocampal volume least squares mean changes from baseline to Week 39 among various groups of subjects; subjects who received above-median potency lots of Lomecel-B™ showed numerically superior performance on the left hippocampal volume responder analysis than those who received below-median potency lots of Lomecel-B™ with no statistical difference; however, the trend was in the same direction as the CADS, MoCA, and ADCS-ADL results described above.

FIG. 17A depicts exemplary data showing a linear correlation between MMP-14i and levels of tissue-inhibitor-of-metalloprotease-2 (TIMP2) among all clinical lots.

FIG. 17B depicts exemplary data showing a linear correlation between levels of VEGF-A and MMP-14i.

FIG. 17C depicts exemplary data showing a linear correlation between levels of VEGF-A and levels of TIMP2.

FIG. 18 depicts an exemplary vector plot of MMP-14i vs TIMP2 vs VEGF-A, with values representing normalized potency; the three potency assay candidates showed a satisfactory correlation across lots of Lomecel-B™.

FIG. 19A depicts exemplary data showing a correlation between levels of fms-related receptor tyrosine kinase 1 (FLT-1) and MMP-14i.

FIG. 19B depicts data showing a correlation between levels of FLT-1 and levels of TIMP2.

FIG. 20A depicts exemplary data showing a correlation between levels of interleukin 6 (IL-6) and levels of VEGF-A.

FIG. 20B depicts an exemplary dataset showing a correlation between levels of IL-6 and levels of TIMP2.

FIG. 20C depicts another exemplary dataset showing a correlation between levels of IL-6 and levels of TIMP2.

FIG. 21A depicts exemplary data showing a correlation between levels of interleukin 8 (IL-8) and levels of VEGF-A.

FIG. 21B depicts an exemplary dataset showing a correlation between levels of interleukin 8 (IL-8) and levels of TIMP2.

FIG. 21C depicts another exemplary dataset showing a correlation between levels of IL-8 and levels of TIMP2.

FIG. 22 depicts exemplary data comparing the MMP-14 inhibition potency between pre-freeze vs post-thaw Lomecel-B™ supernatants across 26 lots of Lomecel-B cells. Lomecel-B™ supernatants showed higher MMP-14 inhibition activity in all 26 tested lots (13 pre-freeze lots and 13 post-thaw lots). Freeze-thaw cycles therefore do not have a significant negative impact on the MMP14 inhibition activity of Lomecel-B™ cells. Instead, the post-thaw supernatant showed an elevated MMP14 inhibition activity compared to the pre-freeze supernatant. These results directly and positively address the impact of cell freeze-thaw cycles on cell potency.

FIG. 23 depicts exemplary data comparing the MMP-14 inhibition potency between pre-freeze vs post-thaw Lomecel-B™ supernatants across 13 lots of Lomecel-B™ cells. MMP-14 inhibition level is normalized to cell density. Lomecel-B™ typically showed higher MMP14 inhibition in post-thaw samples (P4) compared to pre-freeze samples (P2).

FIGS. 24A-B depict exemplary data showing significant inhibition of MMP-14 activity in all three tested Lomecel-B™ lots from the ELPIS Phase I clinical trial. It should be noted that substrate cleavage may not be specific to MMP-14 and could also be mediated by other MMPs in the base media, likely within the fetal bovine serum (FBS).

FIGS. 25A-B depict exemplary data based on two runs of TIMP2 potency assays (ELISA) using pre-freeze and post-thaw Lomecel-B™ supernatants. TIMP2 potency is not affected in the cell supernatants after a freeze thaw cycle (except for lots 014 and 042 in run 1). TIMP2 potency is elevated in most of the post-thaw lots in run 2.

FIGS. 26A-D depict exemplary data comparing TIMP2 potency between pre-freeze and post-thaw Lomecel-B™ supernatants. As seen in FIGS. 26A and 26C, normalized TIMP2 potency is higher in post-thaw P3/P4 harvest supernatant compared to pre-freeze P2 supernatant. In contrast, as seen in FIGS. 26B and 26D, raw TIMP2 concentrations showed no significant difference.

FIGS. 27A-B depict exemplary data comparing TIMP2 concentrations across four lots from the ELPIS Phase I clinical trial. FIG. 27A showed raw TIMP2 concentrations, whereas FIG. 27B showed normalized TIMP2 concentrations instead. All lots showed significant TIMP2 values.

FIG. 28 depicts exemplary data showing normalized TIMP2 concentrations in both P2 (pre-freeze) and P4 (post-thaw) lots across four Lomecel-B lots. P4 values were generally higher than P2 values, consistent with the observations based on an aggregate of all clinical lots.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Alzheimer's disease (AD) is a progressive disease for which there is no current cure. The Food and Drug Administration (FDA) has only recently (2021-2024) approved a small number of antibody therapeutics (Cummings, J., et al., “Anti-Amyloid Monoclonal Antibodies for the Treatment of Alzheimer's Disease”, BioDrugs, 2024; 38 (1): 5-22) designed to treat AD by removing β-amyloid peptide (Aβ or AβP), including Aducanumab, Lecanemab, and Donanemab, which have demonstrated some efficacy at reducing cognitive and functional decline in patients with early AD.

The development and onset of AD is still not fully understood, and this uncertainty has been the cause of many disputes within the field. Indeed, although a majority of researchers in the field of AD development and treatment acknowledge that AβP accumulation plays a part in the progression of the disease, it is still unknown if AβP accumulation is the cause of AD or if it is merely a result of other cellular pathways becoming dysregulated as a result of aging. Additionally, while AβP accumulation and tau-mediated formation of neurofibrillary tangles remain as defining pathological features of AD, non-amyloid and non-tau contributions to AD have been identified that may result in cerebrovascular degradation and a strong neuroinflammatory component contributing to neuronal death and brain atrophy (Schwab, C. & McGeer, P. L. “Inflammatory aspects of Alzheimer disease and other neurodegenerative disorders”, J. Alzheimers Dis., 2008; 13:359-369; Scheffer, S., et al., “Vascular Hypothesis of Alzheimer Disease: Topical Review of Mouse Models”, Arterioscler. Thromb. Vasc. Biol., 2021; 41:1265-1283).

Brain atrophy in AD involves multiple brain regions and begins relatively early in disease progression, affecting broad areas of the occipital, parietal, frontal, and temporal lobes, as well as the hippocampus, up to 8 years prior to AD diagnosis (Scahill, R. I., et al., “Mapping the evolution of regional atrophy in Alzheimer's disease: unbiased analysis of fluid-registered serial MRI”, Proc. Natl. Acad. Sci. USA, 2002; 99:4703-4707; Traini, E., et al., “Volume Analysis of Brain Cognitive Areas in Alzheimer's Disease: Interim 3-Year Results from the ASCOMALVA Trial”, J. Alzheimers Dis., 2020; 76:317-329; Apostolova, L. G., et al., “Conversion of mild cognitive impairment to Alzheimer disease predicted by hippocampal atrophy maps”, Arch. Neurol., 2006; 63:693-699; Jia, J., et al., “Biomarker Changes during 20 Years Preceding Alzheimer's Disease”, N. Engl. J. Med., 2024; 390:712-722). MRI imaging has revealed progressive atrophy at 39 weeks by volumetric magnetic resonance imaging (MRI), affecting multiple brain regions and whole brain volume, accompanied by increased ventricular size. During disease progression, biomarkers of brain atrophy may include decreases in the volume of whole brain, gray matter, temporal cortex, medial temporal cortex, hippocampus, frontal cortex, and/or thalamus, while volume increases may also be observed in the lateral ventricles, white matter, or cingulate cortex, as described in further detail below in this disclosure. Additionally, mean diffusivity or free water measurement in the cingulate cortex may increase with disease progression. These biomarkers can be assessed quantitatively, and over time during disease progression, using MRI.

Patients suffering from AD have been known to elicit irregular immune responses. Indeed, it has been shown that an abundance of pro-inflammatory cytokines exists in the vicinity of amyloid deposits and neurofibrillary tangles, thus hinting at an association between systemic inflammation and β-amyloid accumulation. Even in light of such evidence, those skilled in the art have repeatedly been skeptical of the immune system's role in the development of AD, since there has been no direct correlation between the inhibition of pro-inflammatory cytokines in humans and the reduction of AβP accumulation.

The use of a composition comprising allogeneic mesenchymal stem cells (MSCs) is able to combat the symptoms of AD. Treating a subject suffering from AD symptoms with a composition that includes allogeneic stem cells has been discovered to improve the subject's brain morphology and promote the expression of biomarkers that are associated with anti-inflammation and vascular repair. Allogeneic MSCs are also shown to be capable of promoting improvements in neuroinflammation and vascular function of a subject suffering from symptoms of AD. MSCs has been discovered to improve the subject's brain morphology and ameliorate brain atrophy, and promote the expression of novel quantitative biomarkers, including but not limited to the previously noted MRI biomarkers and blood serum biomarkers, for diagnosing and evaluating the progression of AD and the effectiveness of the treatment methods.

The above discoveries are surprising due to the ambiguity surrounding the pathogenesis of AD and the general reservation of those skilled in the art to use MSCs in treatments for AD since they were expected to perform poorly due to their inability to directly target beta-amyloids and their low residence time in the human body. They were also expected to perform poorly in AD treatments due to their large size, which led those skilled in the art to believe that they could not pass the blood-brain barrier (BBB) and migrate to/reach the site of inflammation and damage.

Another advantage of using MSCs in treatments for AD is that they may not involve targeting a single pathway or biomarker, such as without limitation AβP accumulation. Instead, the use of MSCs in AD treatments can allow multiple pathways to be targeted at once and thereby halt or significantly slow the progression of AD.

Following the discoveries discussed above, one aspect of the present application relates to methods of treating AD or alleviating the symptoms of AD, wherein the methods include administering to a subject suffering from symptoms of AD a composition including allogenic MSCs.

Another aspect of the present application relates to methods of treating HLHS, wherein the methods include administering to a subject suffering from HLHS a composition including allogenic MSCs.

Another aspect of the present application relates to providing novel biomarkers for diagnosing and evaluating the progression of AD and the effectiveness of the treatment methods associated thereto.

Another aspect of the present application relates to methods for evaluating potency of allogeneic MSCs using a potency assay.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (e.g., a step is performed by or with the assistance of a human).

As used herein, including the claims, the phrase “at least some” means “one or more” and includes the case of only one. Thus, e.g., the phrase “at least some ABCs” means “one or more ABCs” and includes the case of only one ABC.

As used herein, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

As used herein, the term “portion” means some or all. Therefore, for example, “a portion of X” may include some of “X” or all of “X”. In the context of a conversation, the term “portion” means some or all of the conversation.

As used herein, including the claims, the phrase “using” means “using at least” and is not exclusive. Thus, e.g., the phrase “using X” means “using at least X”. Unless specifically stated by use of the word “only”, the phrase “using X” does not mean “using only X”.

As used herein, including the claims, the phrase “based on” means “based in part on” or “based, at least in part, on” and is not exclusive. Thus, e.g., the phrase “based on factor X” means “based in part on factor X” or “based, at least in part, on factor X”. Unless specifically stated by use of the word “only”, the phrase “based on X” does not mean “based only on X”.

In general, as used herein, including the claims, unless the word “only” is specifically used in a phrase, it should not be read into that phrase.

As used herein, including the claims, the phrase “distinct” means “at least partially distinct”. Unless specifically stated, distinct does not mean fully distinct. Thus, e.g., the phrase “X is distinct from Y” means that “X is at least partially distinct from Y” and does not mean that “X is fully distinct from Y”. Thus, as used herein, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

As used herein, including the claims, the terms “multiple” and “plurality” mean “two or more,” and include the case of “two”. Thus, e.g., the phrase “multiple ABCs” means “two or more ABCs” and includes “two ABCs”. Similarly, e.g., the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, e.g., “about 3” or “approximately 3” shall also cover exactly 3, and “substantially constant” shall also cover exactly constant.

As used herein, unless stated otherwise, the terms “about” or “approximately” refer to a value that is within 10% above or below the value being described.

As used herein, including the claims, singular forms of terms are to be construed as also including the plural form and vice versa, unless the context indicates otherwise. Thus, it should be noted that as used herein, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

As used herein, the terms “administration” or “administering” refer to a method of giving a dosage of a compound or pharmaceutical composition to a subject. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

As used herein, generally speaking, an “effective amount” or “therapeutically effective amount” is the amount of a composition of this disclosure which, when administered to a subject, is sufficient to effect treatment of a disease or condition in the subject. The amount of a composition of this disclosure which constitutes a “therapeutically effective amount” may vary depending on the composition, the condition and its severity, the manner of administration, and the age of the subject to be treated.

As used herein, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of a neurological disorder). The term “therapeutic” includes the effect of mitigating deleterious clinical effects of certain processes (i.e., consequences of the process, rather than the symptoms of processes). As nonlimiting examples, a treatment may include (i) preventing a disease or condition from occurring in a subject, in particular, when such subject is predisposed to the condition but has not yet been diagnosed as having it; (ii) inhibiting a disease or condition, i.e., arresting its development; (iii) relieving a disease or condition, i.e., causing regression of the disease or condition; or (iv) relieving the symptoms resulting from a disease or condition, i.e., relieving pain without addressing the underlying disease or condition.

As used herein, a “subject” includes, but is not limited to, humans and non-human vertebrates such as wild, domestic, and farm animals. The terms “subject” and “patient” may be used interchangeably throughout this disclosure.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.,”), and the like, is merely intended to better illustrate the invention and does not indicate a limitation on the scope of the invention unless specifically so claimed.

While the invention has been described in connection with what is presently considered to be the most practical and embodiments thereof are further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

### Treatment of AD or HLHS Using MSCs

In some embodiments, the invention described herein may be directed towards methods for alleviating the symptoms of AD or HLHS in a subject in need thereof. In some embodiments, the invention described herein may be directed towards methods for treatment of AD or inhibition of AD disease progression. The method may include administering to the subject a composition including a therapeutically effective amount of allogeneic MSCs. As used herein, a “therapeutic amount” is an amount of a composition of matter that, once administered to a subject, is sufficient to treat Alzheimer's disease (AD) or hypoplastic left heart syndrome (HLHS), alleviate one or more symptoms of AD or HLHS, and/or inhibit the progression of AD or HLHS in the subject. As used herein, an “allogeneic” cell is a cell that is of the same animal species as, but genetically different in one or more genetic loci from, an animal that becomes a recipient host. This usually applies to cells transplanted from one animal to another non-identical animal of the same species.

In some embodiments, the MSCs used in the methods for treatment of AD or HLHS may include Lomecel-B™. As used herein, “Lomecel-B” is a type of living human cell derived from MSCs. Lomecel-B™ may be isolated from fresh bone marrow tissue that has been donated by adult donors aged 18 to 45. Because the cells come from another individual, Lomecel-B™ may be referred to as an “allogeneic” (donor-derived) product, consistent with details described elsewhere in this disclosure. Once the MSCs have been isolated from the fresh bone marrow through a careful selection process, the cells are culture-expanded, i.e., allowed to replicate under controlled laboratory conditions, into the billions using specialized techniques and processes.

After a specific number of expansion cycles called “passages”, the cells may be harvested, separated into specific doses (e.g. 50 million cells), and frozen until future use. Lomecel-B™ may exert its therapeutic effects through a variety of mechanisms, such as without limitation by creating pro-vascular and/or immunomodulatory effects, among others. Specifically, Lomecel-B™ secretes multiple factors that modulate important pro-vascular and anti-inflammatory pathways. In vivo and in vitro studies reveal that these effects can be mediated, at least in part, via the tyrosine kinase with immunoglobulin and epidermal growth factor homology domains (TIE2) signaling pathway. Lomecel-B™ may strongly express vascular endothelial growth factor A (VEGF-A), which is a key promoter of angiogenesis. Lomecel-B™ demonstrated a lack of deterioration, a prevention of disease worsening based on cognitive assessment, and an improvement of quality of life based on caregiver assessment. Additional details will be provided below in this disclosure.

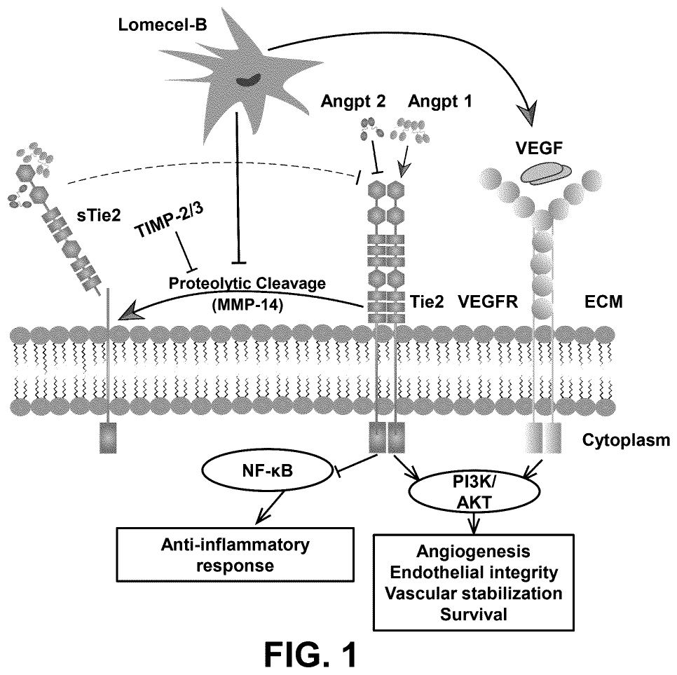

FIG. 1 depicts mechanisms of actions (MOAs) of Lomecel-B™ cells for treatment of Alzheimer's disease and/or alleviation of symptoms thereof. TIE2, a cognate, cell surface receptor for angiopoietins 1 and 2, is expressed by endothelial cells. TIE2 activates pro-angiogenic and anti-inflammatory downstream signaling pathways (Sato, T. N., et al., “Distinct roles of the receptor tyrosine kinases Tie-1 and Tie-2 in blood vessel formation”, Nature, 1995; 376:70-74). Downstream signaling of TIE2 converges with vascular endothelial growth factor R (VEGF-R) activation. These pathways activate PI3 kinase (PI3K)/AKT pro-angiogenic signaling and inhibit NF-kB inflammatory responses. The TIE2 pathway is an important regulator of vascular and inflammatory responses. Under certain pathological conditions, TIE2 can be degraded, such as without limitation by matrix metalloproteinase-14 (MMP-14) activity, into a soluble form, i.e., soluble TIE2 (sTIE2). STIE2 is subsequently released into the bloodstream and detected in circulation (Idowu, T. O., et al., “Identification of specific Tie2 cleavage sites and therapeutic modulation in experimental sepsis”, Elife, 2020:9: e59520). The concentration of sTIE2 was recently found to increase in the blood serum in AD patients indicating cell-surface receptor shedding and therefore inactivation that would reduce anti-inflammatory activity. In preferred embodiments, the level of sTIE2 in the blood serum or blood plasma may be reduced after administration of allogenic MSCs to a subject. Additional details will be provided below in this disclosure.

In some embodiments, the composition for treatment of AD or HLHS is configured to perform one or more therapeutic functions by preventing TIE2 from degradation. As a nonlimiting example, MMP-14 may be inhibited by tissue-inhibitor-of-metalloprotease-2 (TIMP2), a secreted protein; accordingly, the therapeutic mechanism of the composition and/or MSCs (e.g., Lomecel-B™) for treatment of AD may include protection of TIE2. As a nonlimiting example, protection of TIE2 via TIMP2/MMP-14 inhibition may represent a unique therapeutic pathway for Lomecel-B™, as described in further detail below in this disclosure. It is worth noting that such mechanism may offer benefits that extend beyond treatment of AD, and may be implemented for treatment of, for example and without limitation, hypoplastic left heart syndrome (HLHS). Additionally, and/or alternatively, Lomecel-B™ may exert anti-inflammatory therapeutic effects both through TIE-2 protection and through the IL-6/8 pathway. IL-6, and IL-8 are also highly expressed by Lomecel-B™ and may be cooperatively involved in suppressing inflammation.

The composition for treatment of AD or HLHS may include any dosage of MSCs deemed suitable by a person of ordinary skill in the art, upon reviewing the entirety of this disclosure. In some embodiments, the composition for treatment of AD or HLHS may include a dosage of approximately 20×10⁶ MSCs. In some embodiments, the composition may include a dosage of approximately 100×10⁶ MSCs. In some embodiments, the composition may include a dosage of any amount between 20×10⁴ and 100×10⁸ MSCs. As nonlimiting examples, the composition may include a dosage of approximately 25×10⁴, approximately 30×10⁴, approximately 35×10⁴, approximately 40×10⁴, approximately 45×10⁴, approximately 50×10⁴, approximately 55×10⁴, approximately 60×10⁴, approximately 65×10⁴, approximately 70×10⁴, approximately 75×10⁴, approximately 80×10⁴, approximately 85×10⁴, approximately 90×10⁴, approximately 95×10⁴, approximately 5×10⁶, approximately 10×10⁶, approximately 15×10⁶, approximately 20×10⁶, 25×10⁶, approximately 30×10⁶, approximately 35×10⁶, approximately 40×10⁶, approximately 45×10⁶, approximately 50×10⁶, approximately 55×10⁶, approximately 60×10⁶, approximately 65×10⁶, approximately 70×10⁶, approximately 75×10⁶, approximately 80×10⁶, approximately 85×10⁶, approximately 90×10⁶, approximately 95×10⁶, approximately 5×10⁸, approximately 10×10⁸, approximately 15×10⁸, approximately 20×10⁸, approximately 25×10⁸, approximately 50×10⁸, approximately 100×10⁸, and/or the like. As further nonlimiting examples, the composition may include a dosage of between 20×10⁴ and 20×10⁵ MSCs, between 20×10⁵ and 20×10⁶ MSCs, between 20×10⁶ and 25×10⁶ MSCs, between 25×10⁶ and 30×10⁶ MSCs, between 30×10⁶ and 35×10⁶ MSCs, between 35×10⁶ and 40×10⁶ MSCs, between 40×10⁶ and 45×10⁶ MSCs, between 45×10⁶ and 50×10⁶ MSCs, between 50×10⁶ and 55×10⁶ MSCs, between 55×10⁶ and 60×10⁶ MSCs, between 60×10⁶ and 65×10⁶ MSCs, between 65×10⁶ and 70×10⁶ MSCs, between 70×10⁶ and 75×10⁶ MSCs, between 75×10⁶ and 80×10⁶ MSCs, between 80×10⁶ and 85×10⁶ MSCs, between 85×10⁶ and 90×10⁶ MSCs, between 90×10⁶ and 95×10⁶ MSCs, between 95×10⁶ and 100×10⁶ MSCs, between 100×10⁶ and 100×10⁷ MSCs, or between 100×10⁷ and 100×10⁸ MSCs. Additionally, and/or alternatively, in some embodiments, the composition for treatment of AD may include allogeneic human microglial cells (HMCs).

In some embodiments, the composition including MSCs for treatment of AD or HLHS may be administered to a subject in need thereof by intravenous or intra-arterial infusion.

In some embodiments, the composition including MSCs for treatment of AD or HLHS may be administered monthly.

In some embodiments, the composition including MSCs for treatment of AD or HLHS may be administered to a subject in need thereof as a single dose, a monthly dose, and/or a repeated interval dose.

Subjects who underwent treatment of AD using the composition including MSCs showed no AD-related imaging abnormalities (ARIA-E, ARIA-H, etc.), infusion-related reactions, or interruptions of infusions. No subject died from the treatment.

An exemplary study of using MSCs to treat AD and evaluating the efficacy thereof follows the following protocol. Unless noted otherwise, all experimental data were collected based on such protocol.

Brain MRI was performed on a population of subjects at screening, as well as by Weeks 13, 26, 39, and 52, to assess for safety (including ARIA), and was further used for evaluating structural brain changes.

The population of subjects for the trials was 60-85 years of age, having a Mini Mental State Exam (MMSE) score between 18-24, with a diagnosis of mild Alzheimer's disease. These subjects underwent a confirmatory positron emission tomography (PET) scan showing Alzheimer's disease as well as a Brain MRI ruling out any other exclusionary criteria.

Four infusions, namely Infusion 1, 2, 3, and 4, were performed to the subjects. Infusion 1 occurred at Day 0, Infusion 2 occurred during Week 4, Infusion 3 occurred during Week 8, and Infusion 4 occurred during week 12. All infusions were IV infusions of 80 mL Lomecel-B™ administered over 40 minutes at an infusion rate of 2 mL/min. Subjects had no pre or co medications.

Subjects were randomized into 4 groups of equal sizes. Specifically, Group 1 was infused with placebo 4 times for Infusions 1-4; Group 2 was treated with a low-dose composition including 25 million Lomecel-B™ for Infusion 1, followed by placebo at Infusions 2-4; Group 3 was treated with low-dose compositions each including 25 million Lomecel-B™ for Infusion 1-4; Group 4 was treated with high-dose compositions each including 100 million Lomecel-B™ for Infusion 1-4.

Brain volumetry was performed via MRI at Screening and Weeks 16, 26, and 39, to assess for volumetric changes in the hippocampus, overall brain size, ventricular volume, and/or other brain structures, each normalized for intracranial volume. Brain MRIs were performed using 3T (Tesla) scanners, 2 imaging centers were used by the 10 clinical centers, and one MRI scanner at each location was used for the entire trial.

In some embodiments, the method for treatment of AD, as described above, may further include examining a cerebral spinal fluid of the subject before and after administration of the composition comprising the therapeutically effective amount of the allogenic MSCs.

In some embodiments, the method for treatment of AD, as described above, may include examining the cerebral spinal fluid of a subject before and after administration of the compositions including allogeneic MSCs.

In some embodiments, the method for treatment of AD or HLHS, as described above, may include examining the blood serum of a subject before and after administration of the compositions including allogeneic MSCs.

In some embodiments, the method for treatment of AD or HLHS, as described above, may include examining the blood plasma of a subject before and after administration of the compositions including allogeneic MSCs.

In some embodiments, the method for treatment of AD, as described above, may further include determining if a change in the cortical amygdaloid transition area of the subject has occurred after administration of the composition including the therapeutically effective amount of the allogenic MSCs.

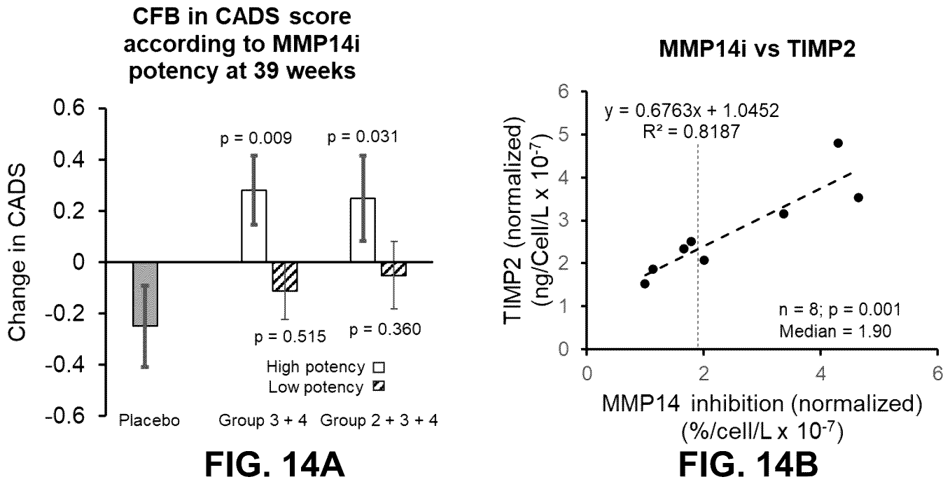

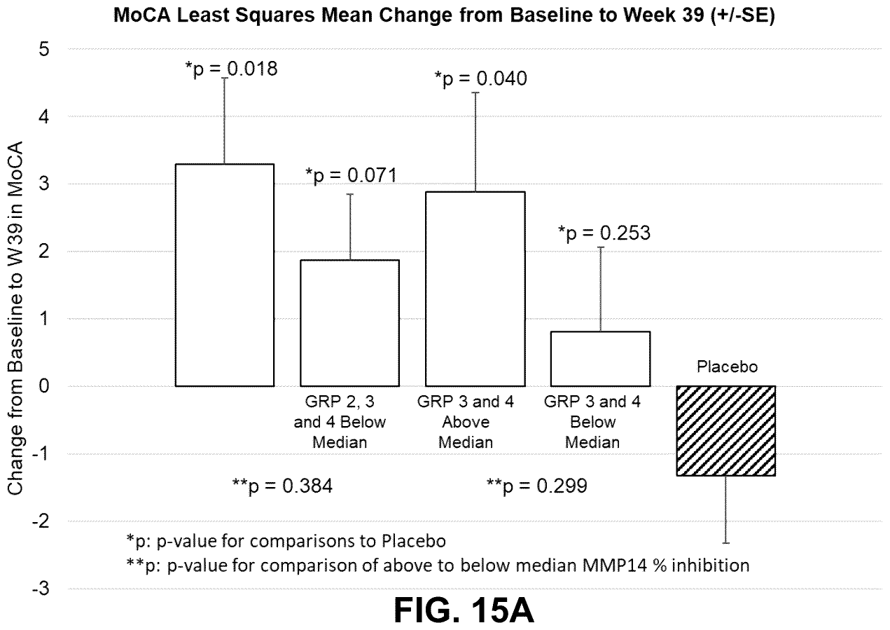

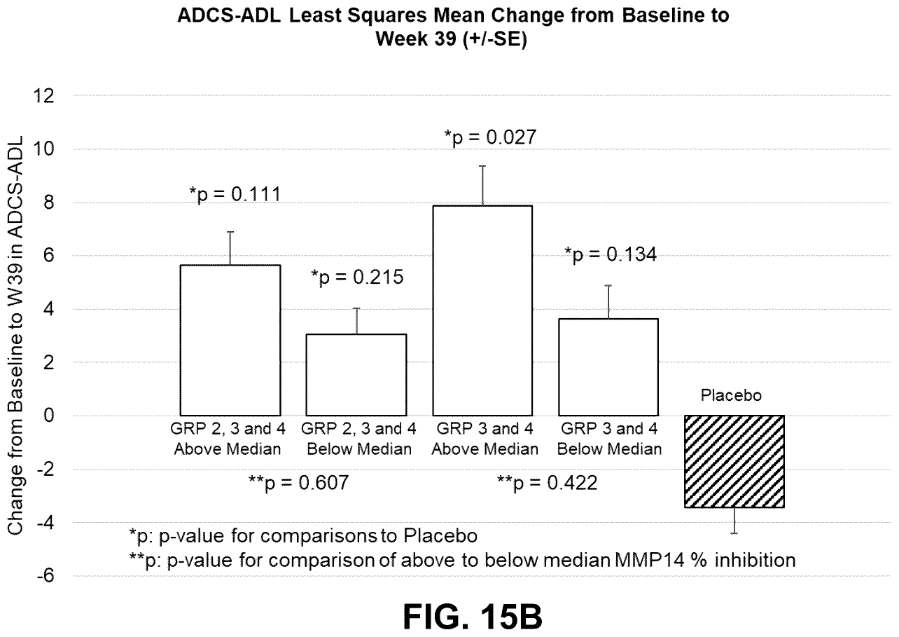

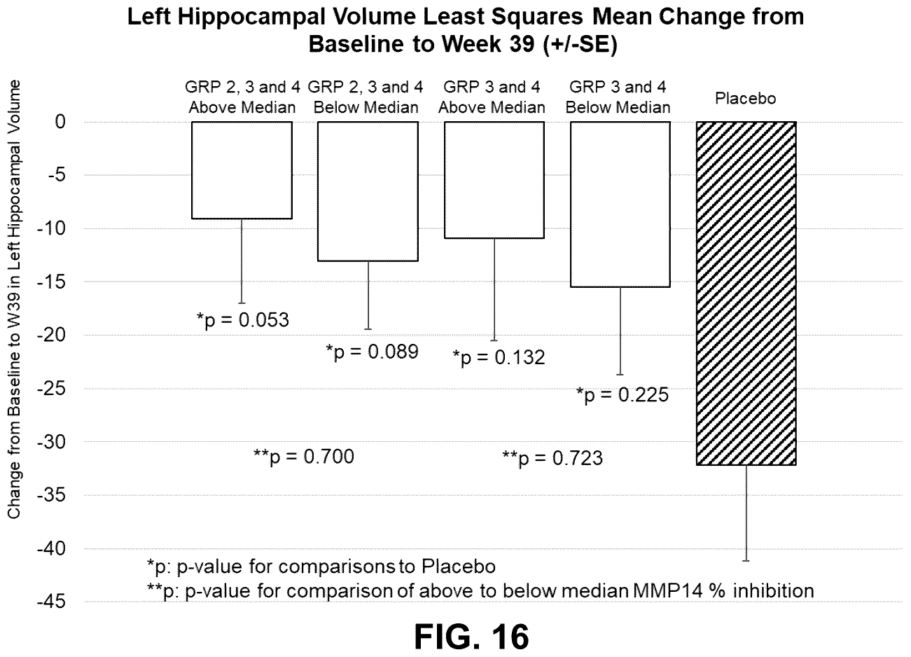

In some embodiments, the method for treatment of AD, as described above, may further include measuring a cognitive or quality-of-life function of the subject suffering from symptoms of AD before and after administration of the composition including the therapeutically effective amount of the allogenic MSCs. As nonlimiting examples, the cognitive function may be measured using Composite Alzheimer's Disease Score (CADS, FIG. 14A), Montreal Cognitive Assessment (MoCA, FIG. 15A), Alzheimer's Disease cooperative study—Activities of Daily Living Scale (ADCS-ADL, FIG. 15B), Mini-Mental State Examination (MMSE), and/or the like. High MMP-14i potency in general correlates with improved responses from subjects. Subjects who received above-median MMP-14i potency lots of Lomecel-B™ superior performance in CADS, MoCA, and ADCS-ADL than those who received below-median MMP-14i potency lots of Lomecel-B™. Additional details will be provided below in this disclosure.

In some embodiments, the method for treatment of AD or HLHS, as described above, may further include evaluating one or more biomarkers in the subject suffering from symptoms of AD before and after administration of the composition including the therapeutically effective amount of the allogenic MSCs. As used herein, a “biomarker” is an indication that signifies a normal or abnormal physiological process and/or marks a certain condition or disease. In some cases, a biomarker may be used to evaluate how well a subject responds to a treatment for a certain disease or condition. In some cases, a biomarker may include a detected presence of, a measured amount or concentration of, and/or another qualitative or quantitative metric based on one or more chemical/biochemical species. In some cases, a biomarker may include a one or more of a whole-blood, blood-plasma, or blood-serum biomarker or biomarkers. Additional details will be provided below in this disclosure.

### Novel Biomarkers

The invention described herein is also directed towards novel biomarkers for diagnosing and evaluating the progression of AD or HLHS and the effectiveness of the treatment methods associated thereto.

In some embodiments, a biomarker may include one or more MRI biomarkers. As used herein, an “MRI biomarker” is a measurable characteristic derived from a magnetic resonance imaging (MRI) scan that indicates the health of biological tissue. In some cases, MRI biomarkers can be used to diagnose tumors, monitor treatment response, and/or personalize cancer care.

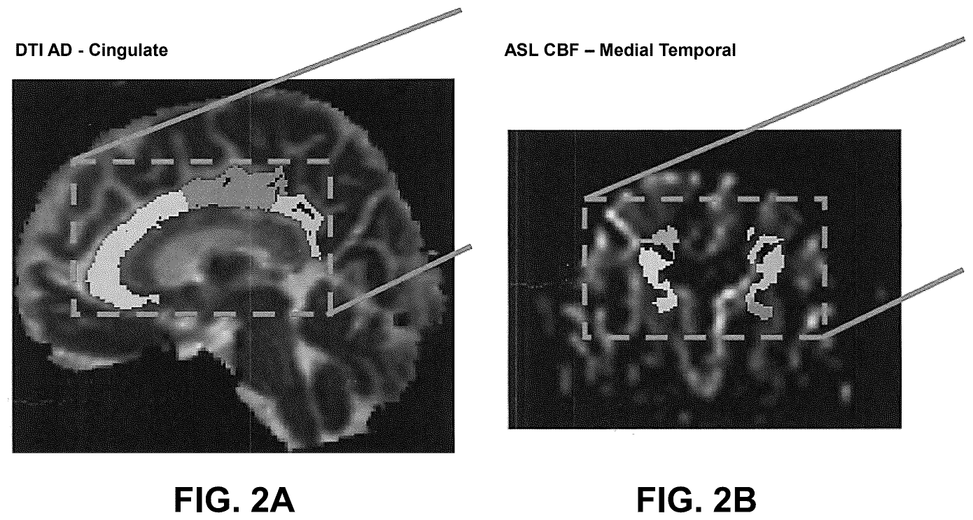

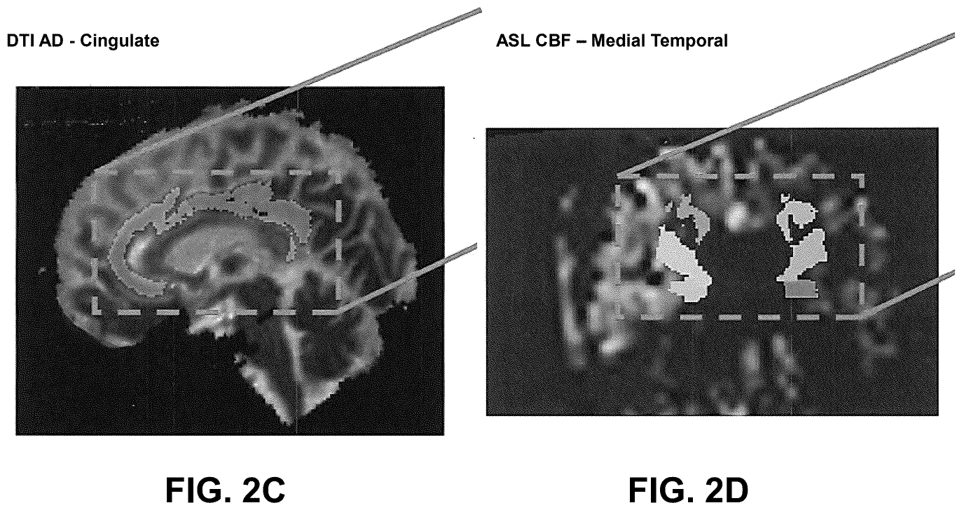

In some embodiments, evaluating the one or more MRI biomarkers may include evaluating the one or more MRI biomarkers using diffusion tensor imaging (DTI). As used herein, “diffusion tensor imaging” or “DTI” is a type of MRI scan that measures the movement of water molecules in the brain to create detailed images of the brain's nerve tracts. DTI is a noninvasive technique that uses radio waves and a magnetic field to produce images. DTI may be used to index tissue structure and neuroinflammation and has been a valuable tool in assessing AD brain pathology (Carlson, M. L., et al., “Hippocampal subfield imaging and fractional anisotropy show parallel changes in Alzheimer's disease tau progression using simultaneous tau-PET/MRI at 3T”, Alzheimer's & Dementia: Diagnosis, Assessment & Disease Monitoring (DADM), 2021; 13 (1): e12218). In the exemplary study described above, DTI via MRI was conducted at Screening and Weeks 16, 26, and 39 to assess for changes in neuroinflammation. FIGS. 2A and 2C are exemplary MR images of the cingulate cortex of subjects with or without MSC treatments, respectively, captured using DTI.

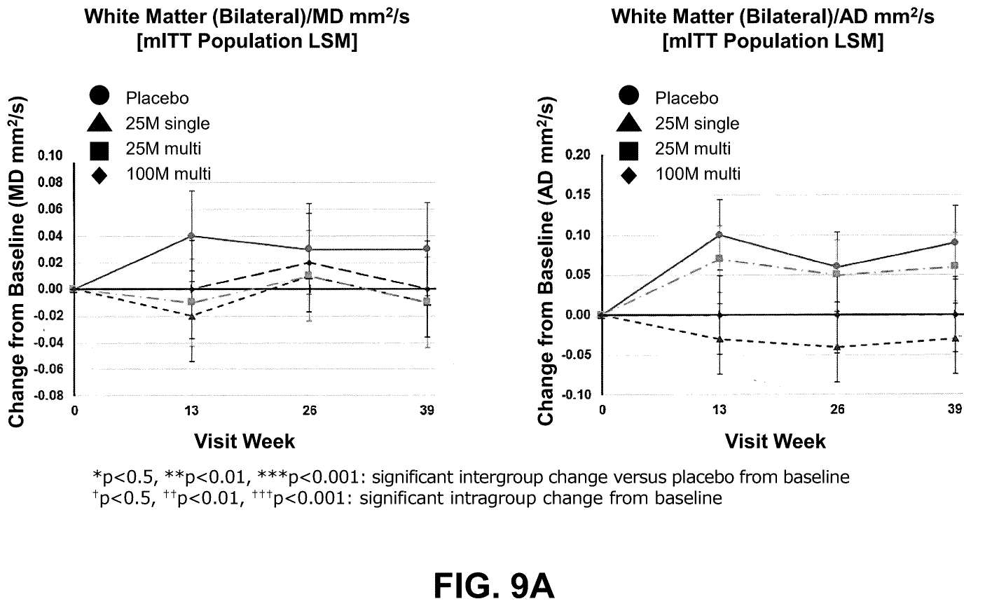

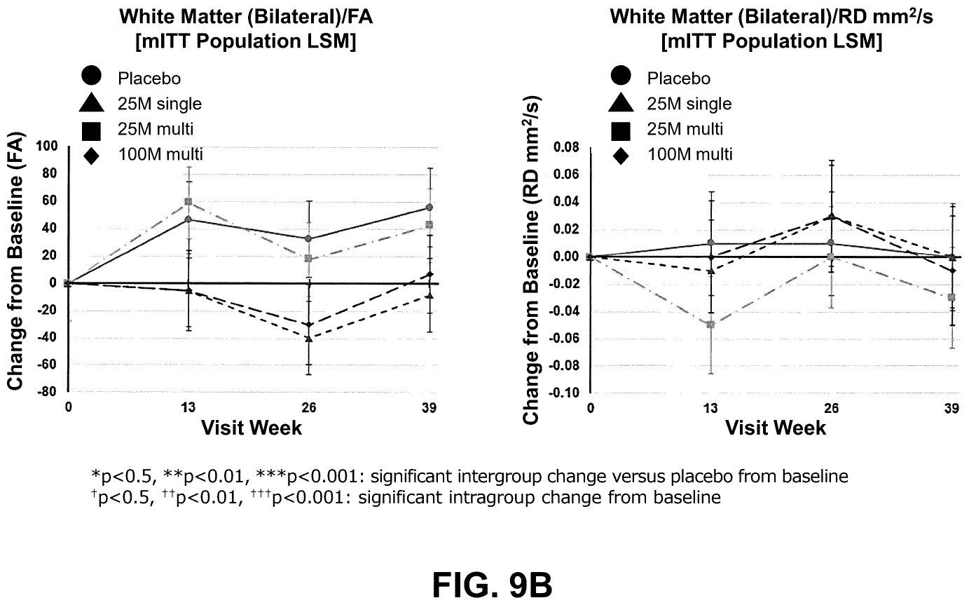

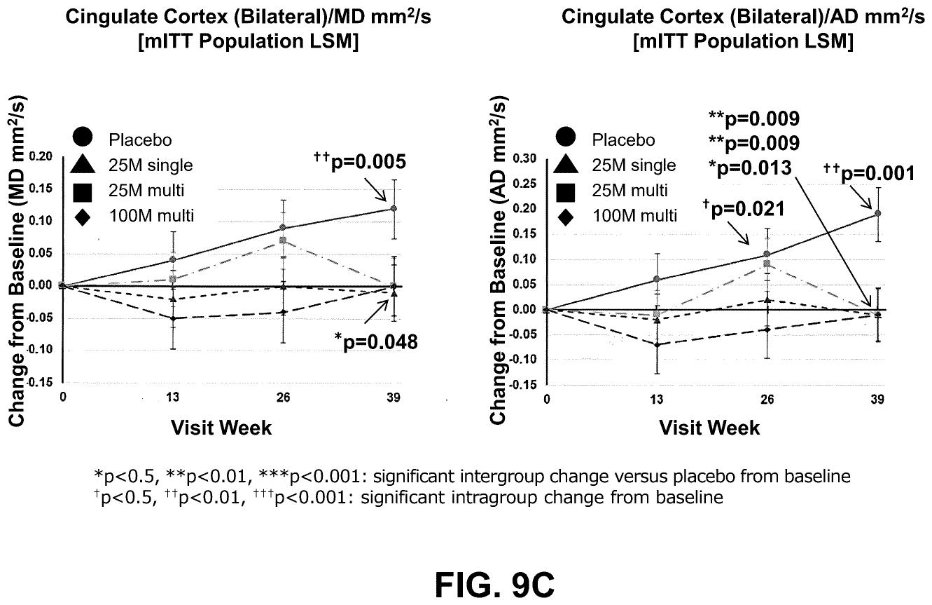

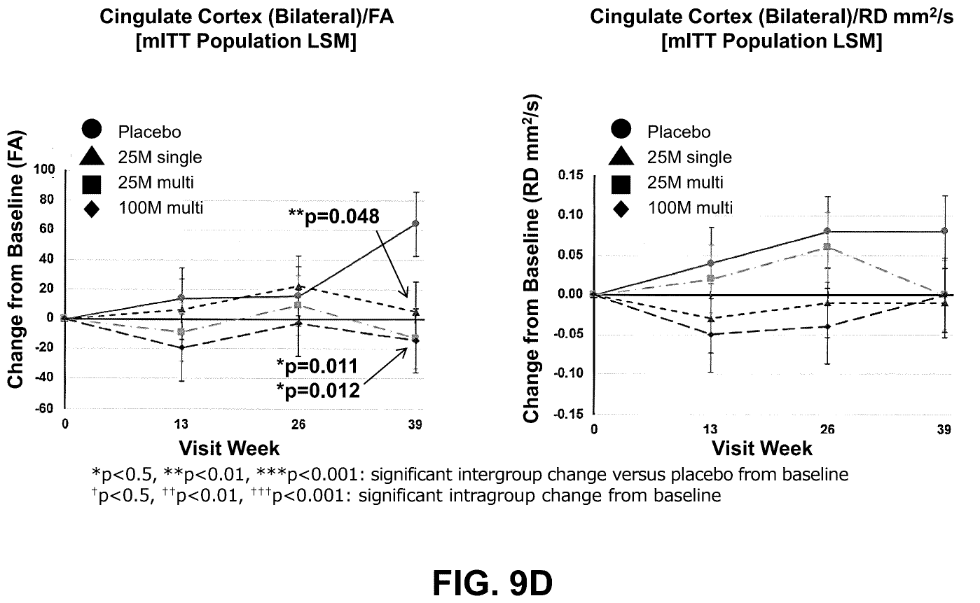

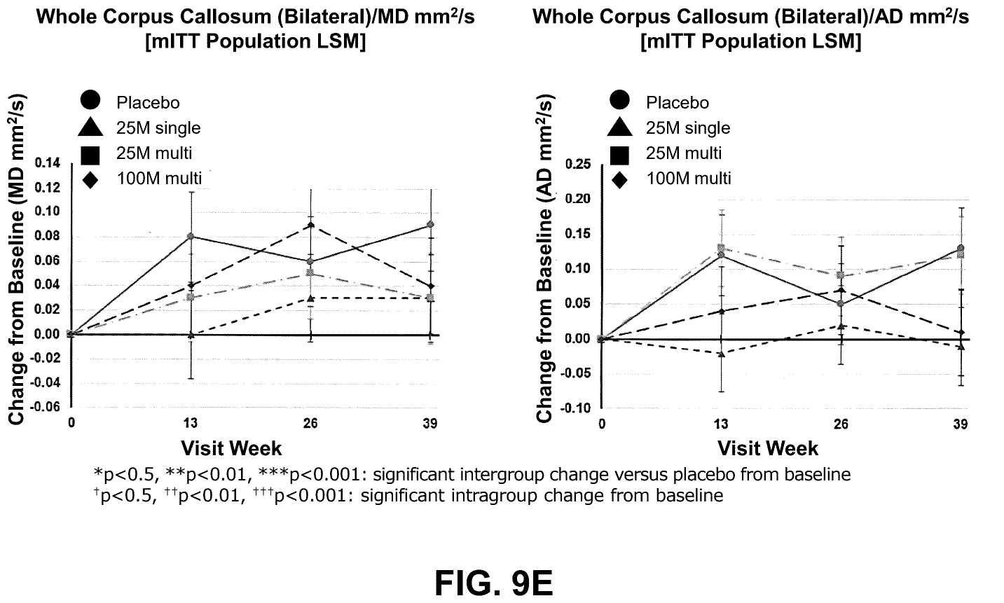

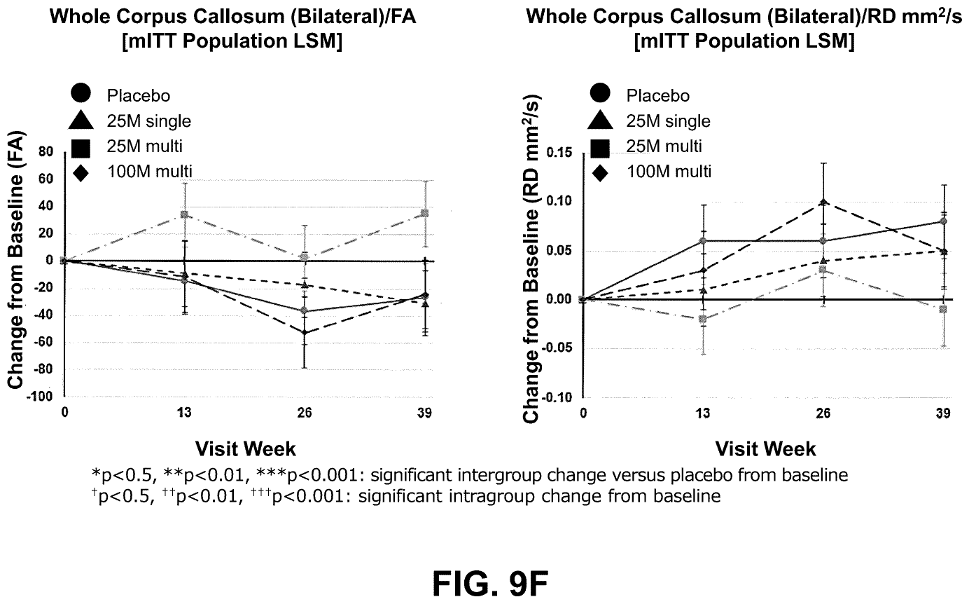

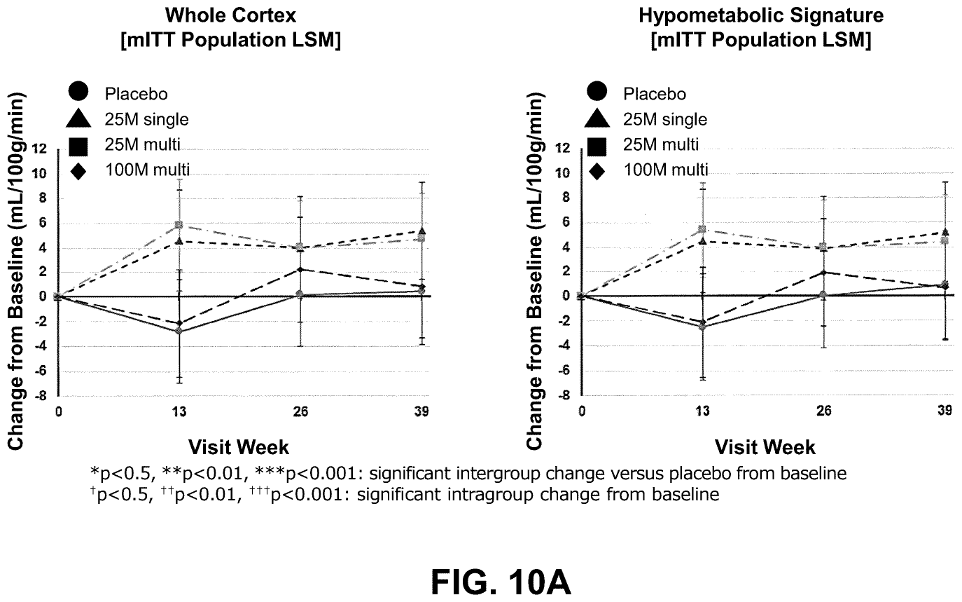

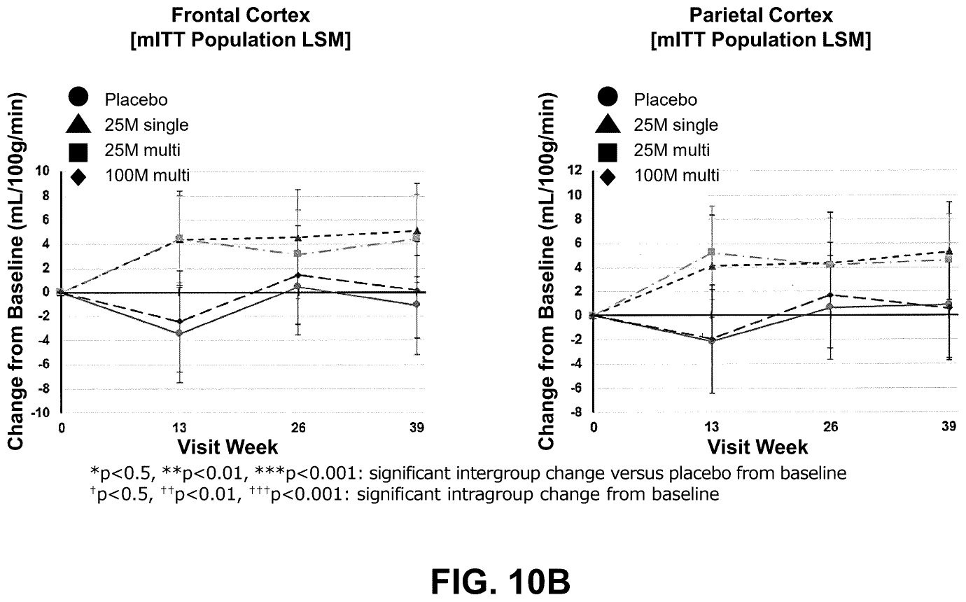

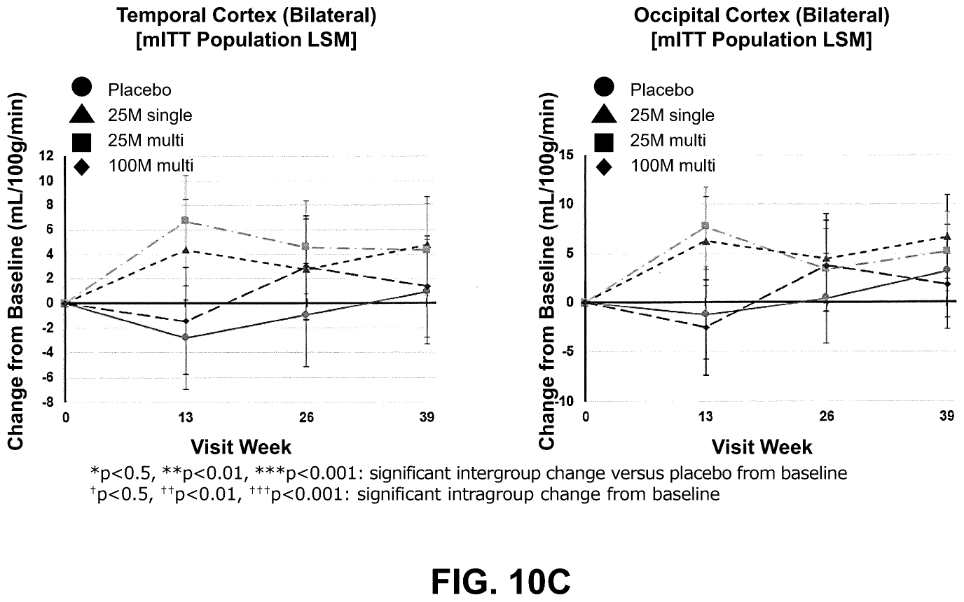

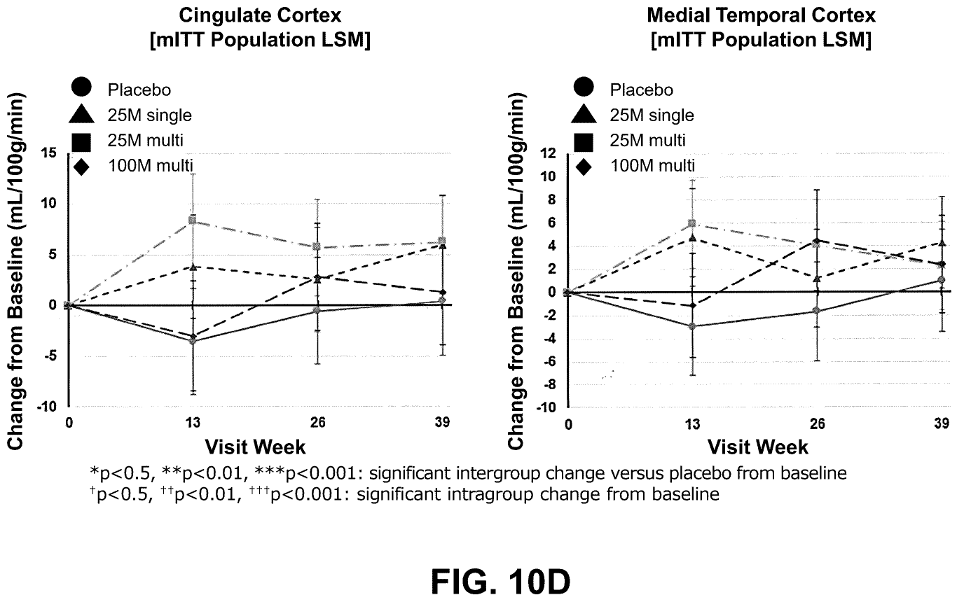

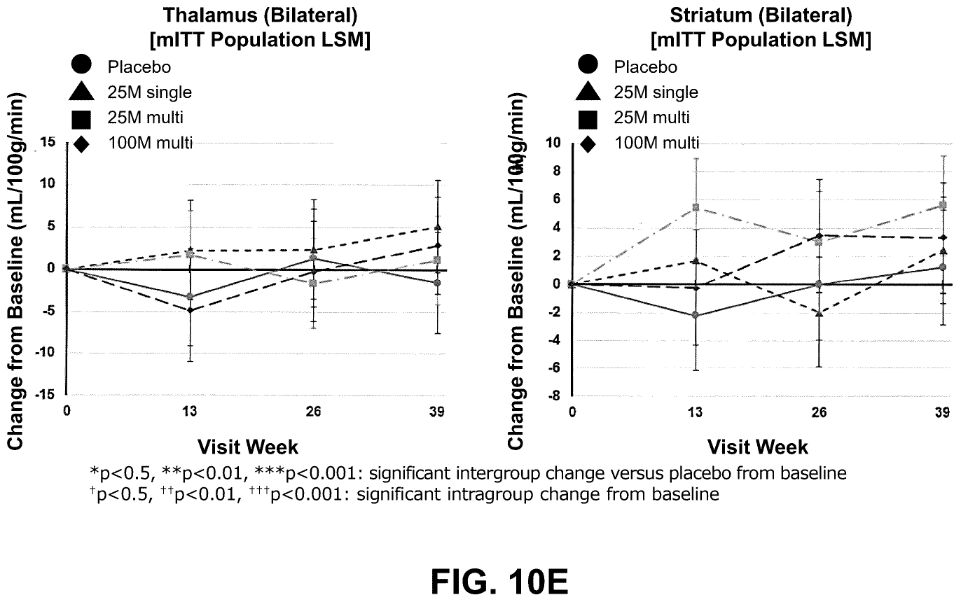

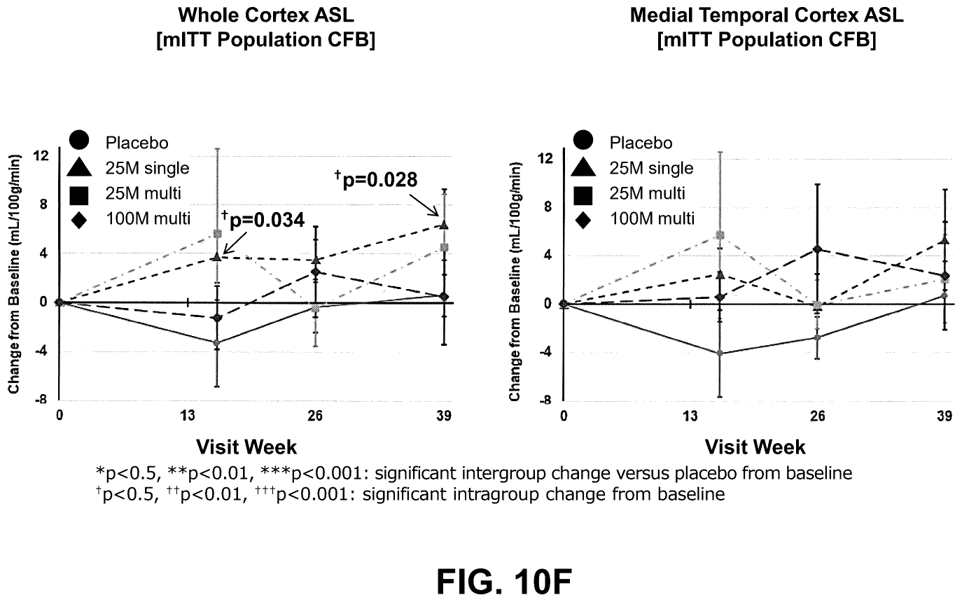

Various modalities of DTI and ASL measurements are shown in FIGS. 9A-F and FIGS. 10A-F. Specifically, DTI measurements were made under four modalities: mean diffusivity (MD), axial diffusivity (AD), fractional anisotropy (FA), and radial diffusivity (RD). While subjects without treatment showed a linear decrease, which is indicative of pathological damage, numerically superior results were achieved in Lomecel-B™ arms for both DTI-MD and DTI-AD (FIG. 9C).

In some embodiments, evaluating the one or more MRI biomarkers may include evaluating the one or more MRI biomarkers using arterial spin labeling (ASL). As used herein, “arterial spin labeling” or “ASL” is a non-invasive technique used to measure cerebral blood flow or perfusion by magnetically labeling the water protons in arterial blood, allowing for visualization of tissue blood flow without the need for contrast injection. Essentially, ASL “tags” the blood as it flows through the brain to assess its perfusion levels. An increase in the signal intensity of ASL may correlate with an increased blood flow to the brain area. FIGS. 2B, 2D are exemplary MR images of cerebral blood flow (CBF) in the medial temporal cortex of subjects without treatment, captured using arterial spin labeling (ASL). Additionally, and/or alternatively, similar MRI techniques may be used for evaluation of MRI biomarkers.

In some embodiments, the one or more MRI biomarkers may be evaluated using a volume measurement (i.e., volumetry). Such volume measurements may include a volume measurement of a whole brain, or one or more subregions of a whole brain, including without limitation a lateral ventricle, grey matter, a medial temporal cortex, a hippocampus, a thalamus, white matter, and/or a cingulate cortex, among other anatomical structures/substructures such as without limitation amygdala, cortical nucleus, hippocampal subregions, and/or the cortical amygdaloid transition area, among others. A shrinkage in whole-brain volume, often referred to as brain atrophy, may be used as a key indicator of AD, as it signifies a progressive loss of brain cells and neural connections, which leads to cognitive decline. Lateral ventricles are large fluid-filled spaces within the brain, an enlargement of which correlates to a decreased cerebral volume. Grey matter is a tissue in the brain where neuron cell bodies and synapses are concentrated; accordingly, a decrease in volume of grey matter may indicate a loss of neurons and synaptic connections. Medial temporal cortex is a brain region containing major memory-forming structures (e.g., hippocampus and amygdala). A decrease in the medial temporal cortex (particularly the hippocampus within this region) is often considered a significant indicator of AD, as it is one of the earliest and most prominent areas affected by the disease, leading to memory impairments. Hippocampus is a brain structure essential for forming new factual memories and a major brain site of rapid neurogenesis alongside olfactory bulb. Thalamus is a deep brain structure that is the major information relay station for the cerebral cortex.

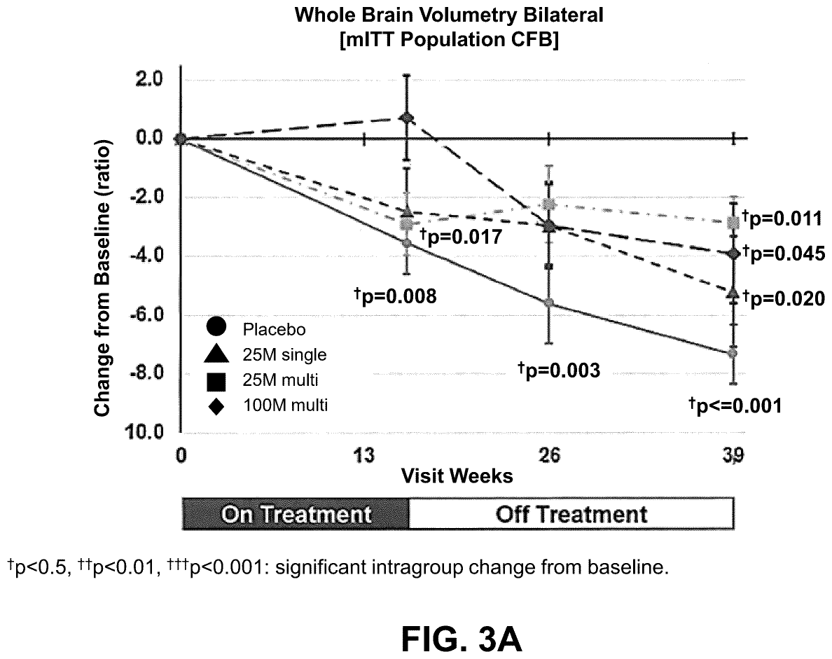

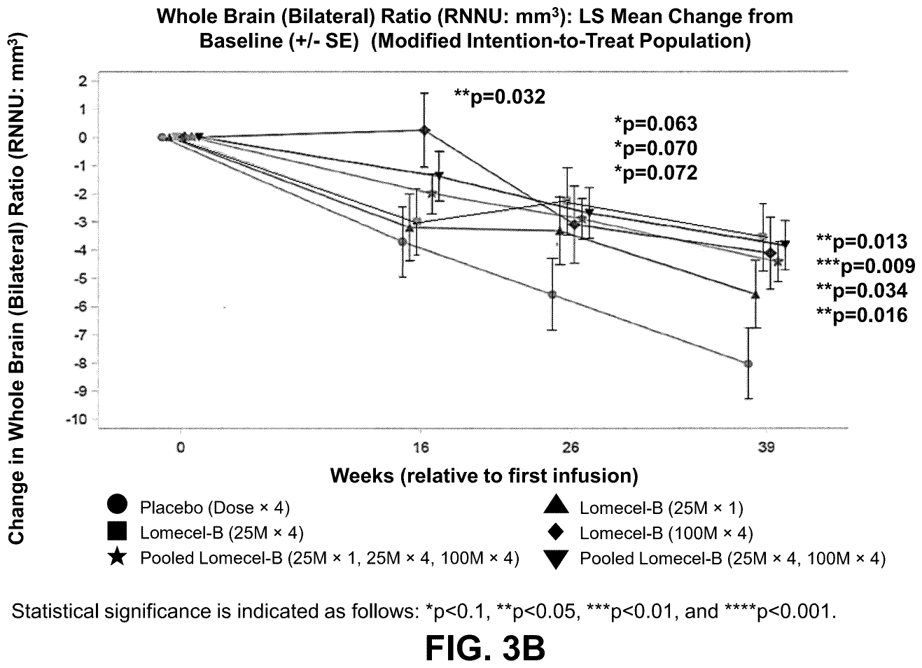

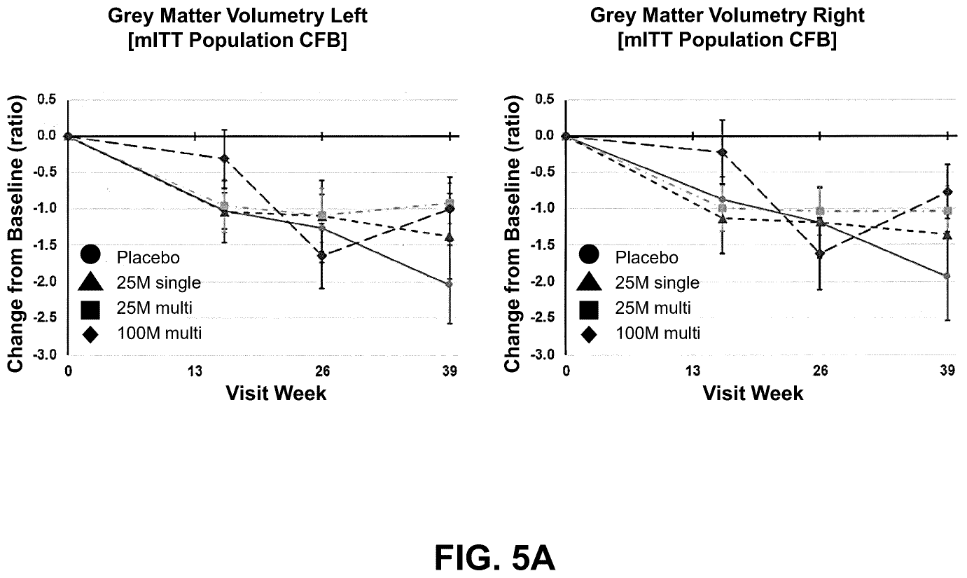

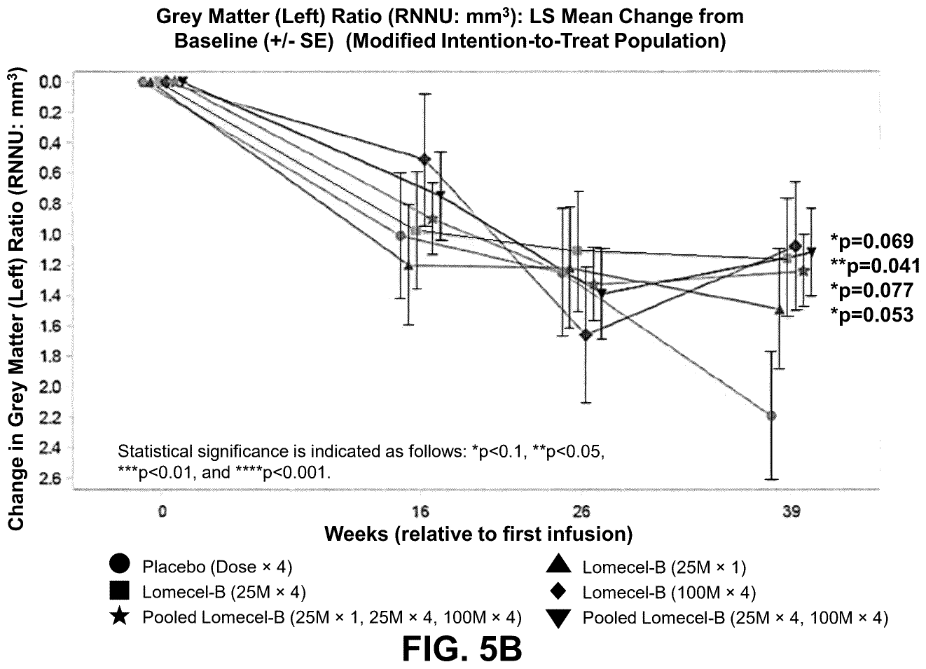

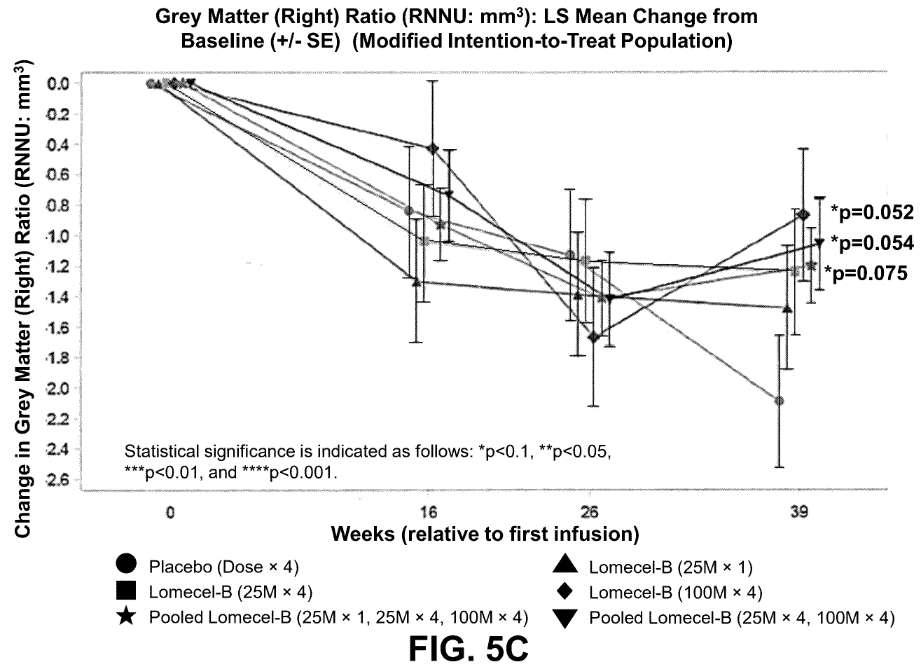

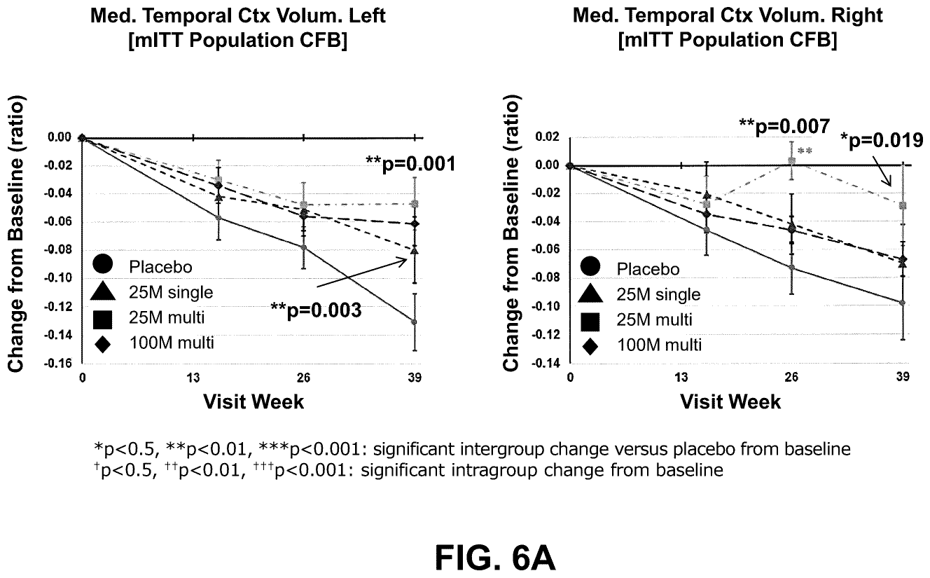

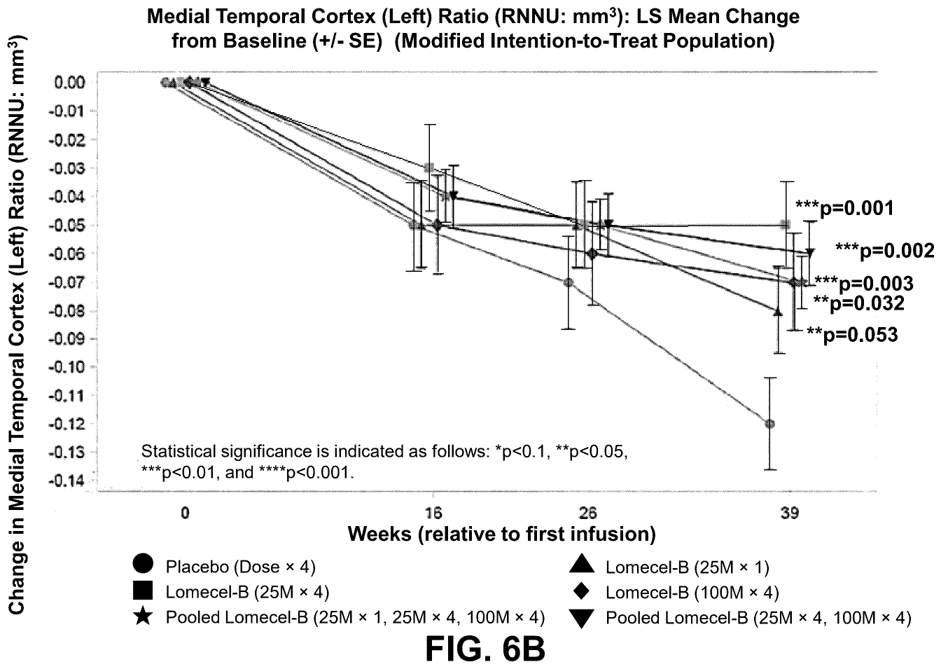

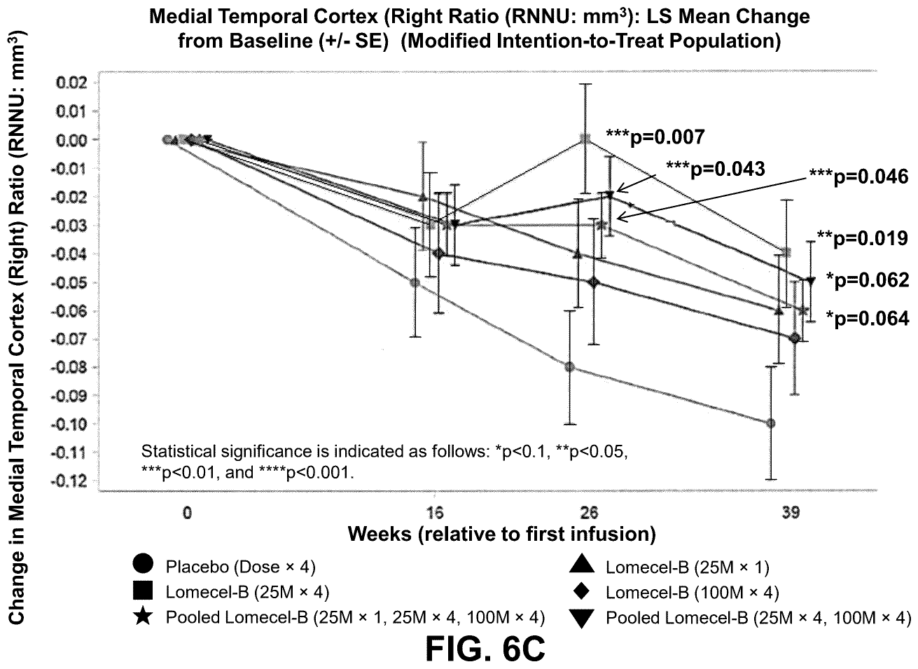

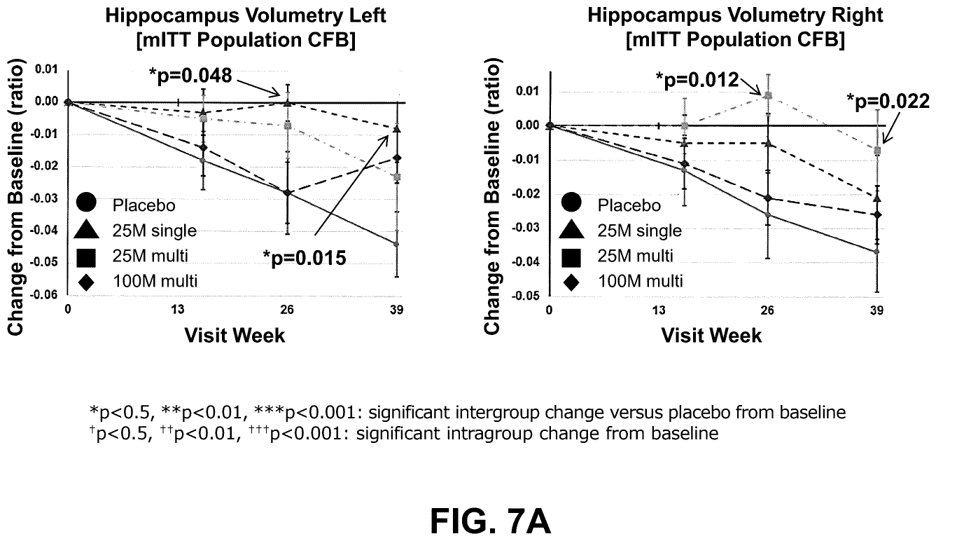

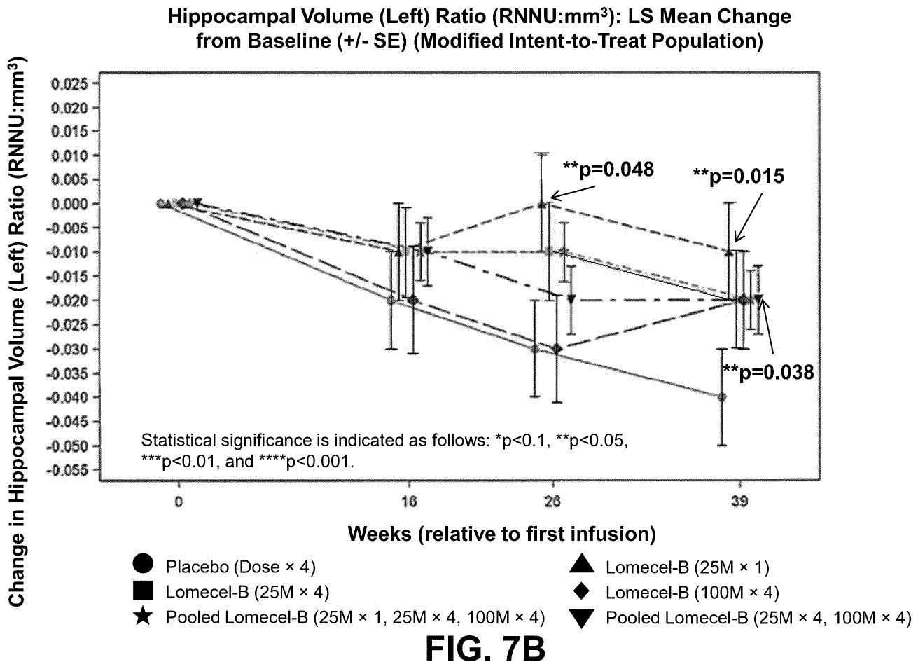

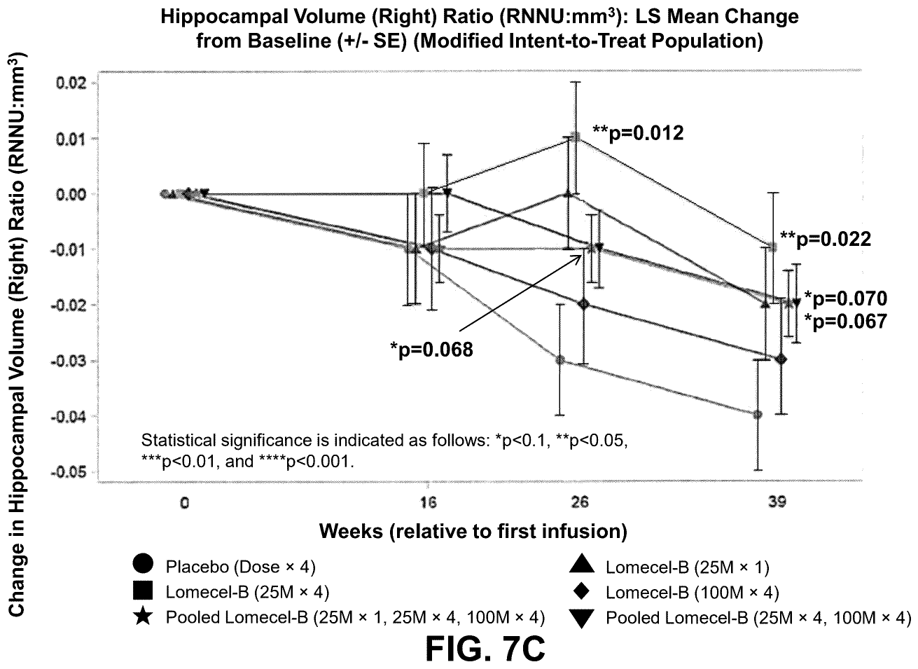

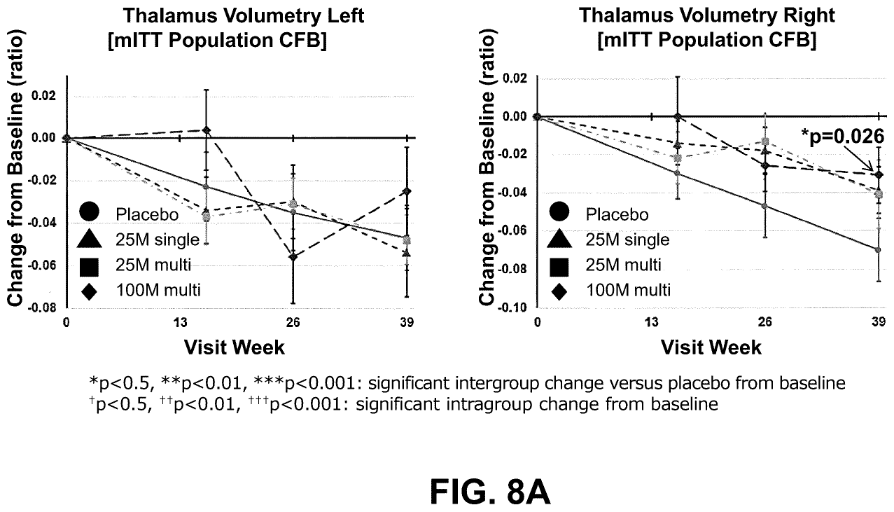

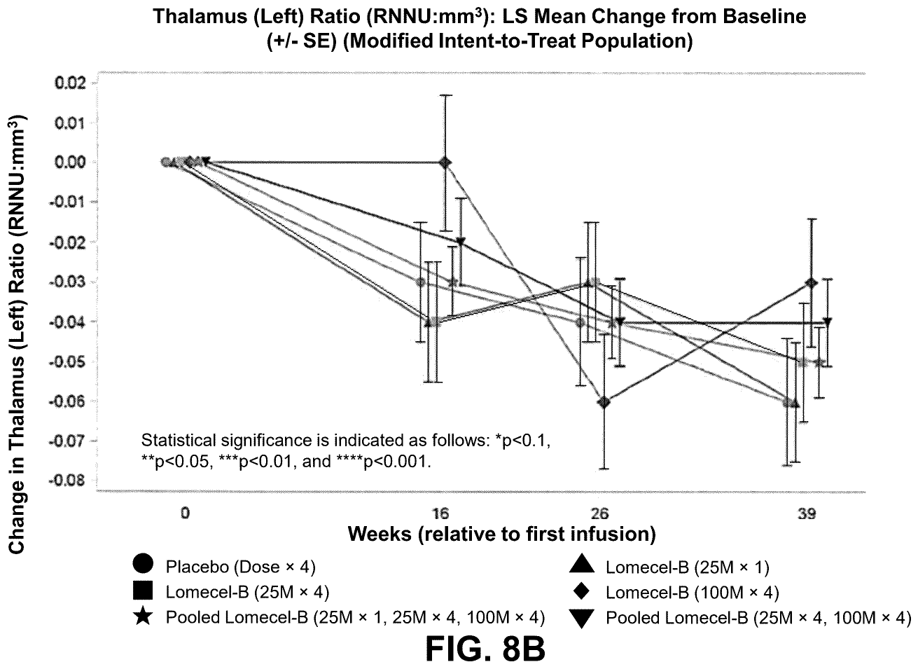

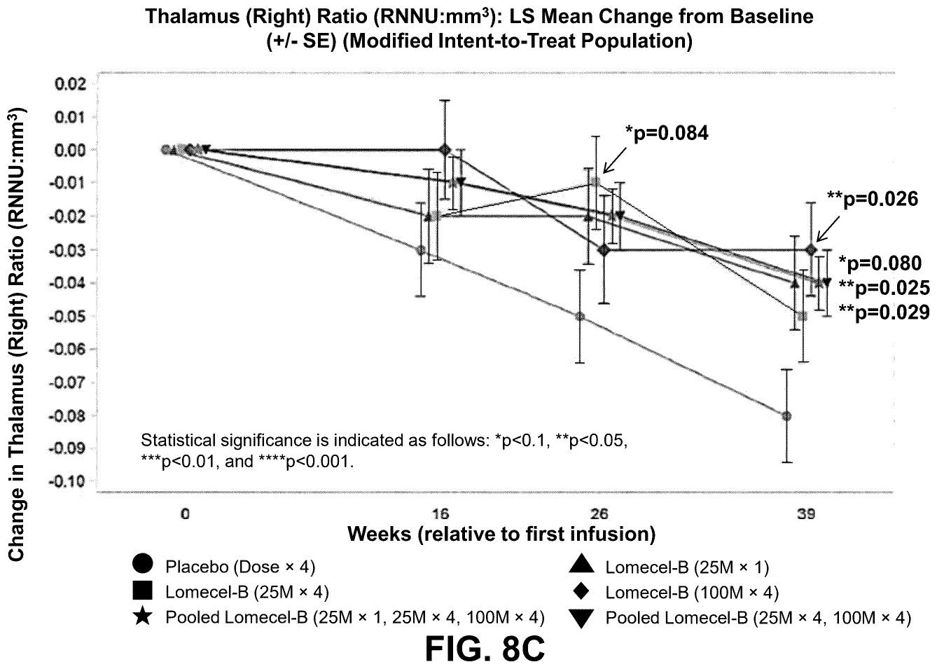

In some cases, the volume measurement may indicate a suppressed decrease in volume. In some cases, the suppressed decrease in volume may pertain to a whole brain (see FIGS. 3A-B), grey matter (FIGS. 5A-C), a medial temporal cortex (FIGS. 6A-C), a hippocampus (FIGS. 7A-C, 16), a thalamus (FIGS. 8A-C), white matter (FIGS. 9A-B), a cingulate cortex (FIGS. 9C-D), and/or the like, of a subject in need thereof after administration of the composition including the therapeutically effective amount of the allogenic MSCs. In the exemplary study described above, brain volumes for subjects treated with Lomecel-B™ (Groups 2-4) showed a decrease in decline relative to a linear decline observed among subjects without such treatment (Group 1, placebo). As nonlimiting examples, the suppressed decrease in volume can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

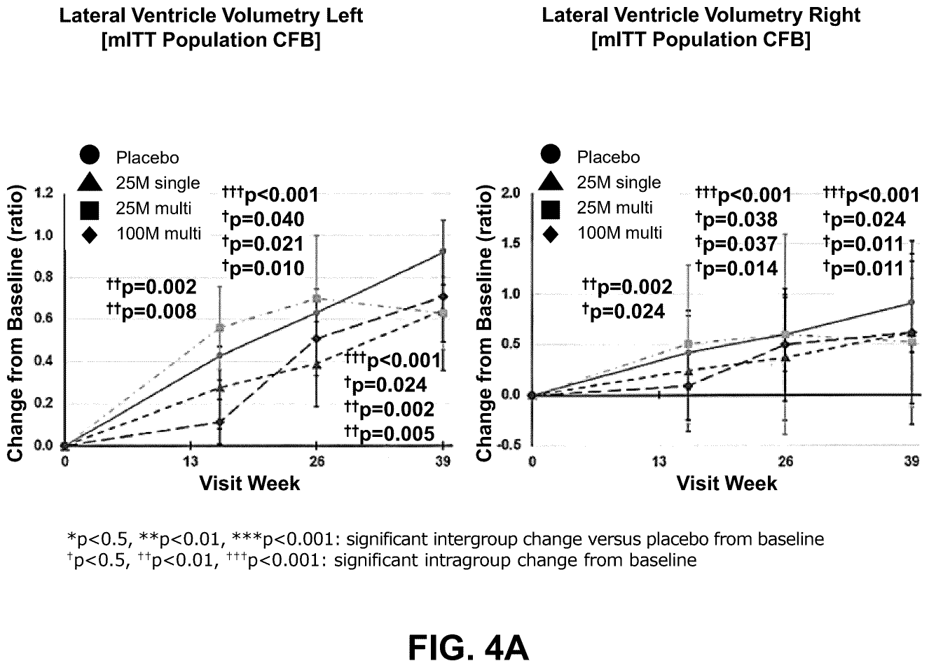

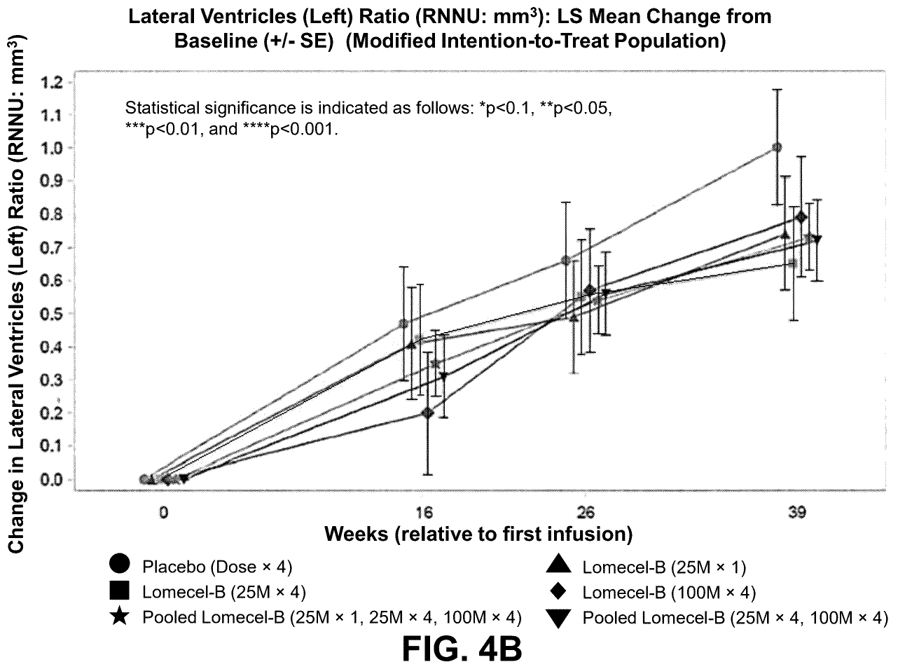

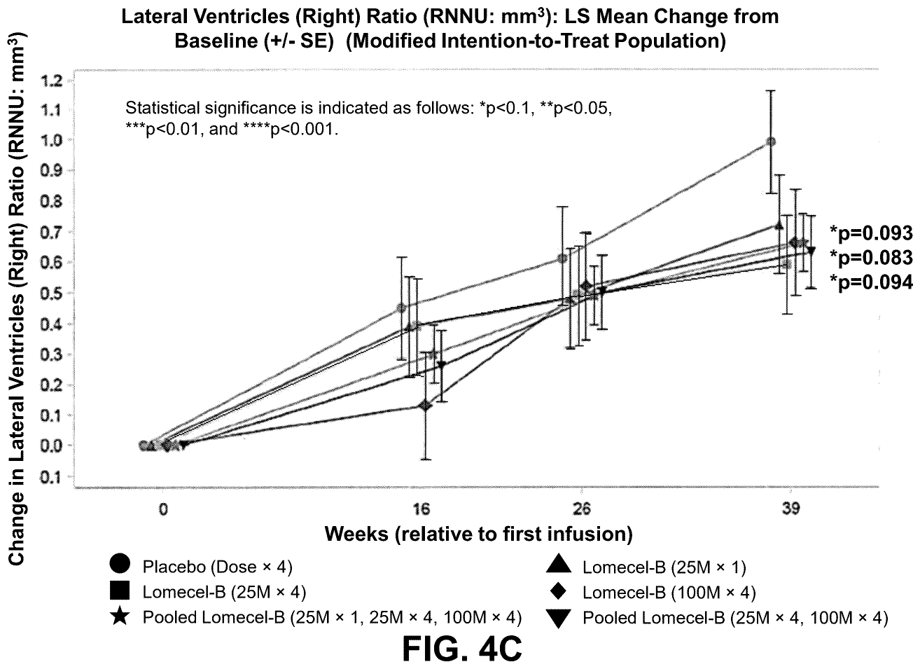

In some other cases, the volume measurement may indicate a suppressed increase in volume. In some cases, the suppressed increase in volume may pertain to a brain ventricle, including but not limited to a lateral ventricle (see FIGS. 4A-C), and/or the like, of the subject in need thereof after administration of the composition including the therapeutically effective amount of the allogenic MSCs. As nonlimiting examples, the suppressed increase in volume can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%.

In some embodiments, evaluating the one or more biomarkers may include measuring a concentration of one or more whole-blood biomarkers, one or more blood-plasma biomarkers, and one or more blood-serum biomarkers, among others.

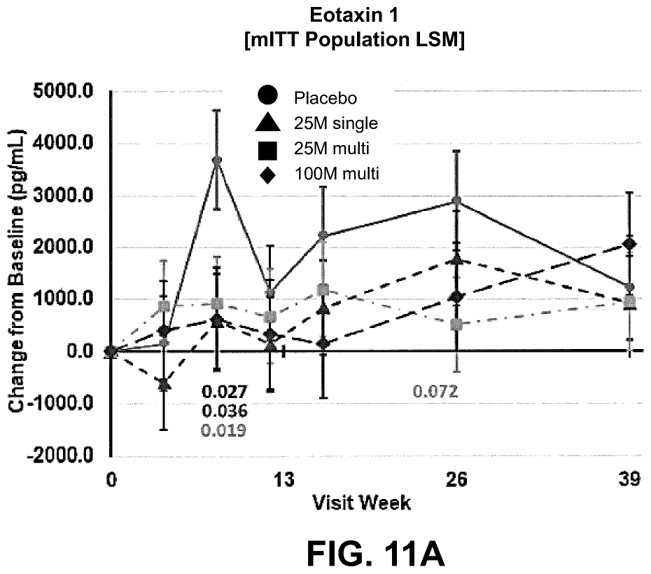

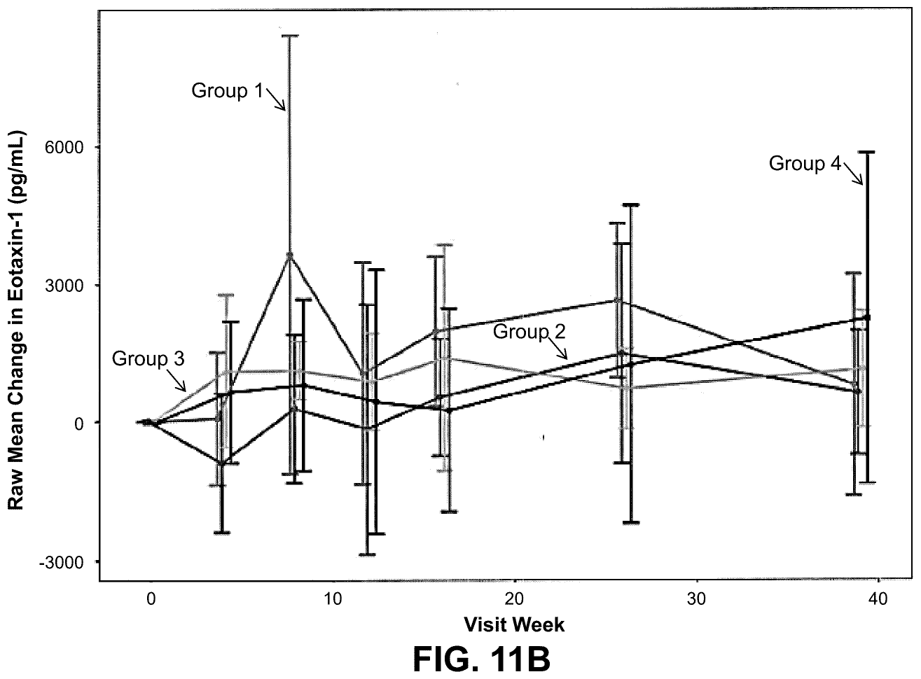

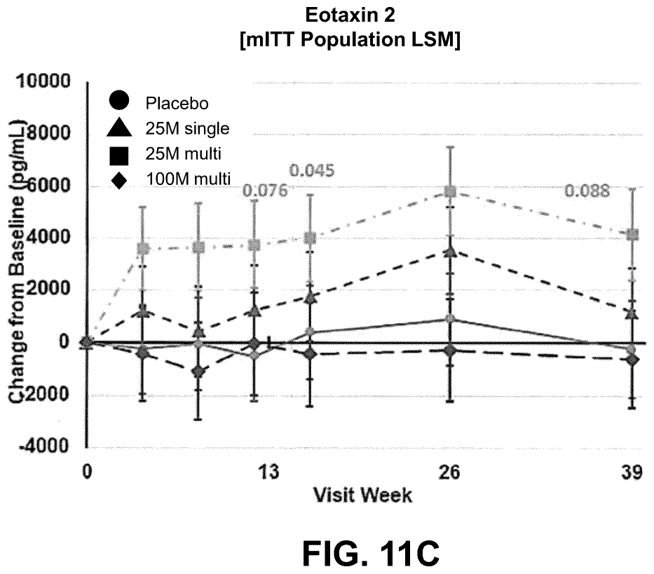

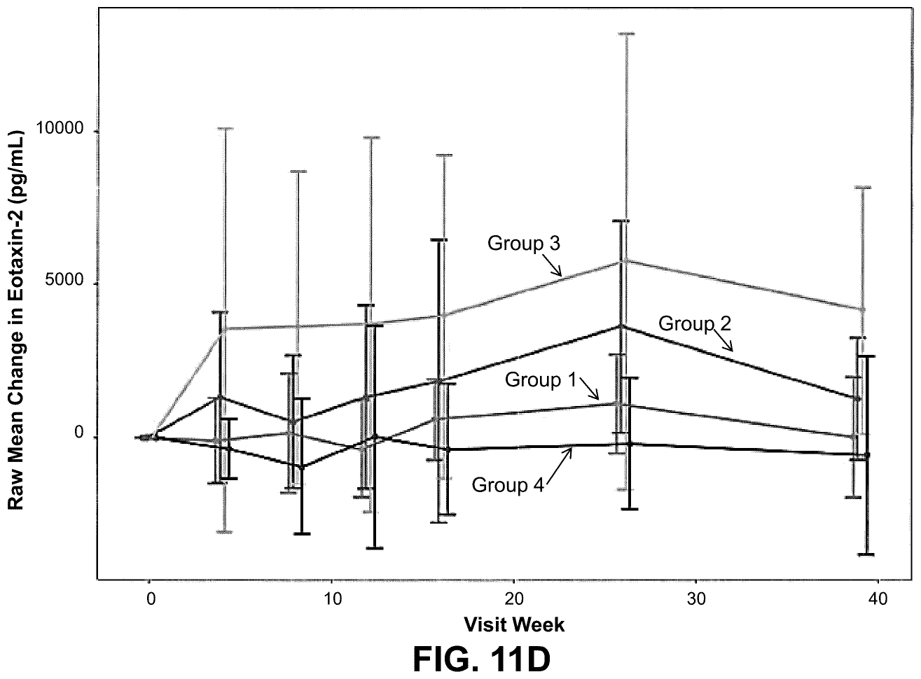

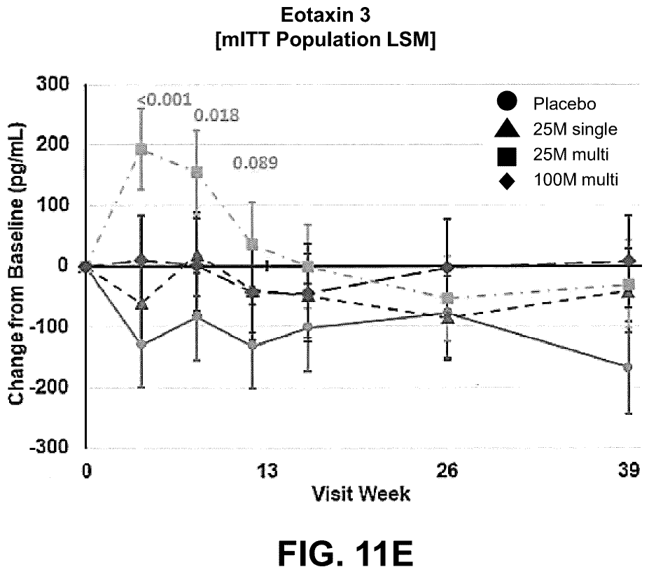

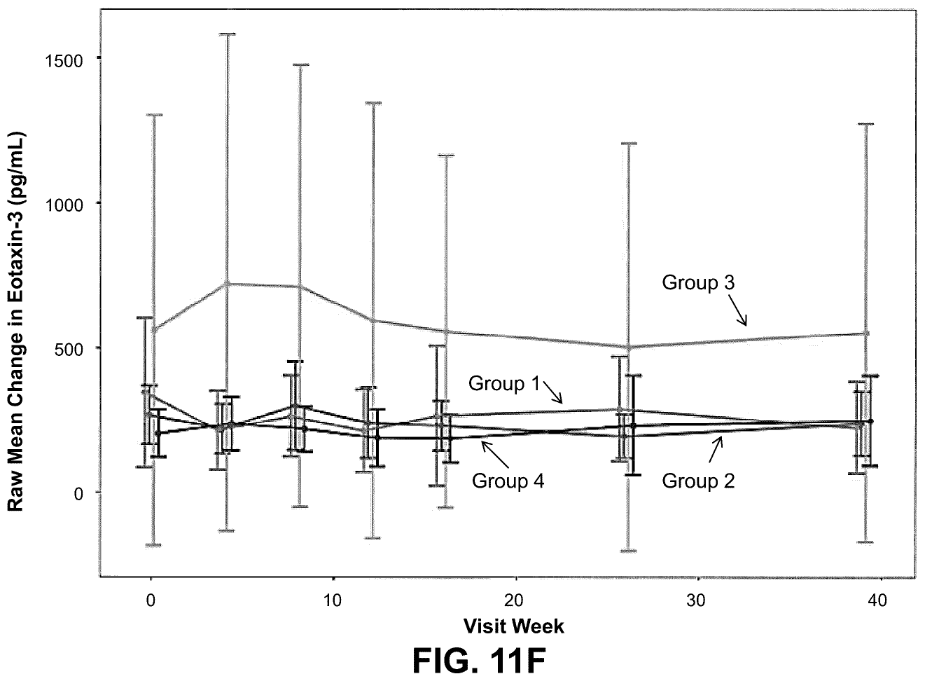

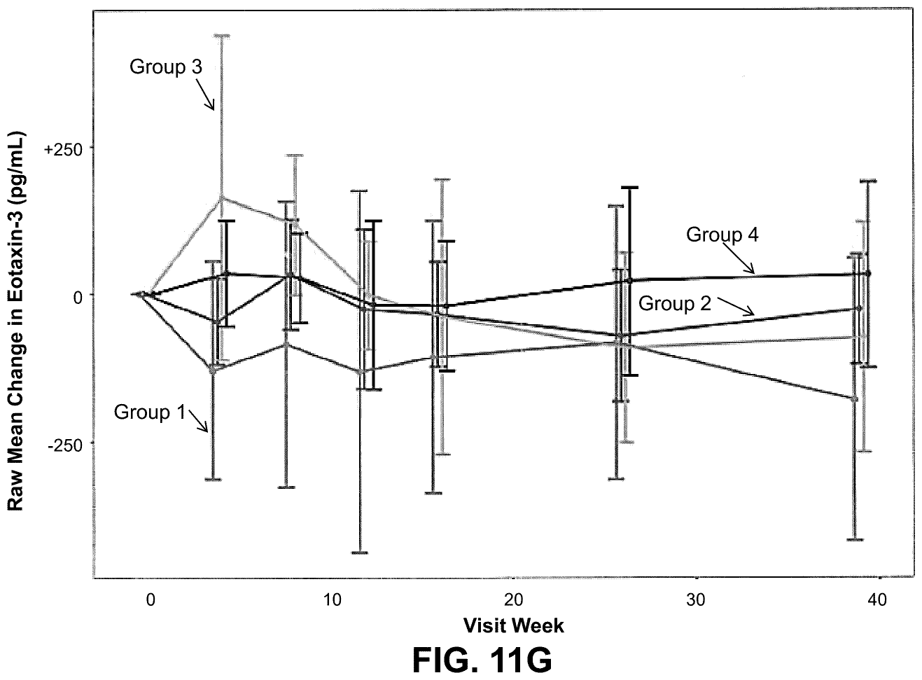

In some embodiments, evaluating the one or more biomarkers may include measuring a level or concentration of eotaxin 1 (FIGS. 11A, 11B), eotaxin 2 (FIGS. 11C, 11D), and/or eotaxin 3 (FIGS. 11E-G). As a nonlimiting example, Table 1 includes exemplary statistical data collected from Groups 1-4 pertaining to concentrations of eotaxin 3 (pg/mL). The lowest limit of quantification (LLOQ) was determined to be 28.2 pg/mL. 0% of baseline and follow-up values were below LLOQ.

TABLE 1
Statistical Data Pertaining to Eotaxin 3.

| Baseline | Group 1 | Group 2 | Group 3 | Group 4 |
|---|---|---|---|---|
| Count | 12 | 12 | 13 | 11 |
| Mean | 346.3667 | 268.85 | 560.6231 | 205.6909 |
| SD | 404.9575 | 159.5679 | 1230.367 | 122.5764 |
| Median | 202.15 | 222.65 | 173.5 | 175.3 |
| Min | 38.5 | 39.6 | 103.6 | 57.9 |
| Max | 1215 | 530.6 | 4634 | 461 |

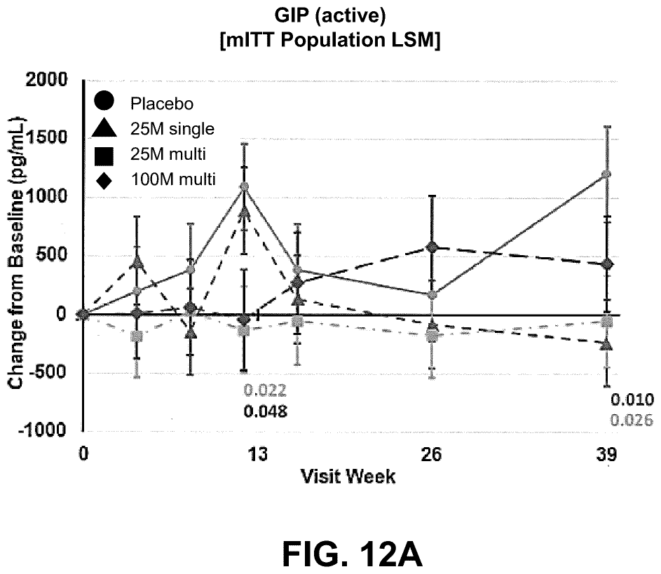

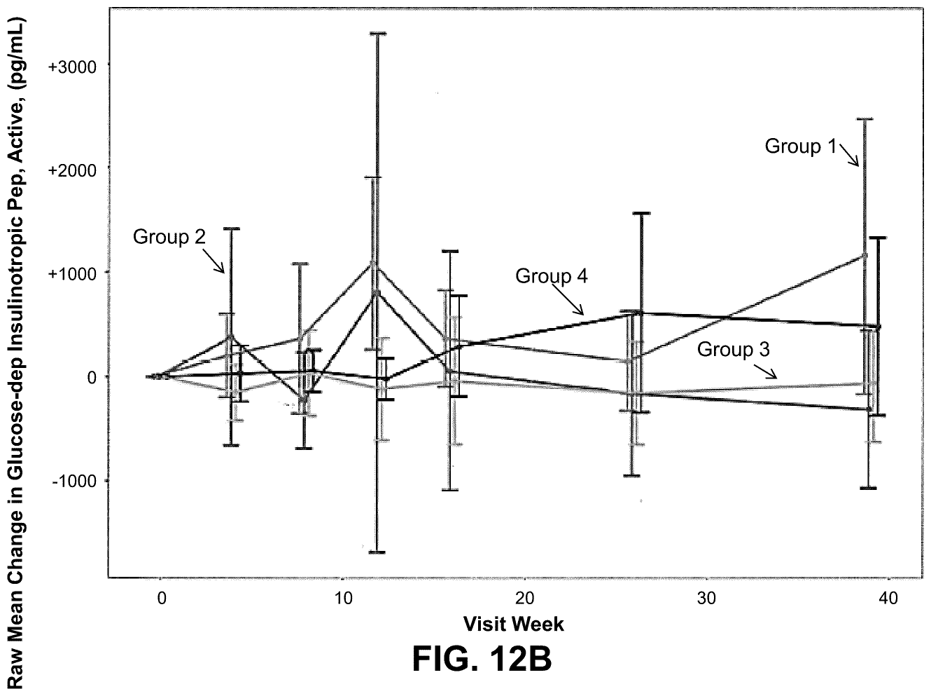

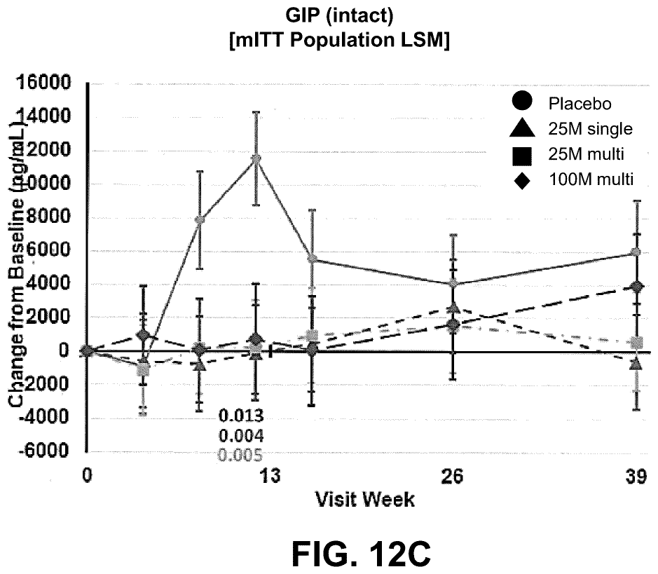

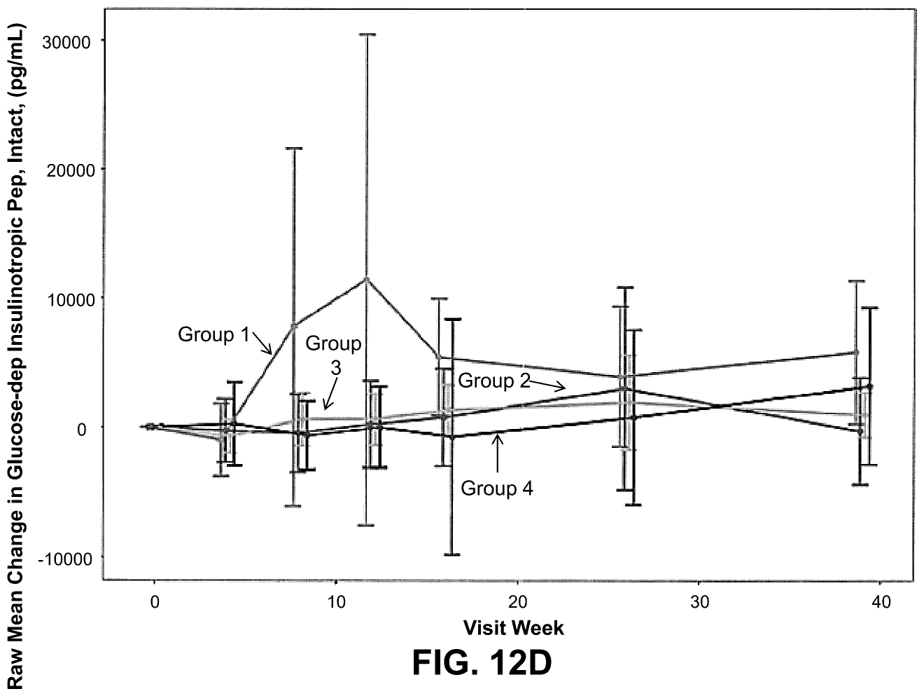

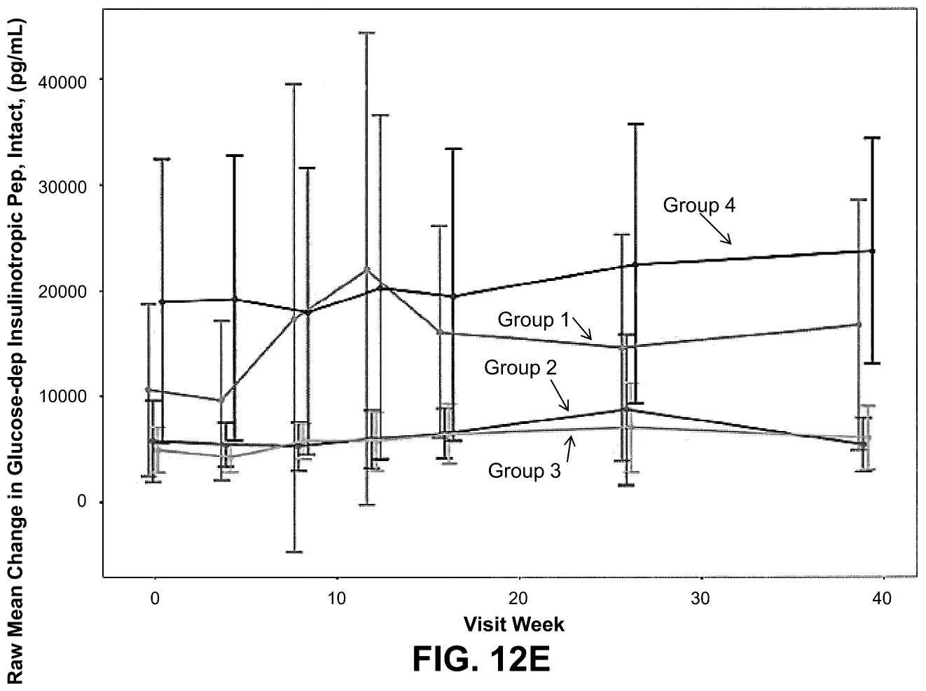

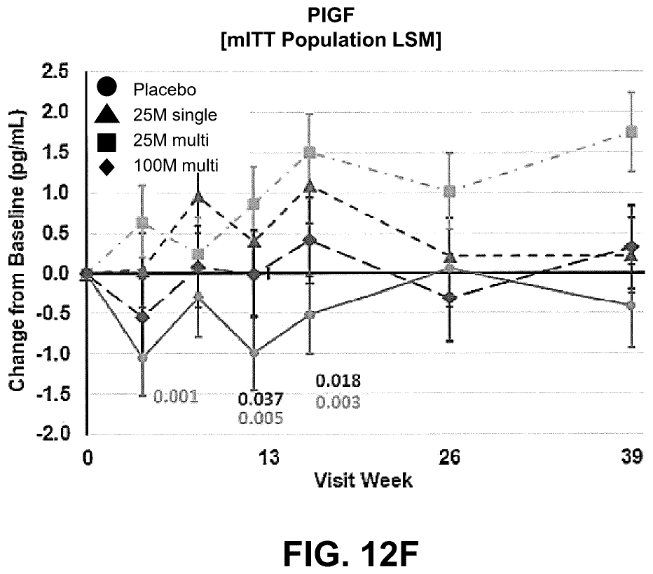

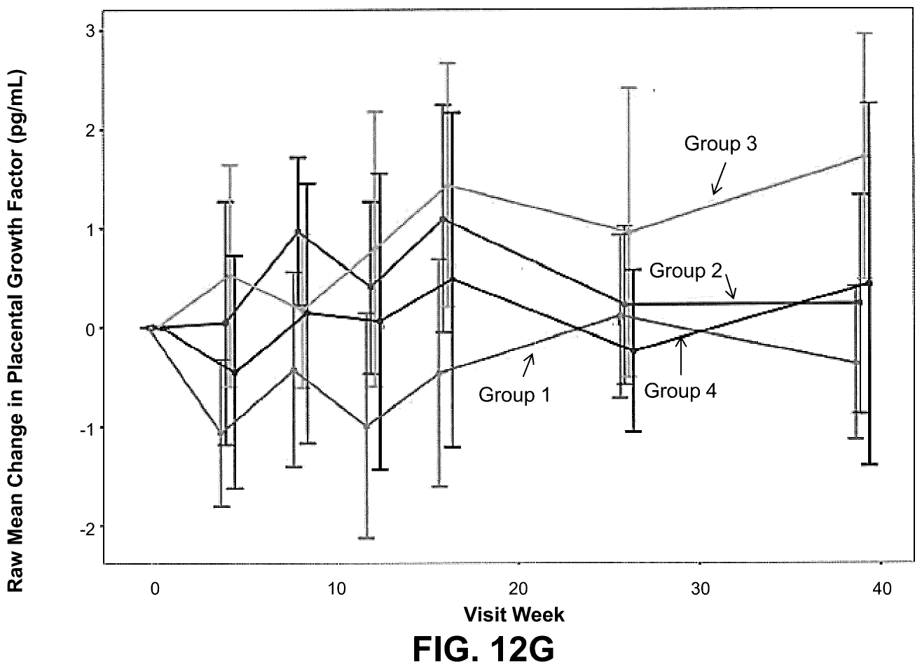

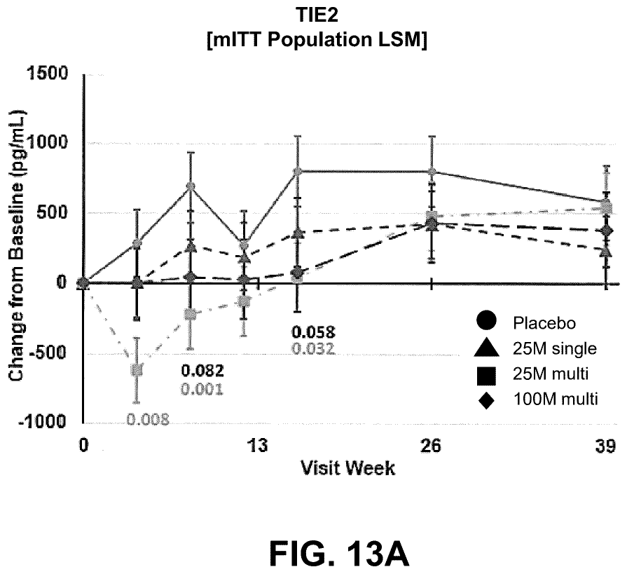

In some embodiments, evaluating the one or more biomarkers may include measuring a level or concentration of active glucose-dependent insulinotropic polypeptide (GIP, FIGS. 12A, 12B). In some embodiments, evaluating the one or more biomarkers may include measuring a level or concentration of intact GIP (FIGS. 12C-E). As a nonlimiting example, Table 2 includes exemplary statistical data collected from Groups 1-4 pertaining to concentrations of intact GIP (pg/mL). The LLOQ was determined to be 542.97 pg/mL. 0% of baseline and follow-up values were below LLOQ.

TABLE 2
Statistical Data Pertaining to Intact GIP.

| Baseline | Group 1 | Group 2 | Group 3 | Group 4 |
|---|---|---|---|---|
| Count | 12 | 12 | 13 | 11 |
| Mean | 25.71667 | 20.21667 | 26.62308 | 28.40909 |
| SD | 10.10588 | 9.214892 | 17.35752 | 11.54638 |
| Median | 26.3 | 19.95 | 19.4 | 30.1 |
| Min | 13.3 | 8.1 | 4.7 | 7.5 |
| Max | 43.9 | 40.2 | 61.2 | 47.5 |

In some embodiments, evaluating the one or more biomarkers may include measuring a level or concentration of placental growth factor (plGF, FIGS. 12F, 12G).

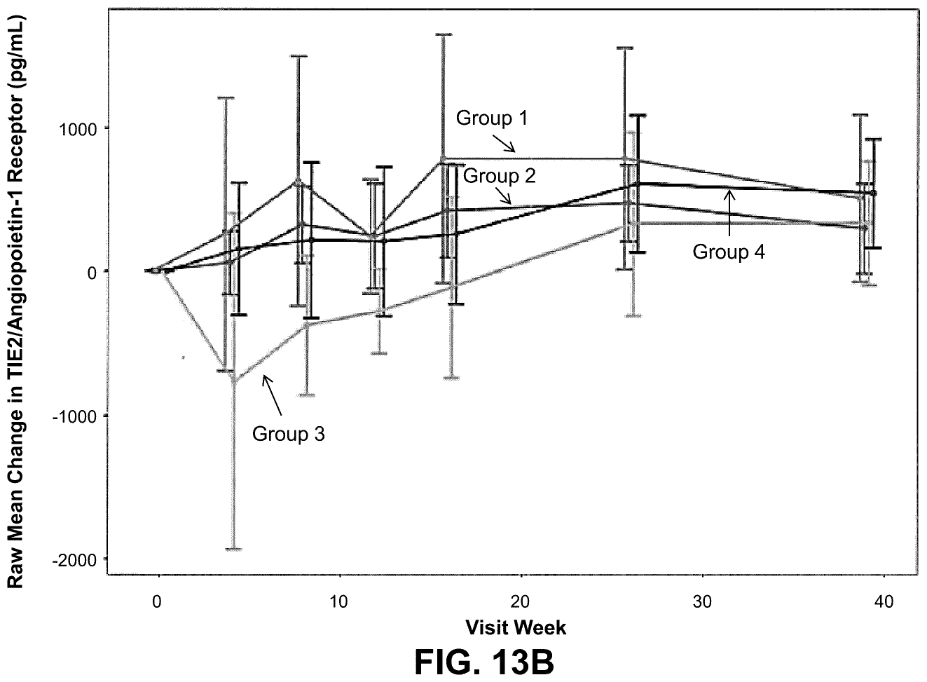

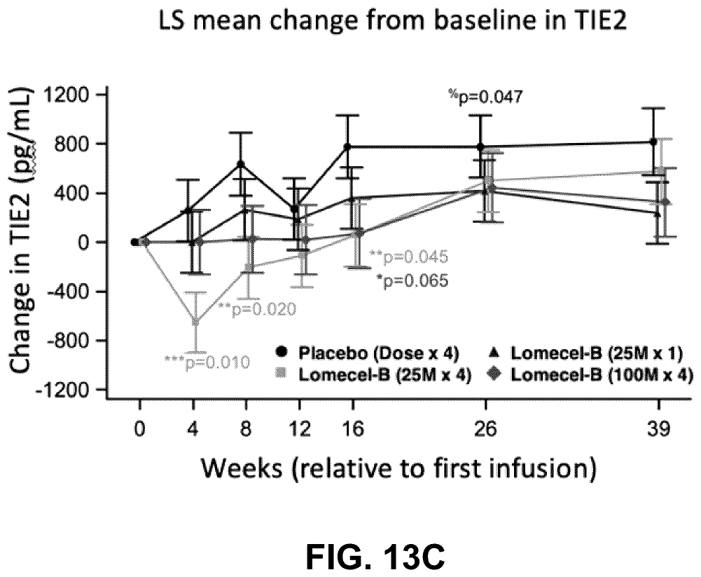

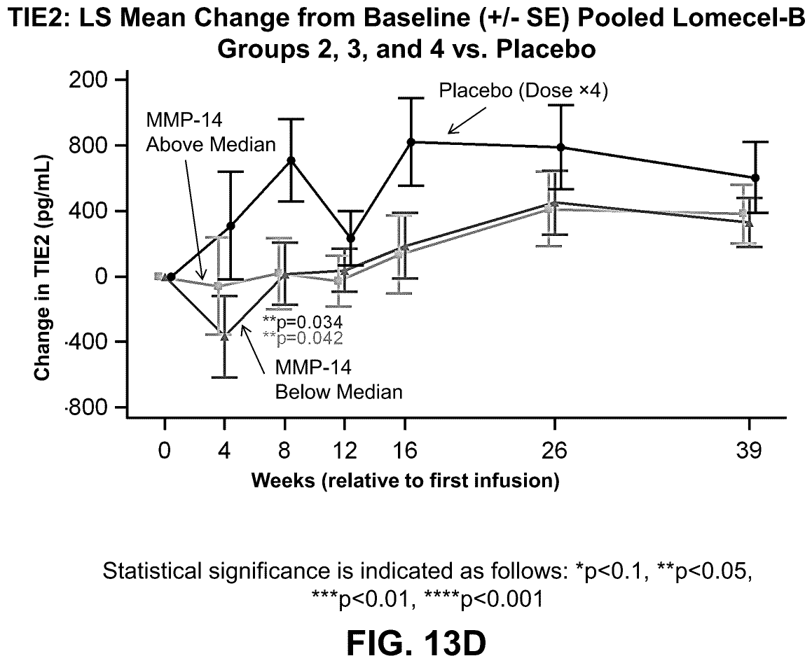

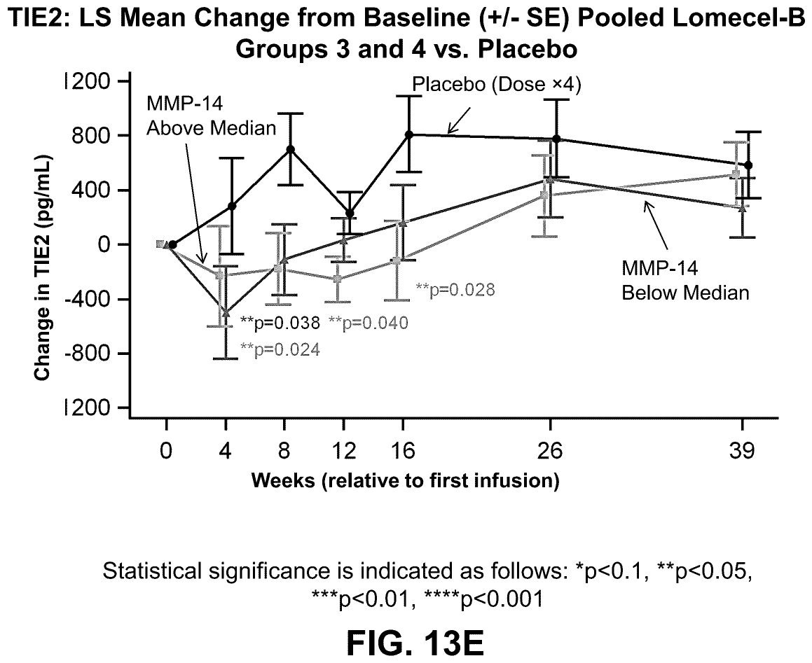

In some embodiments, evaluating the one or more biomarkers may include measuring a level or concentration of TIE2 including sTIE2 (FIGS. 13A-E). In some embodiments, one or more biomarkers may include or otherwise indicate an increase in the level of sTIE2 in the blood serum or plasma. In preferred embodiments, a reduction in the level of sTIE2 in the blood serum or plasma after the administration of the composition including allogeneic MSCs may be indicative of the efficacy of the treatment. Consistent with previous studies, a reduction in TIE2 levels observed in blood serum could reflect a decrease in the degradation and/or “shedding” of the extracellular portion of the TIE2 receptor after cleavage by metalloproteases, which are reported to be upregulated in AD (Idowu, T. O., et al.; Liao, M. C. & Van Nostrand, W. E. Biochemistry, 2010; 49:1127-1136; Yamada, T., et al., “White matter microglia produce membrane-type matrix metalloprotease, an activator of gelatinase A, in human brain tissues”, Acta Neuropathol., 1995; 90:421-424).

In some embodiments, evaluating the one or more biomarkers may include measuring a level or concentration of an angiopoietin-1 receptor.

In some embodiments, evaluating the one or more biomarkers may include measuring a level or concentration of MMP-14. Additional details will be provided below in this disclosure.

In some embodiments, evaluating the one or more biomarkers may include measuring a level or concentration of TIMP2. In some embodiments, evaluating the one or more biomarkers may include measuring a level or concentration of amyloid beta peptide with 40 amino acids (Aβ40), interleukin 1β (IL-1β), interleukin 2 (IL-2), interleukin 4 (IL-4), interleukin 6 (IL-6), interleukin 10 (IL-10), and/or interleukin 13 (IL-13), among others. Tables 3A and 3B summarize the findings based on various biomarkers with >25% below LLOQ at Week 16 and Week 39, respectively. All Biomarkers in Tables 3A-B have >25% of samples with values below LLOQ. LSM=Least Square Means; LSM Diff=LSM Difference versus Placebo. Data entries in bold represent statistically significant values (p<0.05).

TABLE 3A
Summary of Biomarkers with >25% Below LLOQ (LSM, Week 16)

| | | | Group 1 | Group 2 | | |
|---|---|---|---|---|---|---|
| | | | Placebo | Lomecel-B ™ 25M × 1 | | |
| | | % Below | LSM | LSM | LSM Diff | p- |
| Endpoint | Category | LLOQ | (SE) | (SE) | (SE) | value |
| Aβ38 | Amyloid | 73.19 | 23.01 | −25.20 | −48.20 | 0.950 |
| | | | (556.543) | (532.437) | (768.894) | |
| Aβ40 | Amyloid | 53.94 | −44.57 | −21.28 | 23.29 | 0.636 |
| | | | (35.595) | (34.056) | (49.179) | |
| Aβ42 | Amyloid | 47 | 13.87 | 12.05 | −1.82 | 0.979 |
| | | | (49.677) | (47.522) | (68.627) | |
| bFGF | Angiogenesis | 60.25 | 1.82 | −3.61 | −1.80 | 0.640 |
| | | | (2.772) | (2.641) | (3.830) | |
| IL-10 | Proinflammatory | 91.17 | −6.96 | −6.88 | 0.08 | 0.807 |
| | | | (0.226) | (0.216) | (0.312) | |
| IL-12p70 | Proinflammatory | 99.68 | No Data - All samples below LLOQ | | | |
| IL-13 | Proinflammatory | 98.74 | 0.33 | −0.34 | 0.00 | 0.991 |
| | | | (0.182) | (0.174) | (0.251) | |
| IL-17 | Metabolic | 38.17 | 22.97 | 14.11 | −8.86 | 0.550 |
| | | | (10.670) | (10.223) | (14.813) | |
| IL-1β | Proinflammatory | 93.38 | −37.54 | −37.56 | −0.02 | 0.989 |
| | | | (0.872) | (0.834) | (1.205) | |
| IL-2 | Proinflammatory | 99.37 | −0.10 | 0.10 | 0.00 | 0.987 |
| | | | (0.052) | (0.049) | (0.071) | |
| IL-4 | Proinflammatory | 99.05 | −0.56 | −0.56 | 0.00 | 0.993 |
| | | | (0.008) | (0.008) | (0.011) | |
| IL-6 | Proinflammatory | 58.68 | −72.27 | −72.23 | 0.04 | 0.998 |
| | | | (10.063) | (9.625) | (13.899) | |
| YKL-40 | Immuno-Oncology | 44.48 | −49639.16 | −7588.77 | 42050.39 | 0.289 |
| | | | (28564.840) | (27349.380) | (39542.340) | |

| | Group 3 | | | Group 4 | | |
|---|---|---|---|---|---|---|
| | Lomecel-B ™ 25M × 4 | | | Lomecel-B ™ 100M × 4 | | |
| | LSM | LSM Diff | p- | LSM | LSM Diff | p- |
| Endpoint | (SE) | (SE) | value | (SE) | (SE) | value |
| Aβ38 | 13.03 | −9.97 | 0.990 | 461.22 | −484.22 | 0.562 |
| | (531.996) | (769.427) | | (617.792) | (832.669) | |
| Aβ40 | −12.44 | 32.13 | 0.515 | −58.62 | −14.05 | 0.792 |
| | (34.025) | (49.213) | | (39.500) | (53.237) | |
| Aβ42 | 16.78 | 2.91 | 0.966 | −101.59 | −115.46 | 0.122 |
| | (47.483) | (68.675) | | (55.148) | (74.334) | |
| bFGF | −2,79 | −0.97 | 0.8000 | −6.59 | −4.77 | 0.249 |
| | (2.641) | (3.838) | | (3.048) | (4.124) | |
| IL-10 | 6.92 | 0.04 | 0.901 | 06.89 | 0.07 | 0.842 |
| | (0.217) | (0.314) | | (0.250) | (0.336) | |
| IL-12p70 | No Data - All samples below LLOQ | | | | | |
| IL-13 | −0.33 | 0.00 | 0.992 | 0.15 | 0.48 | 0.075 |
| | (0.174) | (0.253) | | (0.201) | (0.271) | |
| IL-17 | 12.74 | −10.23 | 0.489 | 31.30 | 8.32 | 0.600 |
| | (10.173) | (14.751) | | (11.780) | (15.842) | |
| IL-1β | −37.53 | 0.01 | 0.993 | −37.04 | 0.50 | 0.702 |
| | (0.836) | (1.211) | | (0.963) | (1.297) | |
| IL-2 | −0.10 | 0.00 | 0.999 | −0.10 | 0.00 | 0.982 |
| | (0.049) | (0.072) | | (0.057) | (0.077) | |
| IL-4 | −0.56 | 0.00 | 0.987 | −0.56 | 0.00 | 0.990 |
| | (0.008) | (0.011) | | (0.009) | (0.012) | |
| IL-6 | −72.02 | 0.25 | 0.986 | −66.12 | 6.15 | 0.682 |
| | (9.651) | (13.968) | | (11.105) | (14.968) | |
| YKL-40 | 8777.01 | 58416.17 | 0.141 | 1375.25 | 51015.41 | 0.232 |
| | (27293.274) | (39515.145) | | (31521.118) | (42509.079) | |

TABLE 3B
Summary of Biomarkers with >25% Below LLOQ (LSM, Week 39)

| | | | Group 1 | Group 2 | | |
|---|---|---|---|---|---|---|
| | | | Placebo | Lomecel-B ™ 25M × 1 | | |
| | | % Below | LSM | LSM | LSM Diff | p- |
| Endpoint | Category | LLOQ | (SE) | (SE) | (SE) | value |
| Aβ38 | Amyloid | 73.19 | 730.01 | −37.99 | −767.99 | 0.332 |
| | | | (584.613) | (532.437) | (789.047) | |
| Aβ40 | Amyloid | 53.94 | 59.50 | −50.14 | −109.63 | 0.031 |
| | | | (37.390) | (34.056) | (50.468) | |
| Aβ42 | Amyloid | 47 | 53.47 | 10.91 | −42.57 | 0.546 |
| | | | (52.182) | (47.522) | (70.426) | |
| bFGF | Angiogenesis | 60.25 | −5.96 | −4.51 | 1.44 | 0.714 |
| | | | (2.911) | (2.641) | (3.929) | |
| IL-10 | Proinflammatory | 91.17 | −6.99 | −6.98 | 0.01 | 0.981 |
| | | | (0.238) | (0.216) | (0.321) | |
| IL-12p70 | Proinflammatory | 99.68 | | No Data - All samples below LLOQ | | |
| IL-13 | Proinflammatory | 98.74 | −0.33 | −0.34 | −0.01 | 0.981 |
| | | | (0.191) | (0.174) | (0.258) | |
| IL-17 | Metabolic | 38.17 | 1.04 | 2.77 | 1.73 | 0.910 |
| | | | (11.222) | (10.223) | (15.223) | |
| IL-1β | Proinflammatory | 93.38 | −37.52 | −37.55 | −0.03 | 0.982 |
| | | | (0.916) | (0.834) | (1.236) | |
| IL-2 | Proinflammatory | 99.37 | −0.10 | −0.10 | 0.00 | 0.972 |
| | | | (0.054)) | (0.049) | (0.073) | |
| IL-4 | Proinflammatory | 99.05 | −0.56 | −0.56 | 0.00 | 0.984 |
| | | | (0.009) | (0.008) | (0.012) | |
| IL-6 | Proinflammatory | 58.68 | −72.25 | −72.53 | −0.28 | 0.984 |
| | | | (10.570) | (9.625) | (14.264) | |
| YKL-40 | Immuno-Oncology | 44.48 | −12443.18 | 03023.67 | 9419.51 | 0.817 |
| | | | (30014.190) | (27349.380) | (40595.850) | |

| | Group 3 | | | Group 4 | | |
|---|---|---|---|---|---|---|
| | Lomecel-B ™ 25M × 4 | | | Lomecel-B ™ 100M × 4 | | |
| | LSM | LSM Diff | p- | LSM | LSM Diff | p- |
| Endpoint | (SE) | (SE) | value | (SE) | (SE) | value |
| Aβ38 | 40.96 | −689.05 | 0.393 | −709.31 | −1439.32 | 0.084 |
| | (555.768) | (805.500) | | (585.667) | (829.148) | |
| Aβ40 | −14.32 | −73.82 | 0.153 | −21.62 | −81.12 | 0.127 |
| | (35.546) | (51.521) | | (37.441) | (53.008) | |
| Aβ42 | 19.21 | −34.26 | 0.634 | −75.12 | −128.59 | 0.084 |
| | (49.605) | (71.895) | | (52.277) | (74.015) | |
| bFGF | 0.93 | 6.89 | 0.088 | −5.39 | 0.57 | 0.891 |
| | (2.761) | (4.019) | | (2.891) | (4.105) | |
| IL-10 | 6.84 | 0.15 | 0.639 | −5.77 | 1.22 | <0.001 |
| | (0.227) | (0.329) | | (0.237) | (0.335) | |
| IL-12p70 | No Data - All samples below LLOQ | | | | | |
| IL-13 | 0.46 | 0.79 | 0.003 | −0.34 | −0.01 | 0.969 |
| | (0.182) | (0.264) | | (0.190) | (0.270) | |
| IL-17 | 1.11 | 0.07 | 0.997 | 11.54 | 10.50 | 0.507 |
| | (10.626) | (15.449) | | (11.168) | (15.785) | |
| IL-1β | −33.49 | 4.03 | 0.002 | −37.50 | 0.02 | 0.989 |
| | (0.874) | (1.268) | | (0.913) | (1.292) | |
| IL-2 | 0.16 | 0.25 | <0.001 | −0.10 | 0.00 | 0.955 |
| | (0.052) | (0.075) | | (0.054) | (0.076) | |
| IL-4 | −0.52 | 0.04 | 0.003 | −0.56 | 0.00 | 0.974 |
| | (0.008) | (0.012) | | (0.009) | (0.012) | |
| IL-6 | −22.70 | 49.55 | <0.001 | −70.46 | 1.79 | 0.905 |
| | (10.085) | (14.629) | | (10.533) | (14.913) | |
| YKL-40 | −184.68 | 12258.50 | 0.767 | 7703.94 | 20147.11 | 0.635 |
| | (28515.416) | (41387.047) | | (29896.386) | (42363.982) | |

### Potency Assays for Evaluating the Therapeutic Effects of MSCs

The invention described herein is also directed towards methods for evaluating potency of allogeneic MSCs using a potency assay. As used herein, a “potency assay” is a quantitative measure of biological activity. Ideally, a potency assay measures the ability of a product to elicit a specific response in a disease-relevant system. The activity measured in a potency assay represents an intended biological effect (e.g., mechanism of action) and is often related to a clinical response. Details described herein may be consistent with any detail disclosed in U.S. patent application Ser. No. 17/996,529 (attorney docket number 0085548-000151), filed on Apr. 20, 2021, entitled “POTENCY ASSAY”, the entirety of which is incorporated herein by reference.

In some embodiments, the potency assay may include an MMP-14 inhibition assay. Accordingly, in some embodiments, one or more biomarkers for determining the potency of MSCs to treat AD or HLHS in a subject in need thereof may include MMP-14. In preferred embodiments, the inhibition of MMP-14 may be directly related to the ability of MSCs to treat AD or HLHS. The inhibition of MMP-14 may be at any inhibition level deemed relevant and/or reasonable by a person of ordinary skill in the art, upon reviewing the entirety of this disclosure. As nonlimiting examples, the inhibition of MMP-14 (MMP-14i) can be from 0% to 10%, from 0.5% to 10%, from 1.0% to 10%, from 3% to 10%, from 5% to 10%, from 7% to 10%, from greater than 0% to less than or equal to 10%, from 10% to 50%, from 20% to 50%, from 30% to 50%, or greater than 50%. Additionally, and/or alternatively, inhibition level of MMP-14 may be expressed using percentage of inhibition per cell per liter (%/cell/L). As further nonlimiting examples, the inhibition of MMP-14 can be from 0 to 1×10⁻⁷%/cell/L, from 1×10⁻⁷%/cell/L to 2×10⁻⁷%/cell/L, from 2×10⁻⁷%/cell/L to 3×10⁻⁷%/cell/L, from 3×10⁻⁷%/cell/L to 4×10⁻⁷%/cell/L, from 4×10⁻⁷%/cell/L to 5×10⁻⁷%/cell/L, from 5×10⁻⁷%/cell/L to 6×10⁻⁷%/cell/L, from 6×10⁻⁷%/cell/L to 7×10⁻⁷%/cell/L, from 7×10⁻⁷%/cell/L to 8×10⁻⁷%/cell/L, from 8×10⁻⁷%/cell/L to 9×10⁻⁷%/cell/L, from 9×10⁻⁷%/cell/L to 1×10⁶%/cell/L, and/or the like. MMP-14i activity, when normalized to cell density, shows statistical significance in a responder analysis for the CADS score, MoCA, ADCS-ADL, and serum sTIE2 levels, consistent with details described above in this disclosure.

In some embodiments, the potency assay may include an enzyme-linked immunosorbent assay (ELISA). As used herein, an “enzyme-linked immunosorbent assay” or “ELISA” is a widely used technique in immunology to detect and quantify specific antigens or antibodies in a sample. ELISA involves immobilizing a target molecule (e.g., an antigen) on a surface, adding a sample that potentially contains an analyte (e.g., an antibody), adding an enzyme-labeled ligand (e.g., an enzyme-labelled antibody) that competes with the analyte in binding with the immobilized target molecule, and then adding a substrate that reacts with the enzyme to produce a detectable signal, usually a color change. ELISA is commonly used in diagnostics including disease detection, vaccine development, and measuring immune responses. A person of ordinary skill in the art, upon reviewing the entirety of this disclosure, will be able to recognize how ELISA may be applied to the invention described herein.

TIMP2 is highly expressed by Lomecel-B™. Accordingly, in some embodiments, one or more biomarkers for determining the potency of MSCs to treat AD or HLHS in a subject in need thereof may include TIMP2. In preferred embodiments, the expression level of TIMP2 may be directly related to the ability of MSCs to treat AD. The level of TIMP2 may be quantified using a TIMP2 ELISA, consistent with details described above. The expression level of TIMP2 may be any expression level deemed relevant and/or reasonable by a person of ordinary skill in the art, upon reviewing the entirety of this disclosure. As nonlimiting examples, the expression level of TIMP2 can be from 0 to 1×10⁻⁷ ng/cell/L, from 1×10⁻⁷ ng/cell/L to 2×10⁻⁷ ng/cell/L, from 2×10⁻⁷ ng/cell/L to 3×10⁻⁷ ng/cell/L, from 3×10⁻⁷ ng/cell/L to 4×10⁻⁷ ng/cell/L, from 4×10⁻⁷ ng/cell/L to 5×10⁻⁷ ng/cell/L, from 5×10⁻⁷ ng/cell/L to 6×10⁻⁷ ng/cell/L, from 6×10⁻⁷ ng/cell/L to 7×10⁻⁷ ng/cell/L, from 7×10⁻⁷ ng/cell/L to 8×10⁻⁷ ng/cell/L, from 8×10⁻⁷ ng/cell/L to 9×10⁻⁷ ng/cell/L, from 9×10⁻⁷ ng/cell/L to 1×10⁶ ng/cell/L, and/or the like.

In some embodiments, the potency assay may include a vascular endothelial growth factor A (VEGF-A) or a meso scale discovery (MSD) assay. Accordingly, in some embodiments, one or more biomarkers for determining the potency of MSCs to treat AD or HLHS in a subject in need thereof may include VEGF-A. In preferred embodiments, the expression level of VEGF-A may be directly related to the ability of MSCs to treat AD. The expression level of VEGF-A may be any expression level deemed relevant and/or reasonable by a person of ordinary skill in the art, upon reviewing the entirety of this disclosure. As nonlimiting examples, the expression level of VEGF-A can be from 0 to 1×10⁵ ng/cell/L, from 1×10⁻⁵ ng/cell/L to 2×10⁵ ng/cell/L, from 2×10⁵ ng/cell/L to 3×10⁻⁵ ng/cell/L, from 3×10⁻⁵ ng/cell/L to 4×10⁵ ng/cell/L, from 4×10⁵ ng/cell/L to 5×10⁵ ng/cell/L, from 5×10⁻⁵ ng/cell/L to 6×10⁻⁵ ng/cell/L, from 6×10⁻⁵ ng/cell/L to 7×10⁵ ng/cell/L, from 7×10⁻⁵ ng/cell/L to 8×10⁵ ng/cell/L, from 8×10⁻⁵ ng/cell/L to 9×10⁻⁵ ng/cell/L, from 9×10⁻⁵ ng/cell/L to 1×10⁴ ng/cell/L, and/or the like.

In some embodiments, the potency assay may include a matrix potency assay. In some embodiments, the potency assay may include two or more members selected from a group consisting of an MMP-14 inhibition assay, a TIMP2 ELISA, and a VEGF-A MSD assay. Alternatively, each individual assay could be used as a potency assay by itself.

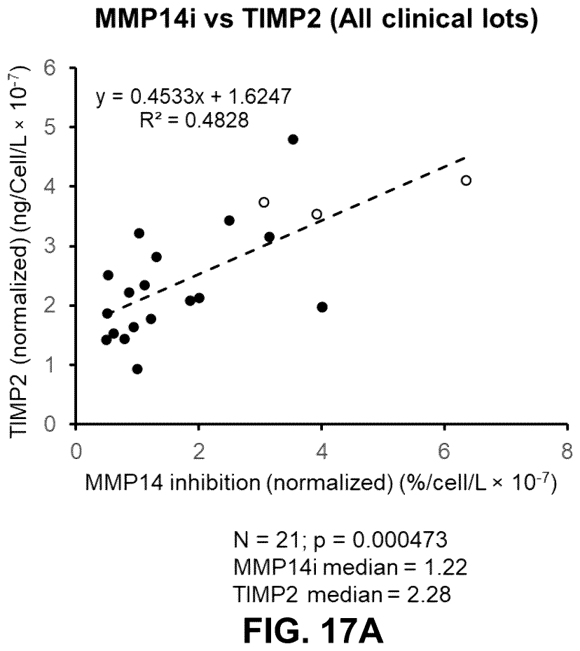

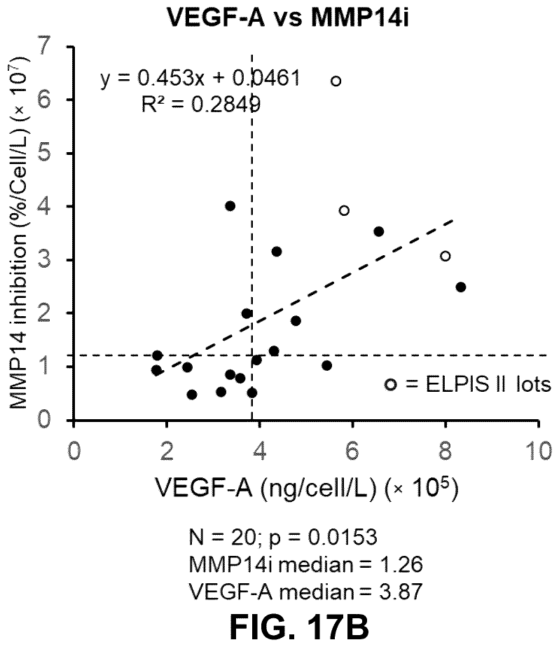

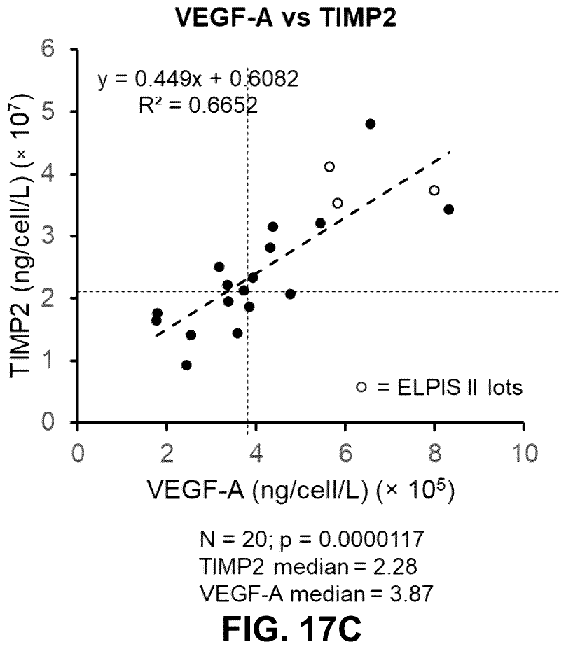

MMP-14i, level of TIMP2, and level of VEGF-A may represent a trio of related potency factors. A matrix of two or all of these assays could represent a superior potency bioassay predictive of the efficacy of treatment among subjects. In the exemplary study described above, results from each pair of potency assay candidates showed a satisfactorily linear correlation with one another (FIGS. 14B, 17A-C), In particular, the MMP-14 inhibition assay and its counterpart, TIMP2 ELISA, may represent potency assay components that accurately predict responses among subjects (FIGS. 14B, 17A). It is worth noting that normalization to cell density enhances the level of correlation between MMP-14i and TIMP2 compared to raw data.

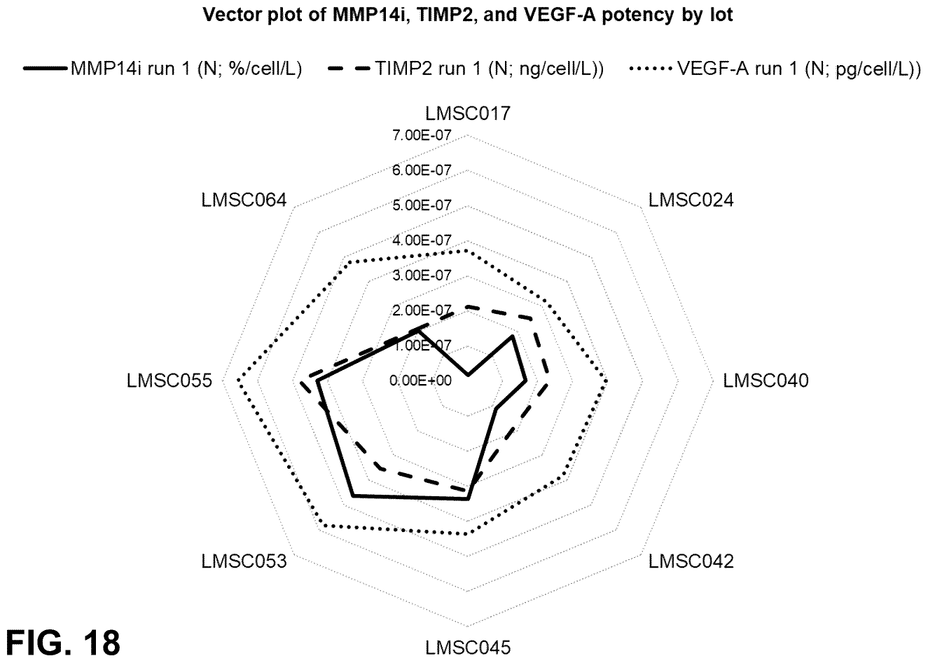

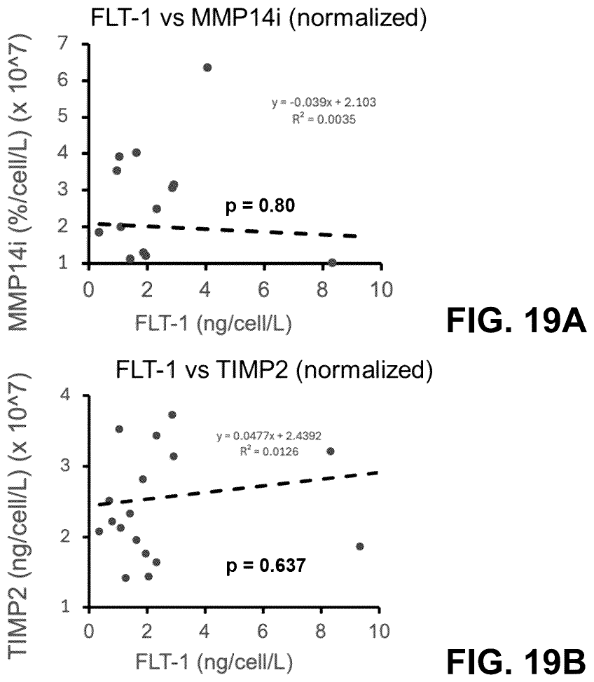

Additionally, the three potency assay candidates together showed a satisfactory correlation across lots of Lomecel-B™ (FIG. 18). It is worth noting that fms-related receptor tyrosine kinase 1 (FLT-1, also known as vascular endothelial growth factor receptor-1) did not show a linear correlation with levels of MMP-14i or TIMP2 (FIGS. 19A-B). This result potentially indicates certain specificity of potency factors.

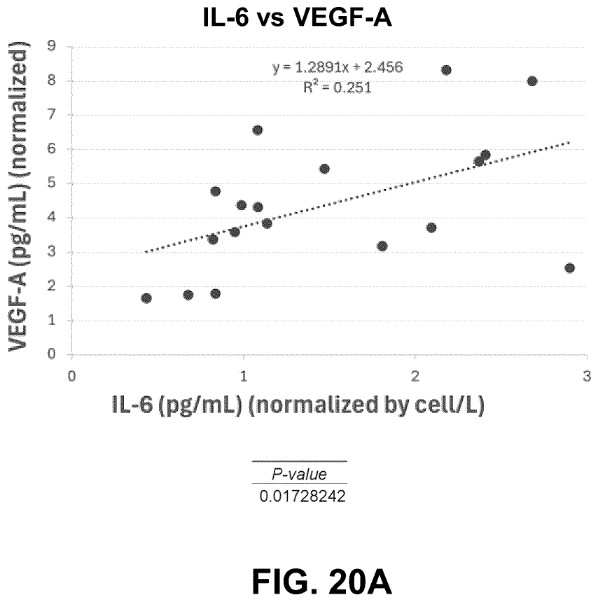

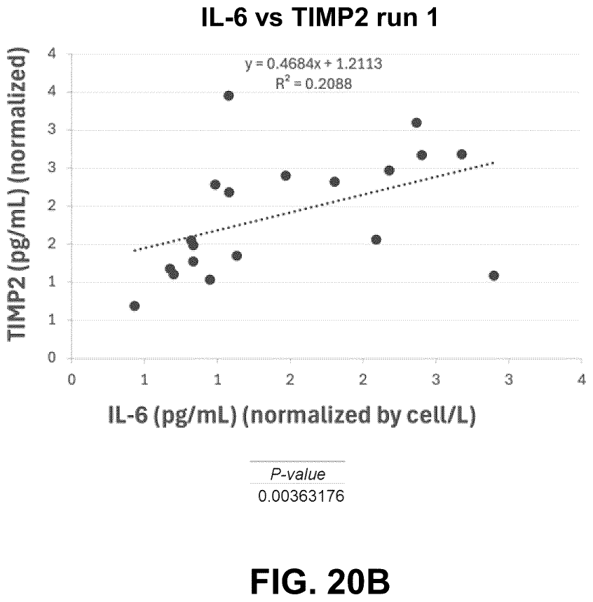

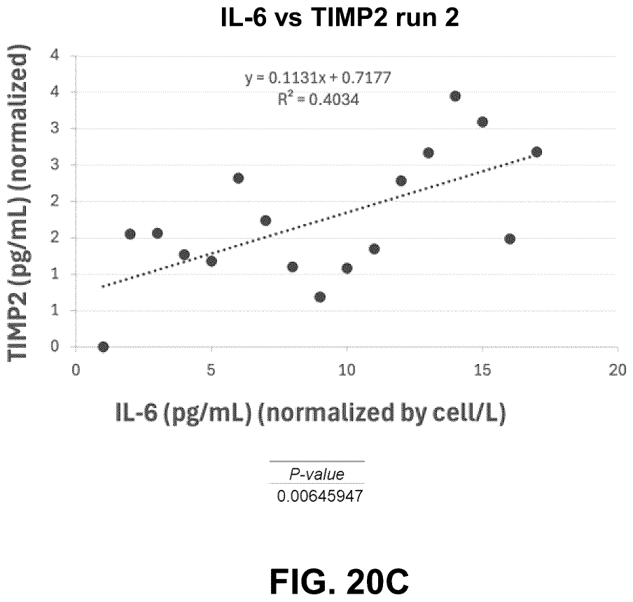

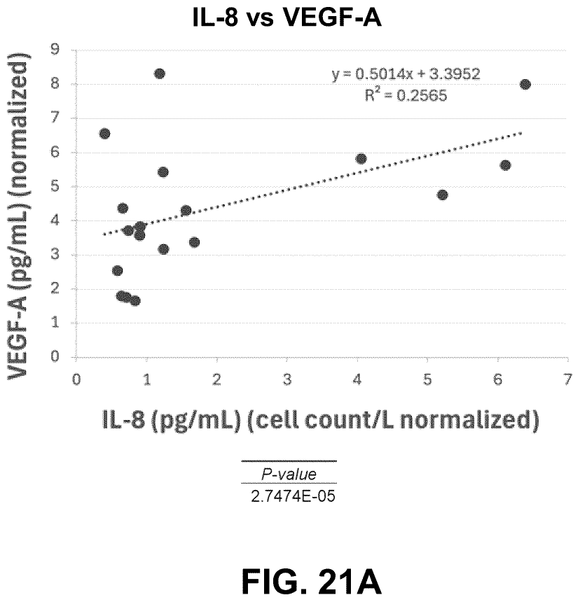

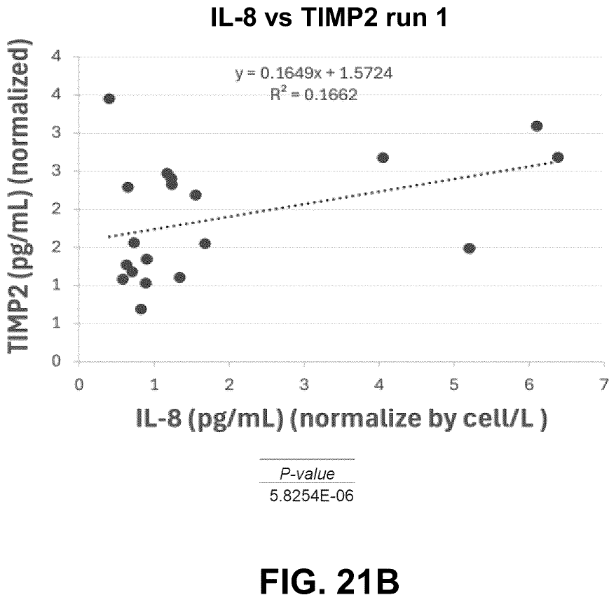

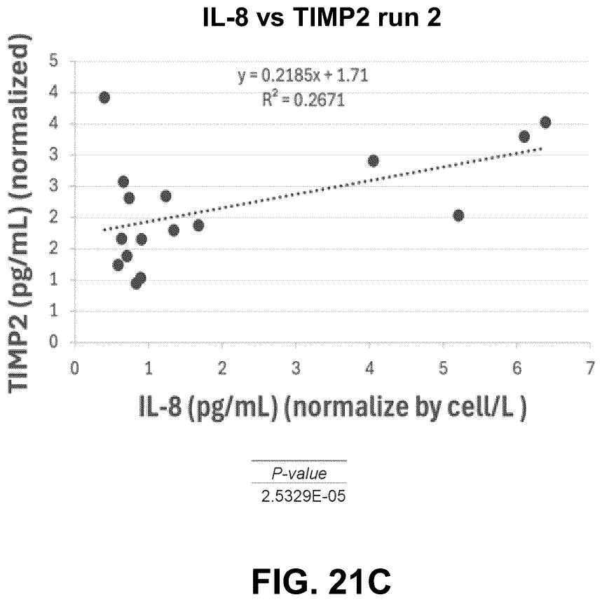

In some embodiments, the method for evaluating potency of allogeneic MSCs may further include measuring a concentration or expression level of IL-6, interleukin 8 (IL-8), and/or the like. In the same exemplary study described above, protein expression of IL-6 in Lomecel-B™ supernatants is significantly correlated with the expression levels of VEGF-A and TIMP2 (FIGS. 20A-C), and protein expression of IL-8 in Lomecel-B™ supernatants is also significantly correlated with the expression levels of VEGF-A and TIMP2 (FIGS. 21A-C). Similarly, IL-6 and IL-8 expressions also strongly correlate with levels of MMP-14i.

Experimental details for preparation of potency assays are provided below in this disclosure.

### Lomecel-B™ Supernatant

Lomecel-B™ supernatant can be collected at the time of harvest, or at any pre-freeze timepoint. Sample collection can also occur after thawing and culturing the cells to any collection timepoint. In the exemplary data below, results are shown from the harvest timepoint. Supernatant samples were frozen and stored at −80° C. until use.

### MMP-14 Inhibition Assay

The ability of Lomecel-B™ conditioned media to inhibit MMP-14 activity was assessed using the fluorometric MMP-14 Inhibitor Screening Kit (ab284518) from Abcam. Briefly, kit reagents were thawed to room temperature and prepared according to the manufacturer's instructions. Immediately before use, MMP-14 enzyme solution was diluted 1:50 with MMP-14 assay buffer, and MMP-14 inhibitor control was diluted 1:20 with the MMP-14 assay buffer. The following were added to a transparent-bottom black-walled 96-well plate (Corning) in duplicate:

Inhibitor control (IC) received 10 μL of diluted MMP-14 inhibitor control. Enzyme control (EC) received 10 μL of MMP-14 assay buffer. Media control (MC) received 10 μL of unconditioned Lomecel-B™ media containing 20% FBS (Corning). 10 μL of Lomecel-B™-conditioned media was added to respective sample wells. 50 μL of MMP-14 enzyme was added to all controls and samples, except for background control (BC) which only received 50 μL MMP-14 assay buffer and 10 μL of double-distilled water (ddH₂O). The 96-well plate was loaded onto a plate mixer at 450 rpm for 3 min, at 25° C., while shielded from light. Following mixing, the plate was incubated at room temperature (RT) for 10 min. Following incubation, substrate solution was added to all wells, and mixed at 450 rpm for 3 min, at 25° C., while shielded from light. Following incubation, fluorescence (Eₓ/Eₘ=325/420 nm) was kinetically measured at 3 min intervals for 1 hr at 37° C. using a Spectramax iD3 spectrophotometer (Molecular Devices). The slope was calculated for all controls and samples by dividing the net change in relative fluorescence units, ΔRFU (RFU₂−RFU₁), by the corresponding timespan, ΔT (i.e., T₂−T₁), with T₁ and T₂ being in the linear range. The ability of Lomecel-B™-conditioned medium to inhibit MMP-14 was determined using the following formula:

$$\% Relative inhibition = \frac{Slope of MC - Slope of Lomecel - B}{Slope of MC} \times 100 \%$$

A decline in slope represents inhibition.

### TIMP2 ELISA

TIMP2 protein was directly measured in Lomecel-B™ supernatant samples using an ELISA kit by Abcam (ab100653). Reagent preparation and procedures were performed according to the manufacturer's instructions. Media controls were included along with supernatant samples and measured in two technical replicates deposited in transparent-bottom, black-walled 96-well plates. Colorimetric detection was performed using a Spectramax iD3 spectrophotometer (Molecular Devices) reading at 450 nm. The seven standard curve points were plotted in log-log fashion. A linear model applied to fit the data, with R² values >0.99. Titration of supernatant showed an ideal dilution of 1:100. Initial TIMP2 values in Lomecel-B™ supernatant (two separate lots) were in the range of 10-30 ng/mL.

Exemplary experimental results are summarized below in Table 4.

TABLE 4
A Summary of Test Results for a List of Potency Assay Analytes.

| | % De- | | | | Number |
|---|---|---|---|---|---|
| | tectable | | | | of |
| | (above | | | Range | subjects |
| Analyte | LLOQ) | Mean | Median | (min to max) | (n) |
| MMP-14i | 100 | 1.92 × 10⁻⁴ | 1.26 × 10⁻⁴ | 5.86 × 10⁻⁴ | 22 |
| | | (%/cell) | (%/cell) | (%/cell) | |
| TIMP2 | 100 | 2.85 × 10⁻⁴ | 2.53 × 10⁻⁴ | 8.77 × 10⁻⁴ | 22 |
| | | (ng/cell) | (ng/cell) | (ng/cell) | |
| VEGF-A | 100 | 4.33 × 10⁻² | 3.88 × 10⁻² | 6.55 × 10⁻² | 20 |
| | | (pg/cell) | (pg/cell) | (pg/cell) | |
| VEGF-C | 100 | 1.35 × 10⁻³ | 1.16 × 10⁻³ | 1.52 × 10⁻³ | 20 |
| | | (pg/cell) | (pg/cell) | (pg/cell) | |
| VEGF-D | 20 | 1.95 × 10⁻⁴ | 1.76 × 10⁻⁴ | 1.19 × 10⁻⁴ | 20 |
| | | (pg/cell) | (pg/cell) | (pg/cell) | |
| FGF-2 | 15 | 9.47 × 10⁻³ | 8.88 × 10⁻³ | 7.93 × 10⁻³ | 20 |
| | | (pg/cell) | (pg/cell) | (pg/cell) | |
| PIGF | 100 | 5.69 × 10⁻⁴ | 4.83 × 10⁻⁴ | 1.31 × 10⁻³ | 20 |
| | | (pg/cell) | (pg/cell) | (pg/cell) | |
| FLT-1 | 100 | 2.41 × 10⁻⁴ | 1.75 × 10⁻⁴ | 9.0 × 10⁻⁴ | 20 |
| | | (pg/cell) | (pg/cell) | (pg/cell) | |
| IL-2 | 0 | ND | ND | ND | 25 |
| IL-4 | 0 | ND | ND | ND | 25 |
| IL-6 | 100 | 1.81 × 10⁻² | 1.52 × 10⁻² | 3.56 × 10⁻² | 25 |
| | | (pg/cell) | (pg/cell) | (pg/cell) | |
| IL-8 | 100 | 1.06 × 10⁻³ | 8.84 × 10⁻⁴ | 3.73 × 10⁻³ | 25 |
| | | (pg/cell) | (pg/cell) | (pg/cell) | |
| IL-10 | 0 | ND | ND | ND | 25 |
| IL-12 | 0 | ND | ND | ND | 25 |
| p70 | 0 | ND | ND | ND | 25 |
| IL-13 | 0 | ND | ND | ND | 25 |
| IL-1b | 0 | ND | ND | ND | 25 |
| TNF-a | 0 | ND | ND | ND | 25 |
| INF-g | 0 | ND | ND | ND | 25 |

### Impact of Freeze-Thaw Cycles

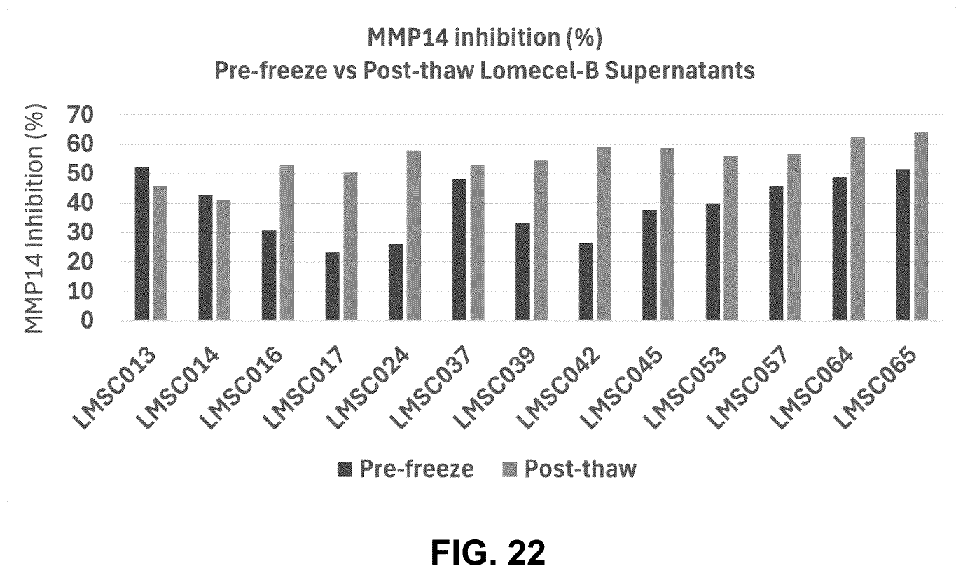

Referring now to FIG. 22, FIG. 22 depicts exemplary data comparing the MMP-14 inhibition potency between pre-freeze vs post-thaw Lomecel-B™ supernatants across 26 lots of Lomecel-B cells. Lomecel-B™ supernatants showed higher MMP-14 inhibition activity in all 26 tested lots (13 pre-freeze lots and 13 post-thaw lots). Freeze-thaw cycles therefore do not have a significant negative impact on the MMP14 inhibition activity of Lomecel-B™ cells. Instead, the post-thaw supernatant showed an elevated MMP14 inhibition activity compared to the pre-freeze supernatant. These results directly and positively address the impact of cell freeze-thaw cycles on cell potency.

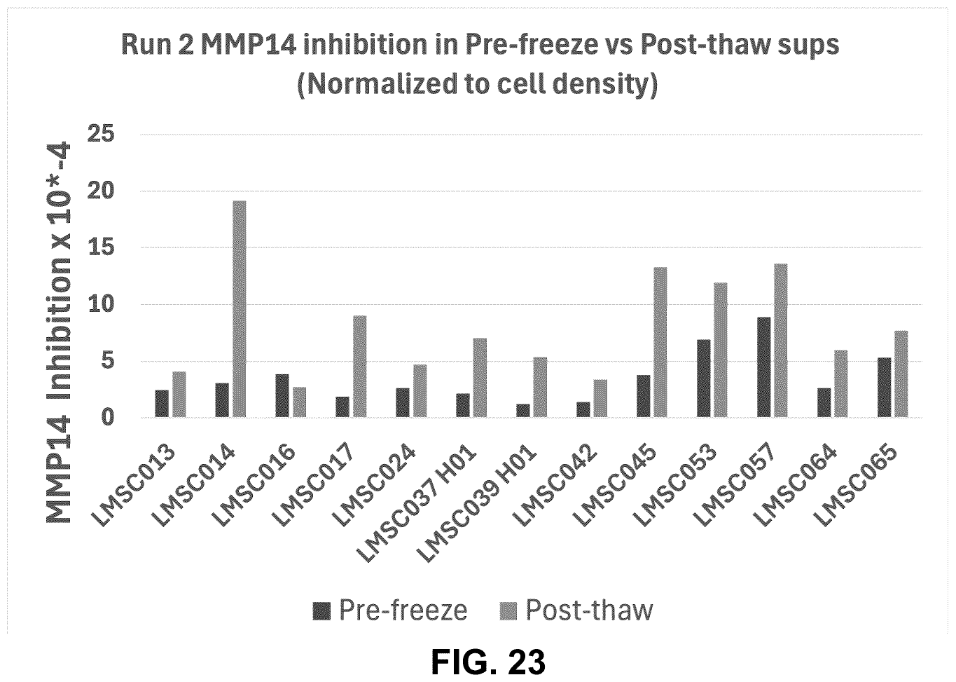

Referring now to FIG. 23, FIG. 23 depicts exemplary data comparing the MMP-14 inhibition potency between pre-freeze vs post-thaw Lomecel-B™ supernatants across 13 lots of Lomecel-B™ cells. MMP-14 inhibition level is normalized to cell density. Lomecel-B™ typically showed higher MMP14 inhibition in post-thaw samples (P4) compared to pre-freeze samples (P2).

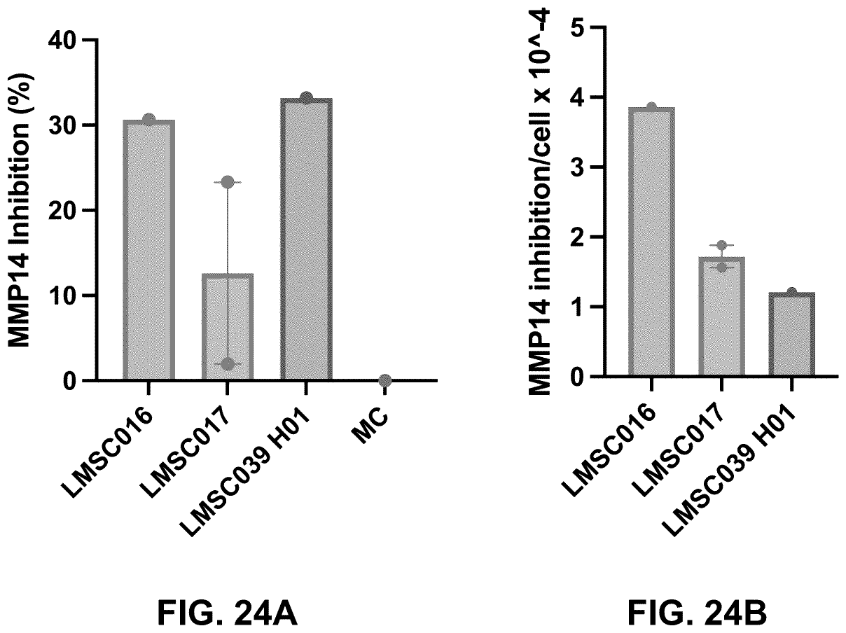

Referring now to FIGS. 24A-B, FIGS. 24A-B depict exemplary data showing significant inhibition of MMP-14 activity in all three tested Lomecel-B™ lots from the ELPIS Phase I clinical trial. It should be noted that substrate cleavage may not be specific to MMP-14 and could also be mediated by other MMPs in the base media, likely within the fetal bovine serum (FBS).

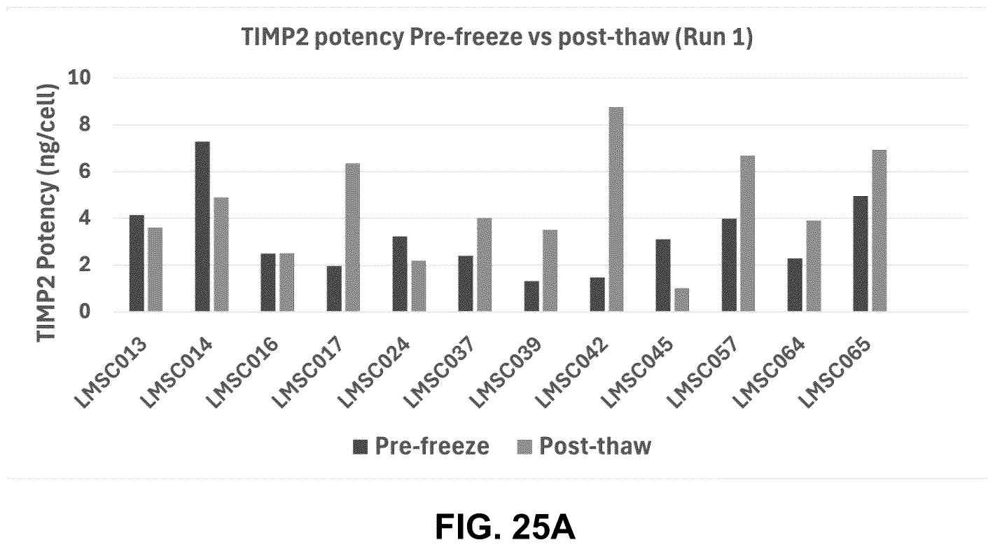

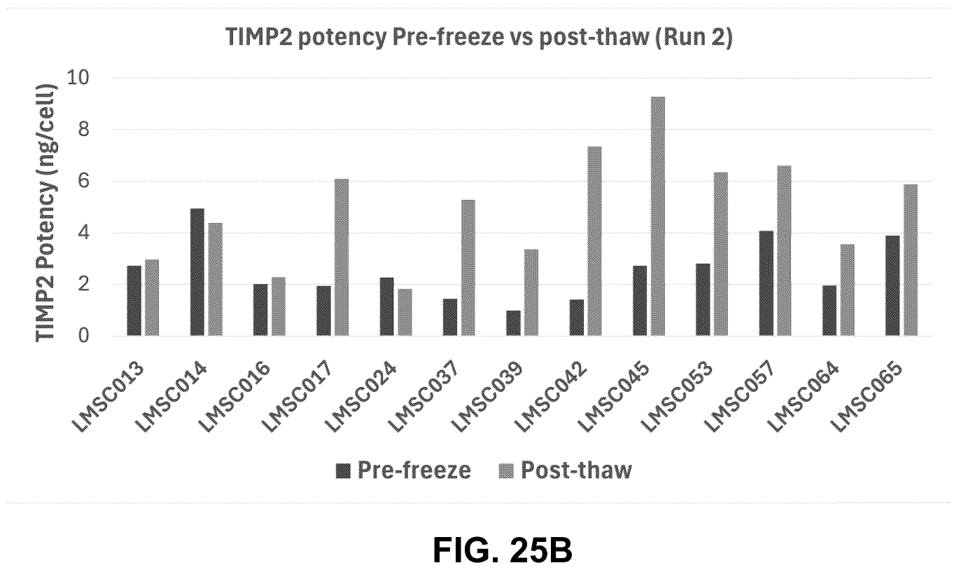

Referring now to FIGS. 25A-B, FIGS. 25A-B depict exemplary data based on two runs of TIMP2 potency assays (ELISA) using pre-freeze and post-thaw Lomecel-B™ supernatants. TIMP2 potency is not affected in the cell supernatants after a freeze thaw cycle (except for lots 014 and 042 in run 1). TIMP2 potency is elevated in most of the post-thaw lots in run 2.

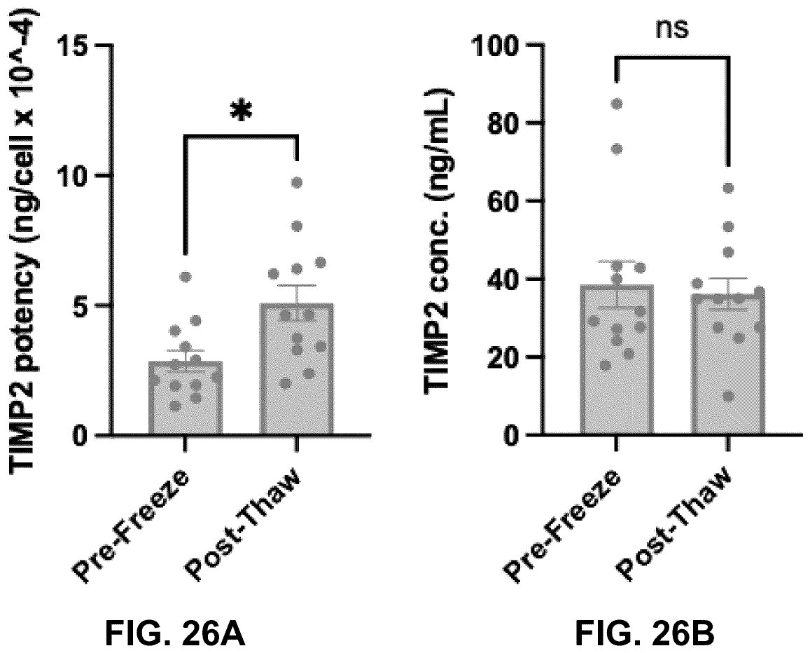

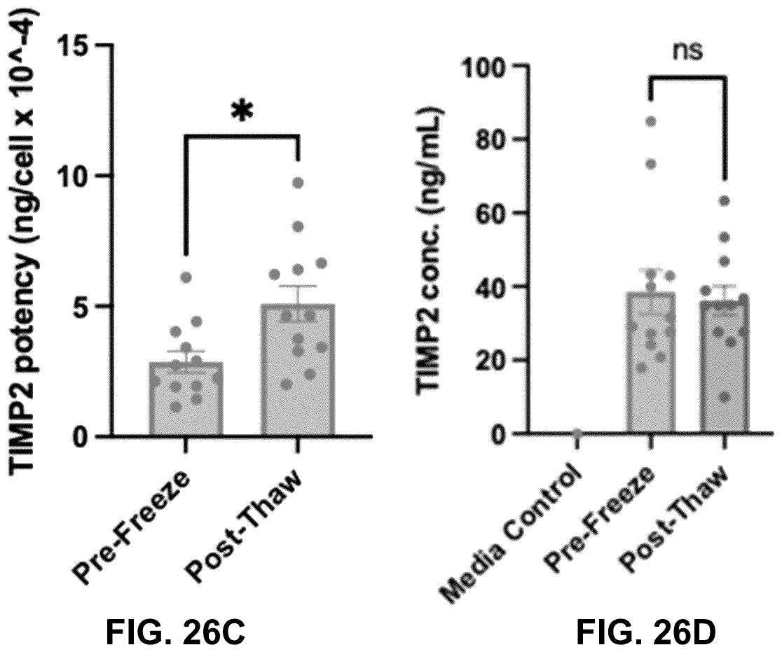

Referring now to FIGS. 26A-D, FIGS. 26A-D depict exemplary data comparing TIMP2 potency between pre-freeze and post-thaw Lomecel-B™ supernatants. As seen in FIGS. 26A and 26C, normalized TIMP2 potency is higher in post-thaw P3/P4 harvest supernatant compared to pre-freeze P2 supernatant. In contrast, as seen in FIGS. 26B and 26D, raw TIMP2 concentrations showed no significant difference.

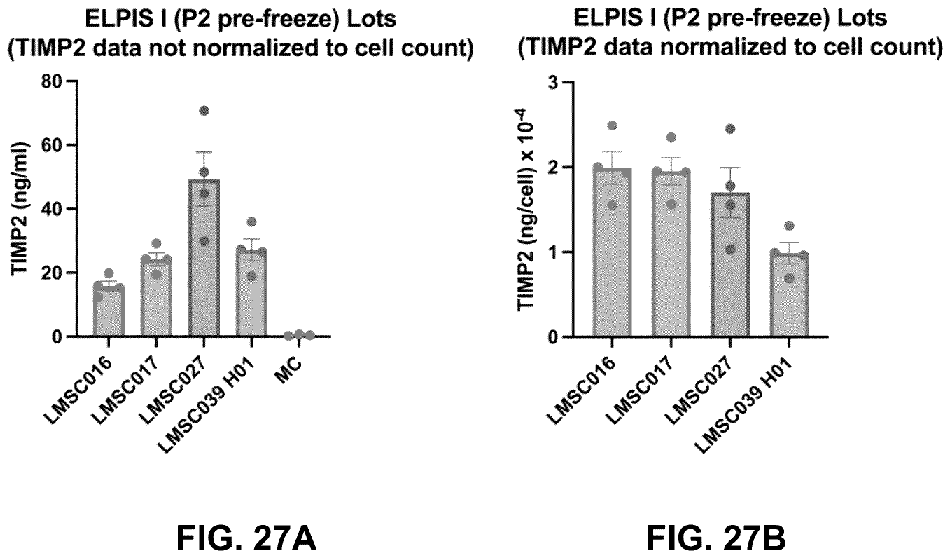

Referring now to FIGS. 27A-B, FIGS. 27A-B depict exemplary data comparing TIMP2 concentrations across four lots from the ELPIS Phase I clinical trial. FIG. 27A showed raw TIMP2 concentrations, whereas FIG. 27B showed normalized TIMP2 concentrations instead. All lots showed significant TIMP2 values.

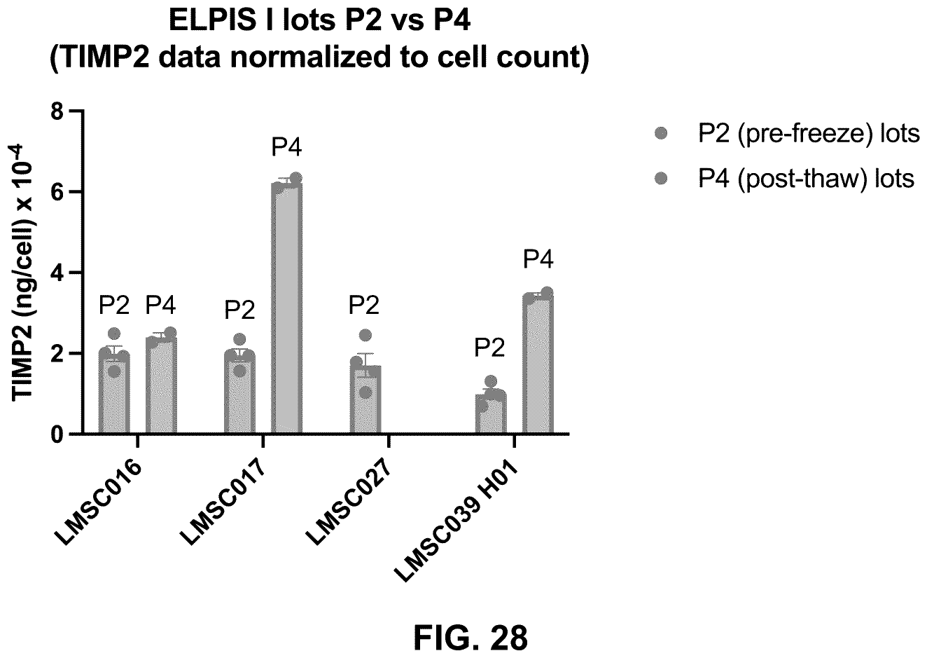

Referring now to FIG. 28, FIG. 28 depicts exemplary data showing normalized TIMP2 concentrations in both P2 (pre-freeze) and P4 (post-thaw) lots across four Lomecel-B™ lots. P4 values were generally higher than P2 values, consistent with the observations based on an aggregate of all clinical lots.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method for alleviating the symptoms of Alzheimer's disease (AD) in a subject in need thereof, the method comprising:
administering to the subject a composition comprising a therapeutically effective amount of allogeneic mesenchymal stem cells (MSCs); and
evaluating one or more biomarkers in the subject before and after administration of the composition, wherein the one or more biomarkers comprise one or more MRI biomarkers.

2. The method according to claim 1, further comprising measuring a cognitive or quality-of-life function of the subject before and after administration of the composition comprising the therapeutically effective amount of the allogenic MSCs.

3. The method according to claim 1, wherein the one or more MRI biomarkers are evaluated using a volume measurement of one or more members selected from a group consisting of a whole brain, one or more subregions of a whole brain, a lateral ventricle, grey matter, a medial temporal cortex, a hippocampus, a thalamus, white matter, and a cingulate cortex.

4. The method according to claim 3, wherein the volume measurement indicates a suppressed decrease in volume compared with placebo in one or more members selected from a group consisting of a whole brain, one or more subregions of a whole brain, grey matter, white matter, a medial temporal cortex, a hippocampus, and a thalamus of the subject after administration of the composition comprising the therapeutically effective amount of the allogenic MSCs.

5. The method according to claim 4, wherein the suppressed decrease is characterized by one or more members selected from a group consisting of at least 0.5% and no greater than 10%, at least 5% and no greater than 10%, at least 10% and no greater than 50%, and greater than 50%.

6. The method according to claim 3, wherein the volume measurement indicates a suppressed increase in volume compared to placebo in a brain ventricle or a lateral ventricle of the subject after administration of the composition comprising the therapeutically effective amount of the allogenic MSCs.

7. The method according to claim 6, wherein the suppressed increase is characterized by one or more members selected from a group consisting of at least 0.5% and no greater than 10%, at least 5% and no greater than 10%, at least 10% and no greater than 50%, and greater than 50%.

8. The method according to claim 1, wherein evaluating the one or more MRI biomarkers comprises evaluating the one or more MRI biomarkers using diffusion tensor imaging (DTI) or arterial spin labeling (ASL).

9. The method according to claim 1, wherein evaluating the one or more biomarkers comprises measuring a concentration of one or more members selected from a group consisting of one or more whole-blood biomarkers, one or more blood-plasma biomarkers, and one or more blood-serum biomarkers.

10. The method according to claim 1, wherein evaluating the one or more biomarkers comprises measuring a level of one or more members selected from a group consisting of matrix metalloproteinase-14 (MMP-14), tyrosine kinase with immunoglobulin and epidermal growth factor homology domains (TIE2) including soluble TIE2 (sTIE2), an angiopoietin-1 receptor, eotaxin 1, eotaxin 2, eotaxin 3, tissue-inhibitor-of-metalloprotease-2 (TIMP2), active glucose-dependent insulinotropic polypeptide (GIP), intact GIP, placental growth factor (plGF), amyloid beta peptide with 40 amino acids (Aβ40), interleukin 1β (IL-1β), interleukin 2 (IL-2), interleukin 4 (IL-4), interleukin 6 (IL-6), interleukin 10 (IL-10), and interleukin 13 (IL-13).

11. The method according to claim 1, wherein the composition comprises approximately 20×10⁶ MSCs.

12. The method according to claim 1, wherein the composition comprises approximately 100×10⁶ MSCs.

13. The method according to claim 1, wherein the composition comprises at least 20×10⁴ and no greater than 100×10⁸ MSCs.

14. The method according to claim 1, wherein the composition is configured to perform one or more therapeutic functions by preventing TIE2 from degradation.

15. The method according to claim 1, further comprising examining a cerebral spinal fluid of the subject before and after administration of the composition, wherein the composition further comprises allogeneic human microglial cells (HMCs).

16. A method for evaluating potency of allogeneic MSCs using a potency assay, wherein the potency assay includes an MMP-14 inhibition assay, a molecular assay for TIMP1, TIMP2, TIMP3, or VEGF-A, an enzyme-linked immunosorbent assay (ELISA), an electro-chemiluminescence assay, a meso scale discovery (MSD) assay, or an assay based on mass spectrometry.

17. The method according to claim 16, further comprising measuring an expression level of IL-6 or interleukin 8 (IL-8).

18. A method for evaluating potency of allogeneic MSCs using a potency assay, wherein the potency assay includes at least two members selected from a group consisting of an MMP-14 inhibition assay, a molecular assay for TIMP1, TIMP2, TIMP3, or VEGF-A, an enzyme-linked immunosorbent assay (ELISA), an electro-chemiluminescence assay, a meso scale discovery (MSD) assay, and an assay based on mass spectrometry.

19. The method according to claim 18, further comprising measuring an expression level of IL-6 or interleukin 8 (IL-8).

20. A method for treating, or alleviating symptoms of, hypoplastic left heart syndrome (HLHS) in a subject in need thereof, the method comprising:
administering to the subject a composition comprising a therapeutically effective amount of allogeneic mesenchymal stem cells (MSCs); and
evaluating one or more biomarkers in the subject before and after administration of the composition.

21. The method according to claim 20, wherein evaluating the one or more biomarkers comprises measuring a concentration of one or more members selected from a group consisting of one or more whole-blood biomarkers, one or more blood-plasma biomarkers, and one or more blood-serum biomarkers.

22. The method according to claim 20, wherein evaluating the one or more biomarkers comprises measuring a level of one or more members selected from a group consisting of matrix metalloproteinase-14 (MMP-14), tyrosine kinase with immunoglobulin and epidermal growth factor homology domains (TIE2) including soluble TIE2 (sTIE2), an angiopoietin-1 receptor, eotaxin 1, eotaxin 2, eotaxin 3, tissue-inhibitor-of-metalloprotease-2 (TIMP2), active glucose-dependent insulinotropic polypeptide (GIP), intact GIP, placental growth factor (plGF), amyloid beta peptide with 40 amino acids (Aβ40), interleukin 1β (IL-1β), interleukin 2 (IL-2), interleukin 4 (IL-4), interleukin 6 (IL-6), interleukin 10 (IL-10), and interleukin 13 (IL-13).

23. The method according to claim 20, wherein the composition comprises approximately 20×10⁶ MSCs.

24. The method according to claim 20, wherein the composition comprises approximately 100×10⁶ MSCs.

25. The method according to claim 20, wherein the composition comprises at least 20×10⁴ and no greater than 100×10⁸ MSCs.

26. The method according to claim 20, wherein the composition is configured to perform one or more therapeutic functions by preventing TIE2 from degradation.
